SEQUENCE LISTING (159 sequences)

SEQ ID NO: 1 - protein, 120 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 2 - protein, 114 aa, synthetic construct
  source 1..114: note = TIGIT-3 VH
EVQLVESGGGLVQPGGSLKLSCAASGFTFSSYGMSWVRQTPDKRLELVATIKSDGGSTNLPDSVKGRFTISRDNAKNTLHLQMSSLKSEDTAMYYCVRGYFDYWGQGTSVTVSS

SEQ ID NO: 3 - protein, 107 aa, synthetic construct
  source 1..107: note = TIGIT-3 VL
DIVMTQSHKFMSTSVGDRVSITCKASQDVKTAVAWYQQKPGQSPKLLIYWSSTRHTGVPDRFTGSRSLTDHTLTISSVQAEDLALYYCQQYYSTPYTFGGGTKLELK

SEQ ID NO: 4 - protein, 119 aa, synthetic construct
  source 1..119: note = TIGIT-6 VH
QVQLKQSGPELVKPGASVKISCKTSGYTITENTMHWVKQSHGKSLEWIGGINPNNGGTSYNQQFKGKATLTVDKSSSTAYMELRSLTSEDSAVYYCTRSSYYRYDLDYWGQGTTVTVSS

SEQ ID NO: 5 - protein, 107 aa, synthetic construct
  source 1..107: note = TIGIT-6 VL
DIQMTQSHKFMSTSVGDRVSITCKASQDVKTALAWYQQKPGQSPKLLIYSASYRYTGVPDRFTGSGSGTDFTFTISSVQAEDLAVYYCHQHYTTPWTFGGGTKLEIK

SEQ ID NO: 6 - protein, 120 aa, synthetic construct
  source 1..120: note = TIGIT-15 VH
DVQLVESGGGLVQPGGSLKLSCAASGFTFSSYGMSWVRQTPDRRLELVATINNNGGSTYYPDSVKGRFTISRDNARNTLYLQMSSLKSEDTAMYYCATRTGTEILGFDFWGQGTTLTVSS

SEQ ID NO: 7 - protein, 107 aa, synthetic construct
  source 1..107: note = TIGIT-15 VL
DIVLTQSHKFMSTSLGDRVSITCKASQDVSTAVAWYQQKPGQSPKLLIYWASSRHTGVPDRFTGSRSGPDYTLTISSVQAEDLALYYCQQQYSTPWTFGGGTKLEIK

SEQ ID NO: 8 - protein, 121 aa, synthetic construct
  source 1..121: note = TIGIT-18 VH
QVQLQQPGAELVRPGASVKLSCKASGYSFTSYWMNWVKQRPGQGHEWIGMIHLSNSETRLNQNFLDKATLTVDKSSSTVYLQLSSPTSEDSAVYYCARERDYYGSSYFDYWGQGTTLTVSS

SEQ ID NO: 9 - protein, 111 aa, synthetic construct
  source 1..111: note = TIGIT-18 VL
DIVMTQSPASLVVSLGQRATISCRTSQSVSTSSYSYMHWYQQKPGQPPKLLIKNASNLESGVPARFSASGSGTDFTLNIHPVEEEDAATYYCQHSWEIPRTFGGGTKLEIK

SEQ ID NO: 10 - protein, 119 aa, synthetic construct
  source 1..119: note = TIGIT-19 VH
QVQLQQPGPELVKPGTSVMLSCKASGYAFSRYSMYWVKQSHGKSLEWIAFIDPYNGGSTYNQRFRGKATLTVDKSSSTAYMHLNTLTSDDSAVYYCARGGDYGWYLGVWGAGTTVTVSS

SEQ ID NO: 11 - protein, 107 aa, synthetic construct
  source 1..107: note = TIGIT-19 VL
DIQMTQSPASLSASVGETVTITCRPSENIYTYLSWYQQKRGKSPQLLVYNAKTLPENVPSRFSGSGSGTQFSLKIDSLQPEDFGNYYCQHHYGNPLTFGAGTKLELK

SEQ ID NO: 12 - protein, 122 aa, synthetic construct
  source 1..122: note = TIGIT-52 VH
EVQLVESGGGLVEPGGSLTLSCVASGFTFSSFAMSWVRQTPEKRLEWVATISNGGSYTYYPDTVKGRFTISRDNAKNTLFLQMSSLRSEDTAMYYCARPYYGTTYGYYFDFWGQGTTLTVSS

SEQ ID NO: 13 - protein, 111 aa, synthetic construct
  source 1..111: note = TIGIT-52 VL
QIVLTQSPASLAVSLGQRATISCRASESVDRNGNSFMHWYQQKPGQPPKLLIYLASNLESGVPARFSGSGSRTDFTLTIDPVEADDAATYYCQQNNEDPLTFGAGTKLELK

SEQ ID NO: 14 - protein, 119 aa, synthetic construct
  source 1..119: note = TIGIT-63 VH
DVQLVESGGGLVQPGGSLSLSCAASGFTFTDYYMNWVRQPPGKALEWLGFIRNKANGYTTEYSASVKGRFTISRDNSQSILYLQMNALRAEDSATYYCARSTAWYFDVWGTGTTLTVSS

SEQ ID NO: 15 - protein, 107 aa, synthetic construct
  source 1..107: note = TIGIT-63 VL
DIQMTQSPASLSASVGGTVTITCRASEHIYSYLAWYQQKQGKSPQLLVYNAKTLAEGVPSRFSGSGSGTQFSLKINSLQPKDFGSYYCQHHYGNPLTFGAGTKLELK

SEQ ID NO: 16 - protein, 120 aa, synthetic construct
  source 1..120: note = TIGIT-87 VH
EVKLVESGPELVKPGASMKISCKASGYSFTGYTMNWVKQSHGKNLEWIGLINPYNGGTSYNQKFKGKATLTVDKSSSTAYMELLSLTSEDSAVYYCARYANRFENWFAYWGQGTLVTVSS

SEQ ID NO: 17 - protein, 107 aa, synthetic construct
  source 1..107: note = TIGIT-87 VL
DIQMTQSPASLSASVGETVTITCRASENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 18 - protein, 119 aa, synthetic construct
  source 1..119: note = TIGIT-94 VH
EVKLVESGGGLVKPGGSLKLSCAASGFTFSTYTMSWVRQTPEKRLEWVATISSGGTYTHYPDSVKGRFTISRDNAKNTLYLQMSSLKSEDTAMYYCTRWFLSLYAMDYWGQGTSVTVSS

SEQ ID NO: 19 - protein, 107 aa, synthetic construct
  source 1..107: note = TIGIT-94 VL
DIVMTQSTSSLSASLGDRVTISCRASQDINNFLNWYQQKPAGTVDLLIYYTSRLHSGVPSRFSGSGSGTDYSLTISNLDQEDIATYFCQQGNTFPYTFGGGTRLEIK

SEQ ID NO: 20 - protein, 119 aa, synthetic construct
  source 1..119: note = TIGIT-100 VH
QVQLQQPGPELVKPGASVKISCKASGYSFTDYNMNWVKQSNGKSLEWIGVINPNSGTDNYNQKFKGKATLTVDQSSSTAYMQLNSLTSEDSAVYFCARGVYFGNYFDSWGQGTTLTVSS

SEQ ID NO: 21 - protein, 107 aa, synthetic construct
  source 1..107: note = TIGIT-100 VL
DIVMTQSPASLSASVGETVTITCRASENIYSSLAWYQQKQGKSPQLLVFNAKTLTEGVPSRFSGSGSGTQFSLKINSLQPEDFGSYYCQHHYGAPWTFGGGTKLEIK

SEQ ID NO: 22 - protein, 114 aa, synthetic construct
  source 1..114: note = 3VH3
EVQLVESGGGLVQPGGSLRLSCAASGFTFSSYGMSWVRQAPGKGLELVATIKSDGGSTNLPDSVKGRFTISRDNSKNTLYLQMNSLRAEDTAVYYCVRGYFDYWGQGTTVTVSS

SEQ ID NO: 23 - protein, 114 aa, synthetic construct
  source 1..114: note = 3VH3.M4
EVQLVESGGGLVQPGGSLRLSCAASGFTFSSYGMSWVRQAPGKGLELVATIKSSGGSTNLADSVKGRFTISRDNSKNTLYLQMNSLRAEDTAVYYCVRGYFDYWGQGTTVTVSS

SEQ ID NO: 24 - protein, 107 aa, synthetic construct
  source 1..107: note = 3VL1
DIQMTQSPSSLSASVGDRVTITCKASQDVKTAVAWYQQKPGKVPKLLIYWSSTRHTGVPSRFSGSGSGTDFTLTISSLQPEDVATYYCQQYYSTPYTFGGGTKVEIK

SEQ ID NO: 25 - protein, 107 aa, synthetic construct
  source 1..107: note = 3VL1.M1
DIQMTQSPSSLSASVGDRVTITCRASQDIKTALAWYQQKPGKVPKLLIYWSSTRHTGVPSRFSGSGSGTDFTLTISSLQPEDVATYYCQQYYSTPYTFGGGTKVEIK

SEQ ID NO: 26 - protein, 107 aa, synthetic construct
  source 1..107: note = 3VL1.M3
DIQMTQSPSSLSASVGDRVTITCKASQDVKTAVAWYQQKPGKVPKLLIYWASTRHSGVPSRFSGSGSGTDFTLTISSLQPEDVATYYCQQYYSTPYTFGGGTKVEIK

SEQ ID NO: 27 - protein, 107 aa, synthetic construct
  source 1..107: note = 3VL1.M4
DIQMTQSPSSLSASVGDRVTITCKASQDVKTAVAWYQQKPGKVPKLLIYWASTRQSGVPSRFSGSGSGTDFTLTISSLQPEDVATYYCQQYYSTPYTFGGGTKVEIK

SEQ ID NO: 28 - protein, 107 aa, synthetic construct
  source 1..107: note = 3VL4.M6
DIQMTQSPSSLSASVGDRVTITCRASQDIKTALAWYQQKPGKSPKLLIYWASTRQSGVPSRFSGSGSGTDFTLTISSLQPEDVATYYCQQYYSTPYTFGGGTKVEIK

SEQ ID NO: 29 - protein, 107 aa, synthetic construct
  source 1..107: note = 3VL5.M6
DIQMTQSPSSLSASVGDRVTITCRASQDIKTALAWYQQKPGQSPKLLIYWASTRQSGVPSRFSGSGSGTDFTLTISSLQPEDVATYYCQQYYSTPYTFGGGTKVEIK

SEQ ID NO: 30 - protein, 119 aa, synthetic construct
  source 1..119: note = 6VH5
QVQLVQSGAEVKKPGASVKVSCKASGYTITENTMHWVRQAPGQRLEWIGGINPNNGGTSYNQQFKGRATLTRDTSASTAYMELSSLRSEDTAVYYCTRSSYYRYDLDYWGQGTTVTVSS

SEQ ID NO: 31 - protein, 119 aa, synthetic construct
  source 1..119: note = 6VH6
QVQLVQSGAEVKKPGASVKVSCKASGYTITENTMHWVRQAPGQRLEWIGGINPNNGGTSYNQQFKGRATLTVDTSASTAYMELSSLRSEDTAVYYCTRSSYYRYDLDYWGQGTTVTVSS

SEQ ID NO: 32 - protein, 107 aa, synthetic construct
  source 1..107: note = 6VL1
DIQMTQSPSSLSASVGDRVTITCKASQDVKTALAWYQQKPGKAPKLLIYSASYRYTGVPSRFSGSGSGTDFTFTISSLQPEDIATYYCHQHYTTPWTFGGGTKLEIK

SEQ ID NO: 33 - protein, 119 aa, synthetic construct
  source 1..119: note = 6VH6.M1
QVQLVQSGAEVKKPGASVKVSCKASGYTFTENAMHWVRQAPGQRLEWIGGINPNNGGTSYNQQFKGRATLTVDTSASTAYMELSSLRSEDTAVYYCTRSSYYRYDLDYWGQGTTVTVSS

SEQ ID NO: 34 - protein, 119 aa, synthetic construct
  source 1..119: note = 6VH6.M3
QVQLVQSGAEVKKPGASVKVSCKASGYTITENTMHWVRQAPGQRLEWIGGINPNNGGTSYSQKFQGRATLTVDTSASTAYMELSSLRSEDTAVYYCTRSSYYRYDLDYWGQGTTVTVSS

SEQ ID NO: 35 - protein, 107 aa, synthetic construct
  source 1..107: note = 6VL1.M1
DIQMTQSPSSLSASVGDRVTITCQASQDIKTALAWYQQKPGKAPKLLIYSASYRYTGVPSRFSGSGSGTDFTFTISSLQPEDIATYYCHQHYTTPWTFGGGTKLEIK

SEQ ID NO: 36 - protein, 119 aa, synthetic construct
  source 1..119: note = 6VH6.M4
QVQLVQSGAEVKKPGASVKVSCKASGYTITENTMHWVRQAPGQRLEWIGGINPNQGGTSYSQKFQGRATLTVDTSASTAYMELSSLRSEDTAVYYCTRSSYYRYDLDYWGQGTTVTVSS

SEQ ID NO: 37 - protein, 119 aa, synthetic construct
  source 1..119: note = 6VH6.M5
QVQLVQSGAEVKKPGASVKVSCKASGYTITENTMHWVRQAPGQRLEWIGGINPNNAGTSYSQKFQGRATLTVDTSASTAYMELSSLRSEDTAVYYCTRSSYYRYDLDYWGQGTTVTVSS

SEQ ID NO: 38 - protein, 119 aa, synthetic construct
  source 1..119: note = 6VH6.M6
QVQLVQSGAEVKKPGASVKVSCKASGYTFTENAMHWVRQAPGQRLEWIGGINPNQGGTSYSQKFQGRATLTVDTSASTAYMELSSLRSEDTAVYYCTRSSYYRYDLDYWGQGTTVTVSS

SEQ ID NO: 39 - protein, 119 aa, synthetic construct
  source 1..119: note = 6VH6.M7
QVQLVQSGAEVKKPGASVKVSCKASGYTFTENAMHWVRQAPGQRLEWIGGINPNNAGTSYSQKFQGRATLTVDTSASTAYMELSSLRSEDTAVYYCTRSSYYRYDLDYWGQGTTVTVSS

SEQ ID NO: 40 - protein, 119 aa, synthetic construct
  source 1..119: note = 19VH4
QVQLVQSGAEVKKPGASVKVSCKASGYAFSRYSMYWVRQAPGQGLEWIAFIDPYNGGSTYNQRFRGKATLTVDTSISTAYMELSRLRSDDTAVYYCARGGDYGWYLGVWGQGTLVTVSS

SEQ ID NO: 41 - protein, 107 aa, synthetic construct
  source 1..107: note = 19VL1
DIQMTQSPSSLSASVGDRVTITCRPSENIYTYLSWYQQKPGKAPKLLIYNAKTLPEGVPSRFSGSGSGTDFTLTISSLQPEDFATYYCQHHYGNPLTFGQGTKVEIK

SEQ ID NO: 42 - protein, 107 aa, synthetic construct
  source 1..107: note = 19VL1.M4
DIQMTQSPSSLSASVGDRVTITCRPSENIYTYLSWYQQKPGKAPKLLIYNAKSLPSGVPSRFSGSGSGTDFTLTISSLQPEDFATYYCQHHYGNPLTFGQGTKVEIK

SEQ ID NO: 43 - protein, 107 aa, synthetic construct
  source 1..107: note = 19VL1.M5
DIQMTQSPSSLSASVGDRVTITCRASQNIYTYLSWYQQKPGKAPKLLIYNAKSLPSGVPSRFSGSGSGTDFTLTISSLQPEDFATYYCQHHYGNPLTFGQGTKVEIK

SEQ ID NO: 44 - protein, 107 aa, synthetic construct
  source 1..107: note = 19VL1.M6
DIQMTQSPSSLSASVGDRVTITCRASQNIYSYLSWYQQKPGKAPKLLIYNAKSLPSGVPSRFSGSGSGTDFTLTISSLQPEDFATYYCQHHYGNPLTFGQGTKVEIK

SEQ ID NO: 45 - protein, 107 aa, synthetic construct
  source 1..107: note = 19VL1.M7
DIQMTQSPSSLSASVGDRVTITCRASQSIYSYLSWYQQKPGKAPKLLIYNAKSLPSGVPSRFSGSGSGTDFTLTISSLQPEDFATYYCQHHYGNPLTFGQGTKVEIK

SEQ ID NO: 46 - protein, 119 aa, synthetic construct
  source 1..119: note = 19VH4.M4
QVQLVQSGAEVKKPGASVKVSCKASGYAFSRYSMYWVRQAPGQGLEWIAFIDPYSGGSTYNQRFRGKATLTVDTSISTAYMELSRLRSDDTAVYYCARGGDYGWYLGVWGQGTLVTVSS

SEQ ID NO: 47 - protein, 119 aa, synthetic construct
  source 1..119: note = 19VH4.M5
QVQLVQSGAEVKKPGASVKVSCKASGYAFSRYSMYWVRQAPGQGLEWIAFIDPYNGGSTYAQKFQGKATLTVDTSISTAYMELSRLRSDDTAVYYCARGGDYGWYLGVWGQGTLVTVSS

SEQ ID NO: 48 - protein, 119 aa, synthetic construct
  source 1..119: note = 19VH4.M6
QVQLVQSGAEVKKPGASVKVSCKASGYTFTRYSMYWVRQAPGQGLEWIAFIDPYSGGSTYNQRFRGKATLTVDTSISTAYMELSRLRSDDTAVYYCARGGDYGWYLGVWGQGTLVTVSS

SEQ ID NO: 49 - protein, 119 aa, synthetic construct
  source 1..119: note = 19VH4.M7
QVQLVQSGAEVKKPGASVKVSCKASGYTFTRYSMYWVRQAPGQGLEWIAFIDPYSGGSTYAQKFQGKATLTVDTSISTAYMELSRLRSDDTAVYYCARGGDYGWYLGVWGQGTLVTVSS

SEQ ID NO: 50 - protein, 119 aa, synthetic construct
  source 1..119: note = 19VH4.M9
QVQLVQSGAEVKKPGASVKVSCKASGYTFTRYSMYWVRQAPGQGLEWIAFIDAYSGGSTYNQRFRGKATLTVDTSISTAYMELSRLRSDDTAVYYCARGGDYGWYLGVWGQGTLVTVSS

SEQ ID NO: 51 - protein, 119 aa, synthetic construct
  source 1..119: note = 19VH4.M10
QVQLVQSGAEVKKPGASVKVSCKASGYTFTRYSMYWVRQAPGQGLEWIAFIDAYSGGSTYAQKFQGKATLTVDTSISTAYMELSRLRSDDTAVYYCARGGDYGWYLGVWGQGTLVTVSS

SEQ ID NO: 52 - protein, 444 aa, synthetic construct
  source 1..444: note = TIGIT-3 chimeric heavy chain
EVQLVESGGGLVQPGGSLKLSCAASGFTFSSYGMSWVRQTPDKRLELVATIKSDGGSTNLPDSVKGRFTISRDNAKNTLHLQMSSLKSEDTAMYYCVRGYFDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 214 aa, synthetic construct
  source 1..214: note = TIGIT-3 chimeric light chain
DIVMTQSHKFMSTSVGDRVSITCKASQDVKTAVAWYQQKPGQSPKLLIYWSSTRHTGVPDRFTGSRSLTDHTLTISSVQAEDLALYYCQQYYSTPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 444 aa, synthetic construct
  source 1..444: note = 3VH3.M4 heavy chain
EVQLVESGGGLVQPGGSLRLSCAASGFTFSSYGMSWVRQAPGKGLELVATIKSSGGSTNLADSVKGRFTISRDNSKNTLYLQMNSLRAEDTAVYYCVRGYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 214 aa, synthetic construct
  source 1..214: note = 3VL4.M6 light chain
DIQMTQSPSSLSASVGDRVTITCRASQDIKTALAWYQQKPGKSPKLLIYWASTRQSGVPSRFSGSGSGTDFTLTISSLQPEDVATYYCQQYYSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 449 aa, synthetic construct
  source 1..449: note = TIGIT-6 chimeric heavy chain
QVQLKQSGPELVKPGASVKISCKTSGYTITENTMHWVKQSHGKSLEWIGGINPNNGGTSYNQQFKGKATLTVDKSSSTAYMELRSLTSEDSAVYYCTRSSYYRYDLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 214 aa, synthetic construct
  source 1..214: note = TIGIT-6 chimeric light chain
DIQMTQSHKFMSTSVGDRVSITCKASQDVKTALAWYQQKPGQSPKLLIYSASYRYTGVPDRFTGSGSGTDFTFTISSVQAEDLAVYYCHQHYTTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 449 aa, synthetic construct
  source 1..449: note = 6VH6.M4 heavy chain
QVQLVQSGAEVKKPGASVKVSCKASGYTITENTMHWVRQAPGQRLEWIGGINPNQGGTSYSQKFQGRATLTVDTSASTAYMELSSLRSEDTAVYYCTRSSYYRYDLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 449 aa, synthetic construct
  source 1..449: note = 6VH6.M5 heavy chain
QVQLVQSGAEVKKPGASVKVSCKASGYTITENTMHWVRQAPGQRLEWIGGINPNNAGTSYSQKFQGRATLTVDTSASTAYMELSSLRSEDTAVYYCTRSSYYRYDLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 449 aa, synthetic construct
  source 1..449: note = 6VH6.M6 heavy chain
QVQLVQSGAEVKKPGASVKVSCKASGYTFTENAMHWVRQAPGQRLEWIGGINPNQGGTSYSQKFQGRATLTVDTSASTAYMELSSLRSEDTAVYYCTRSSYYRYDLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 449 aa, synthetic construct
  source 1..449: note = 6VH6.M7 heavy chain
QVQLVQSGAEVKKPGASVKVSCKASGYTFTENAMHWVRQAPGQRLEWIGGINPNNAGTSYSQKFQGRATLTVDTSASTAYMELSSLRSEDTAVYYCTRSSYYRYDLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 214 aa, synthetic construct
  source 1..214: note = 6VL1.M1 light chain
DIQMTQSPSSLSASVGDRVTITCQASQDIKTALAWYQQKPGKAPKLLIYSASYRYTGVPSRFSGSGSGTDFTFTISSLQPEDIATYYCHQHYTTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 449 aa, synthetic construct
  source 1..449: note = TIGIT-19 chimeric heavy chain
QVQLQQPGPELVKPGTSVMLSCKASGYAFSRYSMYWVKQSHGKSLEWIAFIDPYNGGSTYNQRFRGKATLTVDKSSSTAYMHLNTLTSDDSAVYYCARGGDYGWYLGVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 214 aa, synthetic construct
  source 1..214: note = TIGIT-19 chimeric light chain
DIQMTQSPASLSASVGETVTITCRPSENIYTYLSWYQQKRGKSPQLLVYNAKTLPENVPSRFSGSGSGTQFSLKIDSLQPEDFGNYYCQHHYGNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 449 aa, synthetic construct
  source 1..449: note = 19VH4.M9 heavy chain
QVQLVQSGAEVKKPGASVKVSCKASGYTFTRYSMYWVRQAPGQGLEWIAFIDAYSGGSTYNQRFRGKATLTVDTSISTAYMELSRLRSDDTAVYYCARGGDYGWYLGVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 449 aa, synthetic construct
  source 1..449: note = 19VH4.M10 heavy chain
QVQLVQSGAEVKKPGASVKVSCKASGYTFTRYSMYWVRQAPGQGLEWIAFIDAYSGGSTYAQKFQGKATLTVDTSISTAYMELSRLRSDDTAVYYCARGGDYGWYLGVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 214 aa, synthetic construct
  source 1..214: note = 19VL1.M4 light chain
DIQMTQSPSSLSASVGDRVTITCRPSENIYTYLSWYQQKPGKAPKLLIYNAKSLPSGVPSRFSGSGSGTDFTLTISSLQPEDFATYYCQHHYGNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 214 aa, synthetic construct
  source 1..214: note = 19VL1.M7 light chain
DIQMTQSPSSLSASVGDRVTITCRASQSIYSYLSWYQQKPGKAPKLLIYNAKSLPSGVPSRFSGSGSGTDFTLTISSLQPEDFATYYCQHHYGNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 330 aa, synthetic construct
  source 1..330: note = human IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 107 aa, synthetic construct [sequence omitted]

SEQ ID NO: 71 - protein, 10 aa, synthetic construct
  source 1..10: note = TIGIT-3 HCDR1 (3VH/3VH3/3VH3.M4)
GFTFSSYGMS

SEQ ID NO: 72 - protein, 17 aa, synthetic construct
  source 1..17: note = TIGIT-3 HCDR2-1 (3VH/3VH3)
TIKSDGGSTNLPDSVKG

SEQ ID NO: 73 - protein, 17 aa, synthetic construct
  source 1..17: note = TIGIT-3 HCDR2-2 (3VH3.M4)
TIKSSGGSTNLADSVKG

SEQ ID NO: 74 - protein, 17 aa, synthetic construct
  source 1..17: note = TIGIT-3 HCDR2 Formula
  VARIANT 5: note = X is D or S
  VARIANT 12: note = X is P or A
TIKSXGGSTNLXDSVKG

SEQ ID NO: 75 - protein, 5 aa, synthetic construct
  source 1..5: note = TIGIT-3 HCDR3 (3VH/3VH3/3VH3.M4)
GYFDY

SEQ ID NO: 76 - protein, 11 aa, synthetic construct
  source 1..11: note = TIGIT-3 LCDR1-1 (3VL/3VL1/3VL1.M3/3VL1.M4)
KASQDVKTAVA

SEQ ID NO: 77 - protein, 11 aa, synthetic construct
  source 1..11: note = TIGIT-3 LCDR1-2 (3VL1.M1/3VL4.M6/3VL5.M6)
RASQDIKTALA

SEQ ID NO: 78 - protein, 11 aa, synthetic construct
  source 1..11: note = TIGIT-3 LCDR1 Formula
  VARIANT 1: note = X is K or R
  VARIANT 6: note = X is V or I
  VARIANT 10: note = X is V or L
XASQDXKTAXA

SEQ ID NO: 79 - protein, 7 aa, synthetic construct
  source 1..7: note = TIGIT-3 LCDR2-1 (3VL/3VL1/3VL1.M1)
WSSTRHT

SEQ ID NO: 80 - protein, 7 aa, synthetic construct
  source 1..7: note = TIGIT-3 LCDR2-2 (3VL1.M3)
WASTRHS

SEQ ID NO: 81 - protein, 7 aa, synthetic construct
  source 1..7: note = TIGIT-3 LCDR2-3 (3VL1.M4/3VL4.M6/3VL5.M6)
WASTRQS

SEQ ID NO: 82 - protein, 7 aa, synthetic construct
  source 1..7: note = TIGIT-3 LCDR2 Formula
  VARIANT 2: note = X is S or A
  VARIANT 6: note = X is H or Q
  VARIANT 7: note = X is T or S
WXSTRXX

SEQ ID NO: 83 - protein, 9 aa, synthetic construct
  source 1..9: note = TIGIT-3 LCDR3 (3VL/3VL1/3VL1.M1/3VL1.M3/3VL1.M4/3VL4.M6/3VL5.M6)
QQYYSTPYT

SEQ ID NO: 84 - protein, 10 aa, synthetic construct
  source 1..10: note = TIGIT-6 HCDR1-1 (6VH/6VH5/6VH6/6VH6.M3/6VH6.M4/6VH6.M5)
GYTITENTMH

SEQ ID NO: 85 - protein, 10 aa, synthetic construct
  source 1..10: note = TIGIT-6 HCDR1-2 (6VH6.M1/6VH6.M6/6VH6.M7)
GYTFTENAMH

SEQ ID NO: 86 - protein, 10 aa, synthetic construct
  source 1..10: note = TIGIT-6 HCDR1 Formula
  VARIANT 4: note = X is I or F
  VARIANT 8: note = X is T or A
GYTXTENXMH

SEQ ID NO: 87 - protein, 17 aa, synthetic construct
  source 1..17: note = TIGIT-6 HCDR2-1 (6VH/6VH5/6VH6/6VH6.M1)
GINPNNGGTSYNQQFKG

SEQ ID NO: 88 - protein, 17 aa, synthetic construct
  source 1..17: note = TIGIT-6 HCDR2-2 (6VH6.M3)
GINPNNGGTSYSQKFQG

SEQ ID NO: 89 - protein, 17 aa, synthetic construct
  source 1..17: note = TIGIT-6 HCDR2-3 (6VH6.M4/6VH6.M6)
GINPNQGGTSYSQKFQG

SEQ ID NO: 90 - protein, 17 aa, synthetic construct
  source 1..17: note = TIGIT-6 HCDR2-4 (6VH6.M5/6VH6.M7)
GINPNNAGTSYSQKFQG

SEQ ID NO: 91 - protein, 17 aa, synthetic construct
  source 1..17: note = TIGIT-6 HCDR2 Formula
  VARIANT 6: note = X is N or Q
  VARIANT 7: note = X is G or A
  VARIANT 12: note = X is N or S
  VARIANT 14: note = X is Q or K
  VARIANT 16: note = X is K or Q
GINPNXXGTSYXQXFXG

SEQ ID NO: 92 - protein, 10 aa, synthetic construct
  source 1..10: note = TIGIT-6 HCDR3 (6VH/6VH5/6VH6/6VH6.M1/6VH6.M3/6VH6.M 4/6VH6.M5/6VH6.M6/6VH6.M7)
SSYYRYDLDY

SEQ ID NO: 93 - protein, 11 aa, synthetic construct
  source 1..11: note = TIGIT-6 LCDR1-1 (6VL/6VL1)
KASQDVKTALA

SEQ ID NO: 94 - protein, 11 aa, synthetic construct
  source 1..11: note = TIGIT-6 LCDR1-2 (6VL1.M1)
QASQDIKTALA

SEQ ID NO: 95 - protein, 11 aa, synthetic construct
  source 1..11: note = TIGIT-6 LCDR1 Formula
  VARIANT 1: note = X is K or Q
  VARIANT 6: note = X is V or I
XASQDXKTALA

SEQ ID NO: 96 - protein, 7 aa, synthetic construct
  source 1..7: note = TIGIT-6 LCDR2 (6VL/6VL1/6VL1.M1)
SASYRYT

SEQ ID NO: 97 - protein, 9 aa, synthetic construct
  source 1..9: note = TIGIT-6 LCDR3 (6VL/6VL1/6VL1.M1)
HQHYTTPWT

SEQ ID NO: 98 - protein, 10 aa, synthetic construct
  source 1..10: note = TIGIT-19 HCDR1-1 (19VH/19VH4/19VH4.M4/19VH4.M5)
GYAFSRYSMY

SEQ ID NO: 99 - protein, 10 aa, synthetic construct
  source 1..10: note = TIGIT-19 HCDR1-2 (19VH4.M6/19VH4.M7/19VH4.M9/19VH4.M10)
GYTFTRYSMY

SEQ ID NO: 100 - protein, 10 aa, synthetic construct
  source 1..10: note = TIGIT-19 HCDR1 Formula
  VARIANT 3: note = X is A or T
  VARIANT 5: note = X is S or T
GYXFXRYSMY

SEQ ID NO: 101 - protein, 17 aa, synthetic construct
  source 1..17: note = TIGIT-19 HCDR2-1 (19VH/19VH4)
FIDPYNGGSTYNQRFRG

SEQ ID NO: 102 - protein, 17 aa, synthetic construct
  source 1..17: note = TIGIT-19 HCDR2-2 (19VH4.M4/19VH4.M6)
FIDPYSGGSTYNQRFRG

SEQ ID NO: 103 - protein, 17 aa, synthetic construct
  source 1..17: note = TIGIT-19 HCDR2-3 (19VH4.M5)
FIDPYNGGSTYAQKFQG

SEQ ID NO: 104 - protein, 17 aa, synthetic construct
  source 1..17: note = TIGIT-19 HCDR2-4 (19VH4.M7)
FIDPYSGGSTYAQKFQG

SEQ ID NO: 105 - protein, 17 aa, synthetic construct
  source 1..17: note = TIGIT-19 HCDR2-5 (19VH4.M9)
FIDAYSGGSTYNQRFRG

SEQ ID NO: 106 - protein, 17 aa, synthetic construct
  source 1..17: note = TIGIT-19 HCDR2-6 (19VH4.M10)
FIDAYSGGSTYAQKFQG

SEQ ID NO: 107 - protein, 17 aa, synthetic construct
  source 1..17: note = TIGIT-19 HCDR2 Formula
  VARIANT 4: note = X is P or A
  VARIANT 6: note = X is N or S
  VARIANT 12: note = X is N or A
  VARIANT 14: note = X is R or K
  VARIANT 16: note = X is R or Q
FIDXYXGGSTYXQXFXG

SEQ ID NO: 108 - protein, 10 aa, synthetic construct
  source 1..10: note = TIGIT-19 HCDR3 (19VH/19VH4/19VH4.M4/19VH4.M5/19VH4. M6/19VH4.M7/19VH4.M9/19VH4.M10)
GGDYGWYLGV

SEQ ID NO: 109 - protein, 11 aa, synthetic construct
  source 1..11: note = TIGIT-19 LCDR1-1 (19VL/19VL1/19VL1.M4)
RPSENIYTYLS

SEQ ID NO: 110 - protein, 11 aa, synthetic construct
  source 1..11: note = TIGIT-19 LCDR1-2 (19VL1.M5)
RASQNIYTYLS

SEQ ID NO: 111 - protein, 11 aa, synthetic construct
  source 1..11: note = TIGIT-19 LCDR1-3 (19VL1.M6)
RASQNIYSYLS

SEQ ID NO: 112 - protein, 11 aa, synthetic construct
  source 1..11: note = TIGIT-19 LCDR1-4 (19VL1.M7)
RASQSIYSYLS

SEQ ID NO: 113 - protein, 11 aa, synthetic construct
  source 1..11: note = TIGIT-19 LCDR1 Formula
  VARIANT 2: note = X is P or A
  VARIANT 4: note = X is E or Q
  VARIANT 5: note = X is N or S
  VARIANT 8: note = X is T or S
RXSXXIYXYLS

SEQ ID NO: 114 - protein, 7 aa, synthetic construct
  source 1..7: note = TIGIT-19 LCDR2-1 (19VL/19VL1)
NAKTLPE

SEQ ID NO: 115 - protein, 7 aa, synthetic construct
  source 1..7: note = TIGIT-19 LCDR2-2 (19VL1.M4/19VL1.M5/19VL1.M6/19VL1.M7)
NAKSLPS

SEQ ID NO: 116 - protein, 7 aa, synthetic construct
  source 1..7: note = TIGIT-19 LCDR2 Formula
  VARIANT 4: note = X is T or S
  VARIANT 7: note = X is E or S
NAKXLPX

SEQ ID NO: 117 - protein, 9 aa, synthetic construct
  source 1..9: note = TIGIT-19 LCDR3 (19VL/19VL1/19VL1.M4/19VL1.M5/19VL1.M6/19VL1.M7)
QHHYGNPLT

SEQ ID NO: 118 - protein, 10 aa, synthetic construct
  source 1..10: note = TIGIT-15 HCDR1
GFTFSSYGMS

SEQ ID NO: 119 - protein, 17 aa, synthetic construct
  source 1..17: note = TIGIT-15 HCDR2
TINNNGGSTYYPDSVKG

SEQ ID NO: 120 - protein, 11 aa, synthetic construct
  source 1..11: note = TIGIT-15 HCDR3
RTGTEILGFDF

SEQ ID NO: 121 - protein, 11 aa, synthetic construct
  source 1..11: note = TIGIT-15 LCDR1
KASQDVSTAVA

SEQ ID NO: 122 - protein, 7 aa, synthetic construct
  source 1..7: note = TIGIT-15 LCDR2
WASSRHT

SEQ ID NO: 123 - protein, 9 aa, synthetic construct
  source 1..9: note = TIGIT-15 LCDR3
QQQYSTPWT

SEQ ID NO: 124 - protein, 10 aa, synthetic construct
  source 1..10: note = TIGIT-18 HCDR1
GYSFTSYWMN

SEQ ID NO: 125 - protein, 17 aa, synthetic construct
  source 1..17: note = TIGIT-18 HCDR2
MIHLSNSETRLNQNFLD

SEQ ID NO: 126 - protein, 12 aa, synthetic construct
  source 1..12: note = TIGIT-18 HCDR3
ERDYYGSSYFDY

SEQ ID NO: 127 - protein, 15 aa, synthetic construct
  source 1..15: note = TIGIT-18 LCDR1
RTSQSVSTSSYSYMH

SEQ ID NO: 128 - protein, 7 aa, synthetic construct
  source 1..7: note = TIGIT-18 LCDR2
NASNLES

SEQ ID NO: 129 - protein, 9 aa, synthetic construct
  source 1..9: note = TIGIT-18 LCDR3
QHSWEIPRT

SEQ ID NO: 130 - protein, 10 aa, synthetic construct
  source 1..10: note = TIGIT-52 HCDR1
GFTFSSFAMS

SEQ ID NO: 131 - protein, 17 aa, synthetic construct
  source 1..17: note = TIGIT-52 HCDR2
TISNGGSYTYYPDTVKG

SEQ ID NO: 132 - protein, 13 aa, synthetic construct
  source 1..13: note = TIGIT-52 HCDR3
PYYGTTYGYYFDF

SEQ ID NO: 133 - protein, 15 aa, synthetic construct
  source 1..15: note = TIGIT-52 LCDR1
RASESVDRNGNSFMH

SEQ ID NO: 134 - protein, 7 aa, synthetic construct
  source 1..7: note = TIGIT-52 LCDR2
LASNLES

SEQ ID NO: 135 - protein, 9 aa, synthetic construct
  source 1..9: note = TIGIT-52 LCDR3
QQNNEDPLT

SEQ ID NO: 136 - protein, 10 aa, synthetic construct
  source 1..10: note = TIGIT-63 HCDR1
GFTFTDYYMN

SEQ ID NO: 137 - protein, 19 aa, synthetic construct
  source 1..19: note = TIGIT-63 HCDR2
FIRNKANGYTTEYSASVKG

SEQ ID NO: 138 - protein, 8 aa, synthetic construct
  source 1..8: note = TIGIT-63 HCDR3
STAWYFDV

SEQ ID NO: 139 - protein, 11 aa, synthetic construct
  source 1..11: note = TIGIT-63 LCDR1
RASEHIYSYLA

SEQ ID NO: 140 - protein, 7 aa, synthetic construct
  source 1..7: note = TIGIT-63 LCDR2
NAKTLAE

SEQ ID NO: 141 - protein, 9 aa, synthetic construct
  source 1..9: note = TIGIT-63 LCDR3
QHHYGNPLT

SEQ ID NO: 142 - protein, 10 aa, synthetic construct
  source 1..10: note = TIGIT-87 HCDR1
GYSFTGYTMN

SEQ ID NO: 143 - protein, 17 aa, synthetic construct
  source 1..17: note = TIGIT-87 HCDR2
LINPYNGGTSYNQKFKG

SEQ ID NO: 144 - protein, 11 aa, synthetic construct
  source 1..11: note = TIGIT-87 HCDR3
YANRFENWFAY

SEQ ID NO: 145 - protein, 11 aa, synthetic construct
  source 1..11: note = TIGIT-87 LCDR1
RASENIYSYLA

SEQ ID NO: 146 - protein, 7 aa, synthetic construct
  source 1..7: note = TIGIT-87 LCDR2
NAKTLAE

SEQ ID NO: 147 - protein, 9 aa, synthetic construct
  source 1..9: note = TIGIT-87 LCDR3
QHHYGTPWT

SEQ ID NO: 148 - protein, 10 aa, synthetic construct
  source 1..10: note = TIGIT-94 HCDR1
GFTFSTYTMS

SEQ ID NO: 149 - protein, 17 aa, synthetic construct
  source 1..17: note = TIGIT-94 HCDR2
TISSGGTYTHYPDSVKG

SEQ ID NO: 150 - protein, 10 aa, synthetic construct
  source 1..10: note = TIGIT-94 HCDR3
WFLSLYAMDY

SEQ ID NO: 151 - protein, 11 aa, synthetic construct
  source 1..11: note = TIGIT-94 LCDR1
RASQDINNFLN

SEQ ID NO: 152 - protein, 7 aa, synthetic construct
  source 1..7: note = TIGIT-94 LCDR2
YTSRLHS

SEQ ID NO: 153 - protein, 9 aa, synthetic construct
  source 1..9: note = TIGIT-94 LCDR3
QQGNTFPYT

SEQ ID NO: 154 - protein, 10 aa, synthetic construct
  source 1..10: note = TIGIT-100 HCDR1
GYSFTDYNMN

SEQ ID NO: 155 - protein, 17 aa, synthetic construct
  source 1..17: note = TIGIT-100 HCDR2
VINPNSGTDNYNQKFKG

SEQ ID NO: 156 - protein, 10 aa, synthetic construct
  source 1..10: note = TIGIT-100 HCDR3
GVYFGNYFDS

SEQ ID NO: 157 - protein, 11 aa, synthetic construct
  source 1..11: note = TIGIT-100 LCDR1
RASENIYSSLA

SEQ ID NO: 158 - protein, 7 aa, synthetic construct
  source 1..7: note = TIGIT-100 LCDR2
NAKTLTE

SEQ ID NO: 159 - protein, 9 aa, synthetic construct
  source 1..9: note = TIGIT-100 LCDR3
QHHYGAPWT

(19) United States
(12) Patent Application Publication
(10) Pub. No.: US 2026/0139048 A1
(43) Pub. Date: May 21, 2026

(54) ANTIBODIES TARGETING TIGIT AND USES THEREOF

(71) Applicant: Nanjing Legend Biotech Co., Ltd., Nanjing (CN)

(72) Inventors: Shuai YANG, Nanjing (CN); Sujuan WANG, Nanjing (CN); Jian LIU, Nanjing (CN); Yafeng ZHANG, Nanjing (CN); Qing SUN, Nanjing (CN); Wei SUN, Nanjing (CN); Shu WU, Nanjing (CN); An TANG, Nanjing (CN)

(21) Appl. No.: 19/102,746

(22) PCT Filed: Aug. 25, 2023
(86) PCT No.: PCT/CN2023/114953

§ 371 Date: Feb. 10, 2025

(30) Foreign Application Priority Data
Aug. 26, 2022 (WO) PCT/CN2022/115189

(51) Int. Cl. C07K 16/28; A61K 39/00; A61K 45/06; A61P 35/00

(52) U.S. Cl. CPC C07K 16/28; A61K 45/06; A61P 35/00; A61K 2039/505; C07K 2317/24; C07K 2317/732; C07K 2317/76; C07K 2317/92

(57) ABSTRACT

The present disclosure relates to an antibody, such as a monoclonal antibody (mAb), or an antigen-binding fragment thereof, that specifically recognizes TIGIT, and methods of making the same and using the same.

### CROSS REFERENCE TO RELATED APPLICATION

This application is a 371 national stage application of PCT Application No. PCT/CN2023/114953, filed on Aug. 25, 2023, which claims priority to PCT Application No. PCT/CN2022/115189, filed on Aug. 26, 2022. The disclosures of the aforementioned applications are hereby incorporated by reference in their entireties.

### SEQUENCE STATEMENT

This application contains a Sequence Listing that has been submitted electronically as an XML file named “51624-0110US1_SL_ST26.XML.” The XML file, created on Dec. 16, 2025, is 190,571 bytes in size. The material in the XML file is hereby incorporated by reference in its entirety.

### TECHNICAL FIELD

The present disclosure relates to an antibody, such as a monoclonal antibody (mAb), or an antigen-binding fragment thereof, that specifically recognizes TIGIT, and methods of making the same and using the same.

### BACKGROUND ART

T cell immunoreceptor with Ig and ITIM domains (TIGIT, also known as Vstm3 or WUCAM) is an immune receptor belonging to the CD28 family. This 26 KDa protein contains an extracellular IgV domain, a type I transmembrane region, an intracellular immunoglobulin tail tyrosine (ITT)-like motif, and a C-terminal immunereceptor tyrosine-based inhibition motif (ITIM) in cytoplasm. In naive T cells and NK cells, TIGIT is barely detectable on the cell surface but is upregulated upon T cell and NK cell activation. In tumor microenvironment, TIGIT is highly detected on regulatory T cells (Treg), exhausted T cells and NK cells. TIGIT has multiple ligands, including CD155 (necl-5 or poliovirus receptor (PVR)), CD112 (Nectin-2 or Poliovirus receptor-related 2 (PVRL2)), and CD113 (Nectin-3 or PVRL3). TIGIT can bind to CD155 (PVR) with high affinity, while to CD112 and CD113 with lower affinity. Recent reports also indicate that TIGIT interacts with CD226 (PTA1 or DNAM-1) in cis.

TIGIT exerts its inhibitory immune checkpoint function via several mechanisms. First, upon binding to its major ligand CD155 (PVR), the subsequent phosphorylation of TIGIT in its ITIM domain transduces inhibitory signals to downregulate IFN-γ expression in T cells and NK cells via NF-κB pathway. Second, as TIGIT interacts with PVR at higher affinity than with CD226, it competes with CD226 and attenuates the stimulatory signal transduced by CD226. Third, PVR binding to TIGIT on dendritic cells may lead to upregulation of IL-10 expression and downregulation of IL-12 expression, therefore impairing the anti-tumor immune response of dendritic cells. Lastly, recent research indicated that TIGIT can directly bind to CD226 in cis to inhibit CD226 dimerization, which is required for T cell activation. Therefore, TIGIT acts as an important negative regulator in immune responses in infection and cancer, and blockade of TIGIT signaling has been proposed as an approach to enhance T cell and NK cell immunity for cancer treatment.

### SUMMARY OF DISCLOSURE

The present disclosure provides antibodies that specifically bind to TIGIT (e.g., human TIGIT) and block binding to PVR/CD155, thereby reducing or eliminating TIGIT-mediated immune suppression. Also provided are pharmaceutical compositions comprising these antibodies, nucleic acids encoding these antibodies, vectors and host cells for making these antibodies, and methods of treating a subject using these antibodies. The antibodies disclosed herein have showed better tumor inhibition than two anti-TIGIT reference antibodies that are in advanced clinical stages even at suboptimal doses, holding great promise for the treatment of diseases that involve immune suppression.

In one aspect, the disclosure provides an antibody or antigen-binding fragment thereof, that binds to TIGIT, comprising:
(i) HCDR1 comprising the sequence of SEQ ID NO: 84-86, 98-100, 71, 118, 124, 130, 136, 142, 148, or 154, e.g., the sequence of SEQ ID NO: 84-85, 98-99, 71, 118, 124, 130, 136, 142, 148, or 154;
(ii) HCDR2 comprising the sequence of SEQ ID NO: 87-91, 101-107, 72-74, 119, 125, 131, 137, 143, 149, or 155, e.g., the sequence of SEQ ID NO: 87-90, 101-106, 72-73, 119, 125, 131, 137, 143, 149, or 155;
(iii) HCDR3 comprising the sequence of SEQ ID NO: 92, 108, 75, 120, 126, 132, 138, 144, 150, or 156;
(iv) LCDR1 comprising the sequence of SEQ ID NO: 93-95, 109-113, 76-78, 121, 127, 133, 139, 145, 151, or 157, e.g., the sequence of SEQ ID NO: 93-94, 109-112, 76-77, 121, 127, 133, 139, 145, 151, or 157;
(v) LCDR2 comprising the sequence of SEQ ID NO: 96, 114-116, 79-82, 122, 128, 134, 140, 146, 152, or 158, e.g., the sequence of SEQ ID NO: 96, 114-115, 79-81, 122, 128, 134, 140, 146, 152, or 158; and
(vi) LCDR3 comprising the sequence of SEQ ID NO: 97, 117, 83, 123, 129, 135, 141, 147, 153, or 159.

### TIGIT-6

In certain embodiments, the antibody or antigen-binding fragment thereof of the disclosure comprises:
(i) HCDR1 comprising the sequence of GYTX₁TENX₂MH (SEQ ID NO: 86), wherein X₁ is I or F, X₂ is T or A;
(ii) HCDR2 comprising the sequence of GINPNX₃X₄GTSYX₅QX₆FX₇G (SEQ ID NO: 91), wherein X₃ is N or Q, X₄ is G or A, X₅ is N or S, X₆ is Q or K, X₇ is K or Q;
(iii) HCDR3 comprising the sequence of SEQ ID NO: 92;
(iv) LCDR1 comprising the sequence of X₈ASQDX₉KTALA (SEQ ID NO: 95), wherein X₈ is K or Q, X₉ is V or I;
(v) LCDR2 comprising the sequence of SEQ ID NO: 96; and
(vi) LCDR3 comprising the sequence of SEQ ID NO: 97.

In certain embodiments, the HCDR1 comprises the sequence of SEQ ID NO: 84 or 85. In certain embodiments, the HCDR2 comprises the sequence of SEQ ID NO: 87, 88, 89, or 90. In certain embodiments, the LCDR1 comprises the sequence of SEQ ID NO: 93 or 94.

In certain embodiments, the antibody or antigen-binding fragment thereof comprises:
(1) HCDR1, HCDR2 and HCDR3 comprising the amino acid sequences of SEQ ID NOs: 84, 87, and 92, respectively; and LCDR1, LCDR2 and LCDR3 comprising the amino acid sequences of SEQ ID NOs: 93, 96, and 97, respectively; or,
(2) HCDR1, HCDR2 and HCDR3 comprising the amino acid sequences of: (a) SEQ ID NOs: 85, 87, and 92, respectively; or (b) SEQ ID NOs: 84, 88, and 92, respectively; or (c) SEQ ID NOs: 84, 89, and 92, respectively; and LCDR1, LCDR2 and LCDR3 comprising the amino acid sequences of SEQ ID NOs: 93, 96, and 97, respectively; or,
(3) HCDR1, HCDR2 and HCDR3 comprising the amino acid sequences of: (a) SEQ ID NOs: 84, 87, and 92, respectively; or (b) SEQ ID NOs: 84, 88, and 92, respectively; or (c) SEQ ID NOs: 84, 89, and 92, respectively; or (d) SEQ ID NOs: 84, 90, and 92, respectively; or (e) SEQ ID NOs: 85, 89, and 92, respectively; or (f) SEQ ID NOs: 85, 90, and 92, respectively; and LCDR1, LCDR2 and LCDR3 comprising the amino acid sequences of SEQ ID NOs: 94, 96, and 97, respectively.

In certain embodiments, the antibody or antigen-binding fragment thereof comprises a heavy chain variable region (VH) and a light chain variable region (VL), wherein: the VH comprises the sequence of SEQ ID NO: 4 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 5 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto. In certain embodiments, the VH comprises the sequence of SEQ ID NO: 4; and/or, the VL comprises the sequence of SEQ ID NO: 5.

In certain embodiments, the antibody or antigen-binding fragment thereof comprises a heavy chain variable region (VH) and a light chain variable region (VL), wherein: the VH comprises framework regions (FRs) derived from a human immunoglobulin heavy chain variable region sequence of GenBank: QVG74320; and/or, the VL comprises framework regions (FRs) derived from a human immunoglobulin light chain variable region sequence of GenBank: ACY78416.

In certain embodiments, the VH comprises the sequence of SEQ ID NO: 30, 31, 33, 34, 36, 37, 38, or 39, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VL comprises the sequence of SEQ ID NO: 32 or 35, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises a VH and a VL, wherein:
(1) the VH comprises the sequence of SEQ ID NO: 30, 31, 33, 34, or 36, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 32 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
(2) the VH comprises the sequence of SEQ ID NO: 31, 34, 36, 37, 38, or 39, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 35 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VH comprises the sequence of SEQ ID NO: 36, 37, 38, or 39, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 35 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto. In certain embodiments, the VH comprises the sequence of SEQ ID NO: 36, 37, 38, or 39; and/or, the VL comprises the sequence of SEQ ID NO: 35.

In certain embodiments, (a) the VH comprises the sequence of SEQ ID NO: 36; and/or, the VL comprises the sequence of SEQ ID NO: 35; (b) the VH comprises the sequence of SEQ ID NO: 37; and/or, the VL comprises the sequence of SEQ ID NO: 35; (c) the VH comprises the sequence of SEQ ID NO: 38; and/or, the VL comprises the sequence of SEQ ID NO: 35; or, (d) the VH comprises the sequence of SEQ ID NO: 39; and/or, the VL comprises the sequence of SEQ ID NO: 35.

### TIGIT-19

In certain embodiments, the antibody or antigen-binding fragment thereof of the disclosure comprises:
(i) HCDR1 comprising the sequence of GYX₁FX₂RYSMY (SEQ ID NO: 100), wherein X₁ is A or T, X₂ is S or T;
(ii) HCDR2 comprising the sequence of FIDX₃YX₄GGSTYX₅QX₆FX₇G (SEQ ID NO: 107), wherein X₃ is P or A, X₄ is N or S, X₅ is N or A, X₆ is R or K, X₇ is R or Q;
(iii) HCDR3 comprising the sequence of SEQ ID NO: 108;
(iv) LCDR1 comprising the sequence of RX₈SX₉X₁₀IYX₁₁YLS (SEQ ID NO: 113), wherein X₈ is P or A, X₉ is E or Q, X₁₀ is N or S, X₁₁ is T or S;
(v) LCDR2 comprising the sequence of NAKX₁₂LPX₁₃ (SEQ ID NO: 116), wherein X₁₂ is T or S, X₁₃ is E or S; and
(vi) LCDR3 comprising the sequence of SEQ ID NO: 117.

In certain embodiments, the HCDR1 comprises the sequence of SEQ ID NO: 98 or 99. In certain embodiments, the HCDR2 comprises the sequence of SEQ ID NO: 101, 102, 103, 104, 105, or 106. In certain embodiments, the LCDR1 comprises the sequence of SEQ ID NO: 109, 110, 111, or 112. In certain embodiments, the LCDR2 comprises the sequence of SEQ ID NO: 114 or 115.

In certain embodiments, the antibody or antigen-binding fragment thereof comprises:
(1) HCDR1, HCDR2 and HCDR3 comprising the amino acid sequences of SEQ ID NOs: 98, 101, and 108, respectively; and LCDR1, LCDR2 and LCDR3 comprising the amino acid sequences of SEQ ID NOs: 109, 114, and 117, respectively; or,
(2) HCDR1, HCDR2 and HCDR3 comprising the amino acid sequences of SEQ ID NOs: 98, 101 and 108, respectively; and LCDR1, LCDR2 and LCDR3 comprising the amino acid sequences of: (a) SEQ ID NOs: 109, 115 and 117, respectively; or (b) SEQ ID NOs: 110, 115 and 117, respectively; or (c) SEQ ID NOs: 111, 115 and 117, respectively; or (d) SEQ ID NOs: 112, 115 and 117, respectively; or,
(3) HCDR1, HCDR2 and HCDR3 comprising the amino acid sequences of: (a) SEQ ID NOs: 98, 102, and 108, respectively; or (b) SEQ ID NOs: 98, 103, and 108, respectively; or (c) SEQ ID NOs: 99, 102, and 108, respectively; or (d) SEQ ID NOs: 99, 104, and 108, respectively; or (e) SEQ ID NOs: 99, 105, and 108, respectively; and LCDR1, LCDR2 and LCDR3 comprising the amino acid sequences of SEQ ID NOs: 109, 114 and 117, respectively; or,
(4) HCDR1, HCDR2 and HCDR3 comprising the amino acid sequences of SEQ ID NOs: 99, 105 and 108, respectively; and LCDR1, LCDR2 and LCDR3 comprising the amino acid sequences of: (a) SEQ ID NOs: 109, 115 and 117, respectively; or (b) SEQ ID NOs: 112, 115 and 117, respectively; or,
(5) HCDR1, HCDR2 and HCDR3 comprising the amino acid sequences of SEQ ID NOs: 99, 106 and 108, respectively; and LCDR1, LCDR2 and LCDR3 comprising the amino acid sequences of: (a) SEQ ID NOs: 109, 115 and 117, respectively; or (b) SEQ ID NOs: 112, 115 and 117, respectively.

In certain embodiments, the antibody or antigen-binding fragment thereof comprises a heavy chain variable region (VH) and a light chain variable region (VL), wherein: the VH comprises the sequence of SEQ ID NO: 10 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 11 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto. In certain embodiments, the VH comprises the sequence of SEQ ID NO: 10; and/or, the VL comprises the sequence of SEQ ID NO: 11.

In certain embodiments, the antibody or antigen-binding fragment thereof comprises a heavy chain variable region (VH) and a light chain variable region (VL), wherein: the VH comprises framework regions (FRs) derived from a human immunoglobulin heavy chain variable region sequence of GenBank: AXA20212; and/or, the VL comprises framework regions (FRs) derived from a human immunoglobulin light chain variable region sequence of GenBank: ABA70776.

In certain embodiments, the VH comprises the sequence of SEQ ID NO: 40, 46, 47, 48, 49, 50, or 51, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VL comprises the sequence of SEQ ID NO: 41, 42, 43, 44, or 45, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises a VH and a VL, wherein:
(1) the VH comprises the sequence of SEQ ID NO: 40, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 41, 42, 43, 44, or 45, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
(2) the VH comprises the sequence of SEQ ID NO: 46, 47, 48, 49, or 50, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 41 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
(3) the VH comprises the sequence of SEQ ID NO: 50, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 42 or 45, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
(4) the VH comprises the sequence of SEQ ID NO: 51, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 42 or 45, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VH comprises the sequence of SEQ ID NO: 50 or 51, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 42 or 45, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto. In certain embodiments, the VH comprises the sequence of SEQ ID NO: 50 or 51; and/or, the VL comprises the sequence of SEQ ID NO: 42 or 45.

In certain embodiments, (a) the VH comprises the sequence of SEQ ID NO: 50; and/or, the VL comprises the sequence of SEQ ID NO: 42; (b) the VH comprises the sequence of SEQ ID NO: 50; and/or, the VL comprises the sequence of SEQ ID NO: 45; (c) the VH comprises the sequence of SEQ ID NO: 51; and/or, the VL comprises the sequence of SEQ ID NO: 42; or (d) the VH comprises the sequence of SEQ ID NO: 51; and/or, the VL comprises the sequence of SEQ ID NO: 45.

### TIGIT-3

In certain embodiments, the antibody or antigen-binding fragment thereof of the disclosure comprises:
(i) HCDR1 comprising the sequence of SEQ ID NO: 71;
(ii) HCDR2 comprising the sequence of TIKSX₁GGSTNLX₂DSVKG (SEQ ID NO: 74), wherein X₁ is D or S, X₂ is P or A;
(iii) HCDR3 comprising the sequence of SEQ ID NO: 75;
(iv) LCDR1 comprising the sequence of X₃ASQDX₄KTAX₅A (SEQ ID NO: 78), wherein X₃ is Kor R, X₄ is V or I, X₅ is V or L;
(v) LCDR2 comprising the sequence of WX₆STRX₇X₈ (SEQ ID NO: 82), wherein X₆ is S or A, X₇ is H or Q, X₈ is T or S; and
(vi) LCDR3 comprising the sequence of SEQ ID NO: 83.

In certain embodiments, the HCDR2 comprises the sequence of SEQ ID NO: 72 or 73. In certain embodiments, the LCDR1 comprises the sequence of SEQ ID NO: 76 or 77. In certain embodiments, the LCDR2 comprises the sequence of SEQ ID NO: 79, 80, or 81.

In certain embodiments, the antibody or antigen-binding fragment thereof comprises:
(1) HCDR1, HCDR2 and HCDR3 comprising the amino acid sequences of SEQ ID NOs: 71, 72, and 75, respectively; and LCDR1, LCDR2 and LCDR3 comprising the amino acid sequences of SEQ ID NOs: 76, 79, and 83, respectively; or,
(2) HCDR1, HCDR2 and HCDR3 comprising the amino acid sequences of SEQ ID NOs: 71, 73, and 75, respectively; and LCDR1, LCDR2 and LCDR3 comprising the amino acid sequences of SEQ ID NOs: 76, 79, and 83, respectively; or,
(3) HCDR1, HCDR2 and HCDR3 comprising the amino acid sequences of SEQ ID NOs: 71, 72, and 75; and LCDR1, LCDR2 and LCDR3 comprising the amino acid sequences of: (a) SEQ ID NOs: 76, 79, and 83, respectively; or (b) SEQ ID NOs: 77, 79, and 83, respectively; or (c) SEQ ID NOs: 76, 80, and 83, respectively; or (d) SEQ ID NOs: 76, 81, and 83, respectively; or,
(4) HCDR1, HCDR2 and HCDR3 comprising the amino acid sequences of SEQ ID NOs: 71, 73, and 75, respectively; and LCDR1, LCDR2 and LCDR3 comprising the amino acid sequences of SEQ ID NOs: 77, 81, and 83, respectively.

In certain embodiments, the antibody or antigen-binding fragment thereof comprises a heavy chain variable region (VH) and a light chain variable region (VL), wherein: the VH comprises the sequence of SEQ ID NO: 2 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 3 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto. In certain embodiments, the VH comprises the sequence of SEQ ID NO: 2; and/or, the VL comprises the sequence of SEQ ID NO: 3.

In certain embodiments, the antibody or antigen-binding fragment thereof comprises a heavy chain variable region (VH) and a light chain variable region (VL), wherein: the VH comprises framework regions (FRs) derived from a human immunoglobulin heavy chain variable region sequence of GenBank: BAC02278; and/or, the VL comprises framework regions (FRs) derived from a human immunoglobulin light chain variable region sequence of GenBank: QTX15665.

In certain embodiments, the VH comprises the sequence of SEQ ID NO: 22 or 23, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VL comprises the sequence of SEQ ID NO: 24, 25, 26, 27, 28, or 29, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises a VH and a VL, wherein:
(1) the VH comprises the sequence of SEQ ID NO: 22 or 23, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 3 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
(2) the VH comprises the sequence of SEQ ID NO: 22, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 24, 25, 26, or 27, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
(3) the VH comprises the sequence of SEQ ID NO: 23, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 28 or 29, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VH comprises the sequence of SEQ ID NO: 23, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises the sequence of SEQ ID NO: 28 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto. In certain embodiments, the VH comprises the sequence of SEQ ID NO: 23; and/or, the VL comprises the sequence of SEQ ID NO: 28.

### TIGIT-15, -18, -52, -63, -87, -94, -100

In certain embodiments, the antibody or antigen-binding fragment thereof of the disclosure comprises: HCDR1, HCDR2, HCDR3, LCDR1, LCDR2 and LCDR3 comprising the amino acid sequences of SEQ ID NOs: 118, 119, 120, 121, 122, and 123, respectively.

In certain embodiments, the antibody or antigen-binding fragment thereof of the disclosure comprises: a VH comprising the sequence of SEQ ID NO: 6, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 7, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising the amino acid sequences of SEQ ID NOs: 124, 125, 126, 127, 128, and 129, respectively.

In certain embodiments, the antibody or antigen-binding fragment thereof of the disclosure comprises: a VH comprising the sequence of SEQ ID NO: 8, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 9, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising the amino acid sequences of SEQ ID NOs: 130, 131, 132, 133, 134, and 135, respectively.

In certain embodiments, the antibody or antigen-binding fragment thereof of the disclosure comprises: a VH comprising the sequence of SEQ ID NO: 12, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 13, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising the amino acid sequences of SEQ ID NOs: 136, 137, 138, 139, 140, and 141, respectively.

In certain embodiments, the antibody or antigen-binding fragment thereof of the disclosure comprises: a VH comprising the sequence of SEQ ID NO: 14, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 15, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising the amino acid sequences of SEQ ID NOs: 142, 143, 144, 145, 146 and 147, respectively.

In certain embodiments, the antibody or antigen-binding fragment thereof of the disclosure comprises: a VH comprising the sequence of SEQ ID NO: 16, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 17, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising the amino acid sequences of SEQ ID NOs: 148, 149, 150, 151, 152 and 153, respectively.

In certain embodiments, the antibody or antigen-binding fragment thereof of the disclosure comprises: a VH comprising the sequence of SEQ ID NO: 18, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 19, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising the amino acid sequences of SEQ ID NOs: 154, 155, 156, 157, 158 and 159, respectively.

In certain embodiments, the antibody or antigen-binding fragment thereof of the disclosure comprises: a VH comprising the sequence of SEQ ID NO: 20, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising the sequence of SEQ ID NO: 21, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure as defined in any one of the preceding embodiments can further comprise a heavy chain constant region (CH) comprising an amino acid sequence derived from a human immunoglobulin heavy chain constant region.

In certain embodiments, the heavy chain constant region is an IgG heavy chain constant region, such as an IgG1, IgG2, IgG3 or IgG4 heavy chain constant region. In certain embodiments, the heavy chain constant region comprises the amino acid sequence of SEQ ID NO: 69.

In certain embodiments, the heavy chain constant region has effector function (e.g., ADCC) or enhanced effector function (e.g., ADCC).

In certain embodiments, the antibody or antigen-binding fragment thereof of the disclosure as defined in any one of the preceding embodiments can further comprise a light chain constant region (CL) comprising an amino acid sequence derived from a human immunoglobulin light chain constant region.

In certain embodiments, the light chain constant region is a kappa light chain constant region. In certain embodiments, the light chain constant region comprises the amino acid sequence of SEQ ID NO: 70.

In certain embodiments, the antibody or antigen-binding fragment thereof is derived from TIGIT-6, comprising:
a heavy chain comprising the sequence of SEQ ID NO: 56, 58, 59, 60, or 61, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or,
a light chain comprising the sequence of SEQ ID NO: 57 or 62, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises:
(1) a heavy chain comprising the sequence of SEQ ID NO: 56, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising the sequence of SEQ ID NO: 57, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
(2) a heavy chain comprising the sequence of SEQ ID NO: 58, 59, 60, or 61, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising the sequence of SEQ ID NO: 62, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof is derived from TIGIT-19, comprising:
a heavy chain comprising the sequence of SEQ ID NO: 63, 65, or 66, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or,
a light chain comprising the sequence of SEQ ID NO: 64, 67, or 68, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises:
(1) a heavy chain comprising the sequence of SEQ ID NO: 63, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising the sequence of SEQ ID NO: 64, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
(2) a heavy chain comprising the sequence of SEQ ID NO: 65, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising the sequence of SEQ ID NO: 67 or 68, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
(3) a heavy chain comprising the sequence of SEQ ID NO: 66, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising the sequence of SEQ ID NO: 67 or 68, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof is derived from TIGIT-3, comprising:
a heavy chain comprising the sequence of SEQ ID NO: 52 or 54, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or,
a light chain comprising the sequence of SEQ ID NO: 53 or 55, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises:
(1) a heavy chain comprising the sequence of SEQ ID NO: 52, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising the sequence of SEQ ID NO: 53, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
(2) a heavy chain comprising the sequence of SEQ ID NO: 54, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising the sequence of SEQ ID NO: 55, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure as defined in any one of the preceding embodiments is selected from the group consisting of scFv, Fab, Fab′, (Fab′)₂, Fv fragments, diabodies, bispecific antibodies, multispecific antibodies, chimeric antibodies, and humanized antibodies.

In another aspect, the disclosure provides an immunoconjugate, comprising the antibody or an antigen binding fragment thereof of the disclosure and an effector molecule. In certain embodiments, the effector molecule is a therapeutic agent. In certain embodiments, the therapeutic agent is selected from the group consisting of a drug, a toxin, a radioisotope, a protein, a peptide, and a nucleic acid.

In another aspect, the disclosure provides a bispecific or multispecific antibody, comprising the antibody or an antigen binding fragment thereof of the disclosure. In certain embodiments, the bispecific or multispecific antibody specifically binds to TIGIT (e.g., human TIGIT) and a second target. In certain embodiments, the bispecific or multispecific antibody comprises a first antigen binding domain from the antibody or an antigen binding fragment thereof of the disclosure and a second antigen binding domain from an antibody against a second target. In certain embodiments, the second target is a different immunomodulatory receptor.

In another aspect, the disclosure provides an isolated nucleic acid molecule, comprising a nucleotide sequence encoding the antibody or an antigen binding fragment thereof of the disclosure, or the heavy chain variable region and/or light chain variable region thereof, or the bispecific or multispecific antibody of the disclosure.

In another aspect, the disclosure provides a vector (e.g., a cloning vector or an expression vector), comprising the isolated nucleic acid molecule of the disclosure.

In another aspect, the disclosure provides a host cell, comprising the isolated nucleic acid molecule of the disclosure or the vector of the disclosure.

In another aspect, provided is a method for producing the antibody or antigen-binding fragment thereof of the disclosure, or the bispecific or multispecific antibody of the disclosure, comprising, culturing a host cell comprising the isolated nucleic acid molecule of the disclosure or the vector of the disclosure or the host cell of the disclosure under a condition allowing expression of the antibody or antigen-binding fragment thereof or the bispecific or multispecific antibody, and recovering the antibody or antigen-binding fragment thereof or the bispecific or multispecific antibody from a culture of the cultured host cell.

In another aspect, the disclosure provides a pharmaceutical composition, comprising the antibody or antigen-binding fragment thereof of the disclosure, or the immunoconjugate of the disclosure, or the bispecific or multispecific antibody of the disclosure, or the isolated nucleic acid molecule, vector, or host cell of the disclosure, and a pharmaceutically acceptable carrier and/or excipient. In certain embodiments, the pharmaceutical composition of the disclosure may further comprise an additional therapeutic agent. In certain embodiments, the additional therapeutic agent is an anti-tumor agent. In certain embodiments, the additional therapeutic agent is an additional immune checkpoint inhibitor, such as anti-PD-1 antibody, anti-PD-L1 antibody, anti-TIM-3 antibody, anti-LAG-3 antibody, or anti-CTLA-4 antibody. In certain embodiments, the additional therapeutic agent is a cytotoxic agent, such as an alkylating agent, an anti-mitotic agent, an antitumor antibiotic, an antimetabolite, a topoisomerase inhibitor, a tyrosine kinase inhibitor, or a radionuclide.

In another aspect, provided is use of the antibody or an antigen-binding fragment thereof of the disclosure, or the immunoconjugate of the disclosure, or the bispecific or multispecific antibody of the disclosure, or the isolated nucleic acid molecule, vector, or host cell of the disclosure, or the pharmaceutical composition of the disclosure, in the manufacture of a medicament for use in (a) increasing immune cell activation in response to an antigen in a subject; (b) enhancing an immune response in a subject; (c) reducing or eliminating TIGIT-mediated immune suppression in a subject; (d) reducing or depleting regulatory T cells in a tumor of a subject; (e) treating tumor in a subject; and/or (f) treating an infectious disease in a subject.

In another aspect, provided is the antibody or an antigen-binding fragment thereof of the disclosure, or the immunoconjugate of the disclosure, or the bispecific or multispecific antibody of the disclosure, or the isolated nucleic acid molecule, vector, or host cell of the disclosure, or the pharmaceutical composition of the disclosure, for use in (a) increasing immune cell activation in response to an antigen in a subject; (b) enhancing an immune response in a subject; (c) reducing or eliminating TIGIT-mediated immune suppression in a subject; (d) reducing or depleting regulatory T cells in a tumor of a subject; (e) treating tumor in a subject; and/or (f) treating an infectious disease in a subject.

In another aspect, provided is a method of (a) increasing immune cell activation in response to an antigen in a subject; (b) enhancing an immune response in a subject; (c) reducing or eliminating TIGIT-mediated immune suppression in a subject; (d) reducing or depleting regulatory T cells in a tumor of a subject; (e) treating tumor in a subject; and/or (f) treating an infectious disease in a subject; wherein the method comprising administering to a subject in need thereof a therapeutically effective amount of the antibody or an antigen-binding fragment thereof of the disclosure, or the immunoconjugate of the disclosure, or the bispecific or multispecific antibody of the disclosure, or the isolated nucleic acid molecule, vector, or host cell of the disclosure, or the pharmaceutical composition of the disclosure.

In certain embodiments, the tumor is a solid tumor, e.g., ovarian cancer, endometrial cancer, breast cancer, lung cancer (small cell or non-small cell), colon cancer, prostate cancer, cervical cancer, pancreatic cancer, gastric cancer, esophageal cancer, hepatocellular carcinoma (liver cancer), renal cell carcinoma (kidney cancer), head-and-neck tumors, mesothelioma, melanoma, sarcomas, or brain tumors (e.g., gliomas, such as glioblastomas).

In certain embodiments, the tumor is a hematological malignancy, e.g., leukemia, lymphoma and myeloma, including acute myeloid leukemia, adult T-cell leukemia, T-cell large granula lymphocyte leukemia, acute lymphoblastic leukemia, chronic lymphocytic leukemia, chronic myelogenous leukemia, acute monocytic leukemia, Hodgkin's and Non-Hodgkin's lymphoma and multiple myeloma.

In certain embodiments, the tumor is a metastatic cancer, refractory cancer, or recurrent cancer.

In certain embodiments, the subject is a mammal, such as a human.

In certain embodiments, the antibody or antigen-binding fragment thereof, or the immunoconjugate of the disclosure, or the bispecific or multispecific antibody of the disclosure, or the pharmaceutical composition is used in combination with an additional therapeutic agent or an additional therapy. In certain embodiments, the additional therapeutic agent is an anti-tumor agent. In certain embodiments, the additional therapeutic agent is an additional immune checkpoint inhibitor, such as anti-PD-1 antibody, anti-PD-L1 antibody, anti-TIM-3 antibody, anti-LAG-3 antibody, or anti-CTLA-4 antibody. In certain embodiments, the additional therapeutic agent is a cytotoxic agent, such as an alkylating agent, an anti-mitotic agent, an antitumor antibiotic, an antimetabolite, a topoisomerase inhibitor, a tyrosine kinase inhibitor, or a radionuclide. In certain embodiments, the additional therapy is a standard cancer treatment, such as surgery, chemotherapy, radiation therapy, targeted therapy, immunotherapy, hormone therapy, gene therapy or palliative care.

Further features and advantages of certain embodiments of the present disclosure will become more fully apparent in the following description of the embodiments and drawings thereof, and from the claims.

### BRIEF DESCRIPTION OF THE DRAWINGS

FIG. 1. TIGIT/CD155 blockade reporter assay of chimeric antibodies. Antibodies 4.1D3 and Hu1217-2-2 were used as positive controls.

FIG. 2a. Monovalent binding affinity between human TIGIT and chimeric/humanized anti-TIGIT antibodies. Antibodies 4.1D3 and Hu1217-2-2 were used as positive controls.

FIG. 2b. Monovalent binding affinity between cynomolgus TIGIT and chimeric/humanized anti-TIGIT antibodies. Antibodies 4.1D3 and Hu1217-2-2 were used as positive controls.

FIG. 3a. TIGIT/CD155 blockade reporter assay of TIGIT-3 humanized antibodies. Antibodies 4.1D3 and Hu1217-2-2 were used as positive controls.

FIG. 3b. TIGIT/CD155 blockade reporter assay of TIGIT-6 humanized antibodies. Antibodies 4.1D3 and Hu1217-2-2 were used as positive controls.

FIG. 3c. TIGIT/CD155 blockade reporter assay of TIGIT-19 humanized antibodies. Antibodies 4.1D3 and Hu1217-2-2 were used as positive controls.

FIG. 4a. Activation of CMV-specific human primary T cells by TIGIT-3 chimeric/humanized antibodies. Antibodies 4.1D3 and Hu1217-2-2 were used as positive controls.

FIG. 4b. Activation of CMV-specific human primary T cells by TIGIT-6 chimeric/humanized antibodies. Antibodies 4.1D3 and Hu1217-2-2 were used as positive controls.

FIG. 4c. Activation of CMV-specific human primary T cells by TIGIT-19 chimeric/humanized antibodies. Antibodies 4.1D3 and Hu1217-2-2 were used as positive controls.

FIG. 5a. Killing of human primary total T cells (CD3+) through ADCC by chimeric and humanized anti-TIGIT antibodies. Anti-CD3 IgG1 Hu38E4 used as positive controls.

FIG. 5b. Killing of human primary total T helper cells (CD4+) through ADCC by chimeric and humanized anti-TIGIT antibodies. Anti-CD3 IgG1 Hu38E4 used as positive controls.

FIG. 5c. Killing of human primary total cytotoxic T cells (CD8+) through ADCC by chimeric and humanized anti-TIGIT antibodies. Anti-CD3 IgG1 Hu38E4 used as positive controls.

FIG. 5d. Killing of human primary Treg cells (FOXP3+CD4+) through ADCC by chimeric and humanized anti-TIGIT antibodies. Anti-CD3 IgG1 Hu38E4 used as positive controls.

FIG. 6. In vivo efficacy of humanized anti-TIGIT antibodies. FIG. 6a shows the inhibition of tumor growth by humanized anti-TIGIT antibodies. FIG. 6b shows the changes of body weight of mice.

### DETAILED DESCRIPTION

### Definitions

In the present disclosure, the scientific and technical terms used herein have the meanings commonly understood by those skilled in the art, unless otherwise stated. For better understanding of the present disclosure, definitions and explanations of terms are provided below.

As used herein, the term “TIGIT” refers to T-cell immunoreceptor with Ig and ITIM domains, a member of the PVR (poliovirus receptor) family of immunoglobin proteins, which binds to PVR/CD155 and Nectin-2/CD112. TIGIT is also referred to as TIGIT, WUCAM, Vstm3 and Vsig9. “human TIGIT” refers to a TIGIT protein encoded by a wild-type human TIGIT gene. An exemplary amino acid sequence of human TIGIT protein is provided as SEQ ID NO: 1.

As used herein, the term “antibody” refers to an immunoglobulin molecule capable of specific binding to a target (such as a carbohydrate, polynucleotide, lipid, polypeptide, etc.) through at least one antigen recognition site, located in the variable region of the immunoglobulin molecule. Unless otherwise indicated or clear from the context, the term “antibody” as used to herein may include whole antibodies and any antigen binding fragments (i.e., “antigen-binding portions”) or single chains thereof. In one embodiment, “antibody” is typically composed of two pairs of polypeptide chains (each pair has a “light” (L) chain and a “heavy” (H) chain). Antibody light chains can be classified as κ and λ light chains. Heavy chains can be classified as μ, δ, γ, α, or ε, and the isotypes of antibody are defined as IgM, IgD, IgG, IgA, and IgE, respectively. Within the light and heavy chains, variable region and constant region are joined by a “J” region of about 12 or more amino acids, and the heavy chain further comprises a “D” region of about 3 or more amino acids. Each heavy chain consists of a heavy chain variable region (V_{H}) and a heavy chain constant region (C_{H}). The heavy chain constant region consists of three domains (C_{H}1, C_{H}2 and C_{H}3). Each light chain consists of a light chain variable region (V_{L}) and a light chain constant region (C_{L}). The light chain constant region consists of one domain C_{L}. The V_{H} and V_{L} regions can also be subdivided into hypervariable regions (referred to as complementarity determining regions (CDRs)) interspaced with relatively conservative regions called framework regions (FR). Each of V_{H} and V_{L} consists of three CDRs and four FRs arranged in the following order: FR1, CDR1, FR2, CDR2. FR3, CDR3, FR4 from the amino terminus to the carboxy terminus. The variable regions (V_{H} and V_{L}) of each heavy/light chain pair form an antibody binding site, respectively. The term “antibody” is not limited by any particular method for producing the antibody, for example, it comprises recombinant antibodies, monoclonal antibodies, and polyclonal antibodies.

As used herein, the term “complementarity determining region” or “CDR” refers to the amino acid residues in the variable region of an antibody that are responsible for antigen binding. The precise boundaries of these amino acid residues can be defined according to various numbering systems known in the art, for example, according to the definitions in the Kabat numbering system (Kabat et al., Sequences of Proteins of Immunological Interest, 5th Ed. Public Health Service, National Institutes of Health, Bethesda, Md., 1991), Chothia numbering system (Chothia & Lesk (1987) J. Mol. Biol. 196: 901-917; Chothia et al. (1989) Nature 342: 878-883), AbM numbering system (Martin, in Antibody Engineering, Vol. 2, Chapter 3, Springer Verlag) or IMGT numbering system (Lefranc et al., Dev. Comparat. Immunol. 27: 55-77, 2003). For a given antibody, those skilled in the art can easily identify the CDRs defined by each numbering system. Moreover, the correspondence between different numbering systems is well known to those skilled in the art (e.g., see Lefranc et al., Dev. Comparat. Immunol. 27: 55-77, 2003). In certain embodiments, the CDRs of the antibodies of the disclosure are defined according to Kabat, AbM, IMGT, or Chothia numbering system, or any combination thereof. Unless otherwise indicated or clear from the context, the CDRs of the antibodies of the disclosure are preferably defined according to Abm numbering system (for HCDR1) and Kabat numbering system (for HCDR2 and 3, LCDR1-3).

As used herein, the term “framework region” or “FR” residues refers to those amino acid residues other than the CDR residues as defined above in the variable regions of antibody.

As used herein, the term “antigen-binding fragment” of an antibody refers to a polypeptide comprising a fragment of a full-length antibody, which retains the ability to specifically bind to the same antigen to which the full-length antibody binds, and/or competes with the full-length antibody to specifically bind to the antigen, and which is also referred to as an “antigen-binding portion”. An antigen-binding fragment of an antibody can be produced by recombinant DNA techniques or by enzymatic or chemical cleavage of the intact antibody. In certain embodiments, the antigen-binding fragment comprises Fab, Fab′, F(ab′)₂, Fd, Fv, dAb and fragments of complementarity determining regions (CDRs), single chain antibodies (e.g., scFv), chimeric antibodies, diabodies, and polypeptides comprising at least a portion of the antibody that is sufficient to confer the specific antigen binding ability to the polypeptide.

As used herein, the term “Fd fragment” refers to an antibody fragment consisting of V_{H} and C_{H}1 domains; the term “dAb fragment” refers to an antibody fragment consisting of V_{H} domain; the term “Fab fragment” refers to an antibody fragment consisting of V_{L}, V_{H}, C_{L} and C_{H}1 domains; the term “F(ab′)₂ fragment” refers to an antibody fragment comprising two Fab fragments linked by a disulfide bridge at the hinge region.

As used herein, the term “Fv fragment” refers to an antibody fragment consisting of V_{L} and V_{H} domains of a single arm of an antibody. An Fv fragment is generally considered to be the smallest antibody fragment that can form a complete antigen binding site. It is believed that six CDRs confer the antigen binding specificity to the antibody. However, even one variable region (e.g., an Fd fragment, which contains only three CDRs specific for an antigen) is able to recognize and bind an antigen, albeit with less affinity than that of the entire binding site.

As used herein, the term “scFv” refers to a single polypeptide chain comprising VL and VH domains, having a general structure of NH₂-VL-linker-VH—COOH or NH₂-VH-linker-VL-COOH. A suitable linker of prior art consists of a repeated GGGGS amino acid sequence or variants thereof. For example, a linker having amino acid sequence (GGGGS)₄ can be used, but variants thereof can also be used. In some cases, there may also be a disulfide bond between the VH and VL of scFv.

As used herein, the term “diabody” refers to a dimer of scFv which consists of VH and VL domains connected by a short peptide linker. The linker is too short to form intrachain pairing of VH and VL domains. Instead, two such scFv fragments are co-expressed to form multimers by inter-chain pairing (cross-over pairing) of VH and VL domains.

Each of the antigen-binding fragments maintains the ability to specifically bind to the same antigen to which the full-length antibody binds, and/or compete with the full-length antibody for specific binding to the antigen. An antigen-binding fragment can be obtained from a given antibody (e.g., an intact antibody provided herein) using conventional techniques known to those skilled in the art (e.g., recombinant DNA techniques or enzymatic or chemical cleavage methods), and can be screened for specificity in the same manner by which intact antibodies are screened.

As used herein, the terms “mAb” and “monoclonal antibody” refer to an antibody or a fragment of an antibody from a population of highly homologous antibody molecules, i.e., a population of completely identical antibody molecules except for natural mutation that may occur spontaneously. A monoclonal antibody has a high specificity for a single epitope of an antigen. Polyclonal antibody, relative to monoclonal antibody, generally comprises at least two or more different antibodies which generally recognize different epitopes on an antigen.

Monoclonal antibodies can be obtained by hybridoma technique which relies on fusing B cells from an immunized animal (e.g., mouse) with immortal myeloma cells and growing the cells under conditions in which only the fused cells can survive. The resultant fused cells that grow out are called hybridomas. Each hybridoma makes only one immunoglobulin, derived from one B cell from the immunized animal. The antibodies secreted by many hybridoma clones are screened for binding to the antigen of interest, and the clone with the desired specificity is selected and expanded. The products of these individual clones are monoclonal antibodies, and each monoclonal antibody is specific for a single epitope on the antigen used to immunize the animal.

Monoclonal antibodies can also be obtained by genetic engineering recombinant techniques. A DNA molecule encoding the heavy chain and light chain genes of a monoclonal antibody can be isolated from a hybridoma cell by PCR amplification using a primer that is specific to the genes. The obtained DNA molecule is inserted into an expression vector, and then transfected into a host cell (such as E. coli cells, COS cells, CHO cells, or other myeloma cells that do not produce immunoglobulin), and cultured under appropriate conditions, thereby obtaining the desired recombinant antibody.

As used herein, the term “murine antibody” refers to an antibody that is obtained by hybridoma technique by the fusion of B cells from immunized mouse with immortal myeloma cells, followed by screening and purification; or an antibody that is secreted by plasma cells which is formed by differentiation and proliferation of B cells after antigen invades the mouse.

As used herein, the term “chimeric antibody” refers to such an antibody wherein a part of its light chain and/or heavy chain is derived from an antibody (which may be originated from a specific species or belongs to a specific antibody type or subtype), and the other part of its light chain and/or heavy chain is derived from another antibody (which may be originated from an identical or different species or belongs to an identical or different antibody type or subtype), provided that the antibody still retains the activity of binding to the antigen of interest. For example, the term “chimeric antibody” may include such an antibody (e.g., a human-mouse chimeric antibody), wherein the heavy chain and light chain variable region of the antibody are from a first antibody (e.g., a mouse antibody), while the heavy chain and light chain constant region of the antibody are from a second antibody (e.g., a human antibody).

The chimeric antibody of the present disclosure can be prepared based on the sequence of the murine monoclonal antibody prepared above. The DNA encoding the heavy and light chains can be obtained from the target murine hybridoma and engineered using standard molecular biology techniques to contain non-mouse (e.g., human) immunoglobulin sequences. To prepare a chimeric antibody, a variable region of murine immunoglobulin can be linked to a constant region of human immunoglobulin using a method known in the art. For example, a DNA encoding VH is operably linked to another DNA molecule encoding heavy chain constant region so as to obtain a full-length heavy chain gene. The sequence of human heavy chain constant region gene is known in the art (see, for example, Kabat, E A et al. (1991) Sequences of Proteins of Immunological Interest, Fifth Edition, U.S. Department of Health and Human Services, NIH Publication No. 91-3242), a DNA fragment containing these regions can be obtained by standard PCR amplification. The heavy chain constant region may be an IgG1, IgG2, IgG3, IgG4, IgA, IgE, IgM or IgD constant region, but is generally preferably an IgG (e.g., IgG1 or IgG4) constant region. For example, a DNA encoding VL is operably linked to another DNA molecule encoding light chain constant region CL so as to obtain a full-length light chain gene (as well as a Fab light chain gene). The sequence of human light chain constant region gene is known in the art (see, for example, Kabat, E A et al. (1991) Sequences of Proteins of Immunological Interest, Fifth Edition, US Department of Health and Human Services, NIH Publication No. 91-3242), a DNA fragment containing these regions can be obtained by standard PCR amplification. The light chain constant region may be a κ or λ constant region but is generally preferably a κ constant region.

As used herein, the term “humanized antibody” refers to a genetically engineered non-human antibody of which the amino acid sequence has been modified to increase its homology to the sequence of a human antibody. Generally, all or part of the CDR regions of a humanized antibody are derived from a non-human antibody (donor antibody), and all or part of the non-CDR regions (e.g., variable region FR and/or constant region) are derived from a human immunoglobulin (receptor antibody). The humanized antibody typically retains the expected properties of the donor antibody, including, but not limited to, the ability of specifically binding to TIGIT (e.g., human TIGIT), the ability of blocking binding to PVR/CD155, and the like. The donor antibody can be an antibody of mouse, rat, rabbit, or non-human primate (e.g., cynomolgus monkey) that has the expected properties.

The humanized antibody of the present disclosure can be prepared based on the sequence of the murine monoclonal antibody prepared above. The DNA encoding the heavy and light chains can be obtained from the target murine hybridoma and engineered using standard molecular biology techniques to contain non-mouse (e.g., human) immunoglobulin sequences. To prepare a humanized antibody, murine CDR regions can be grafted onto a human framework sequence by using any methods known in the art. In case of affinity loss of these straight-graft antibodies, several framework residues can be mutated back to their murine counterparts (i.e., backmutation) to restore the binding affinity of antibody. In the meantime, some CDR residues can also be either mutated to their human counterparts to increase the humanness of the antibody or to some other residues to remove potential post-translational modification spot or both.

In the context, the expected properties of the antibodies of the present disclosure comprise at least one of the following: (a) the ability to specifically bind to TIGIT (e.g., human TIGIT); (b) the ability to block binding of TIGIT to PVR/CD155; (c) reducing or eliminating TIGIT-mediated immune suppression; (d) reducing or depleting regulatory T cells, for example, in a tumor; (e) the ability to decrease or inhibite Treg activity; (f) increasing immune cell activation; (g) enhancing an immune response; (h) treating tumor; and/or (i) treating an infectious disease.

As used herein, the term “bispecific antibody” refers to an artificial hybrid antibody having two different heavy/light chain pairs, giving rise to two antigen binding sites with specificity for different antigens. Bispecific antibodies can be produced by a variety of methods, including linking of a first antibody or its fragment and a second antibody or its fragment, for example, by chemical coupling, gene fusion, non-covalent association, or other means. “Multispecific antibody” refers to an artificial hybrid antibody that has more than two different binding specificities, including for example, trispecific antibody or tetraspecific antibody.

As used herein, the term “specifically bind” or “specifically binding” refers to the binding of two molecules in a non-random manner, such as the reaction between an antibody and the antigen it directs to. In some embodiments, an antibody that specifically binds to an antigen (or an antibody specific for an antigen) refers to an antibody that binds to the antigen with an affinity (K_{D}) of less than about 10⁻⁵ M. e.g., less than about 10⁻⁶ M, 10⁻⁷ M, 10⁻⁸ M, 10⁻⁹ M, or 10⁻¹⁰ M or less.

As used herein, the term “K_{D}” refers to a dissociation constant of a specific antibody-antigen interaction, which is used to describe the binding affinity of an antibody to an antigen. The smaller the dissociation constant, the more tightly bound the antibody is, and the higher the affinity between antibody and antigen. Generally, an antibody (e.g., the antibody of the disclosure) binds to an antigen (e.g., human TIGIT) with a K_{D} of less than about 10⁻⁵ M, e.g., less than about 10⁻⁶ M, 10⁻⁷ M, 10⁻⁸ M, 10⁻⁹ M, or 10⁻¹⁰ M or less, determined by, for example, surface plasmon resonance (SPR) in BIACORE device.

As used herein, the term “vector” refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element, and a reporter gene. In addition, a vector may comprise origin of replication.

As used herein, the term “host cell” refers to a cell into which a vector can be introduced or transformed, including, but not limited to, prokaryotic cells such as E. coli or Bacillus subtilis, and eukaryotic cells such as mammal cells (e.g., mouse cell or human cell), insect cells or yeast cells. Suitable eukaryotic cells include, but not limited to, NS0 cells, Vero cells, Hela cells, COS cells, CHO cells, HEK293 cells, BHK cells or MDCKII cells.

As used herein, the term “identity” refers to the match degree between two polypeptides or between two nucleic acids. When two sequences for comparison have the same monomer sub-unit of base or amino acid at a certain site (e.g., each of two DNA molecules has an adenine at a certain site, or each of two polypeptides has a lysine at a certain site), the two molecules are identical at the site. The percent identity between two sequences is a function of the number of identical sites shared by the two sequences over the total number of sites for comparison×100. For example, if 6 of 10 sites of two sequences are matched, these two sequences have an identity of 60%. For example, DNA sequences: CTGACT and CAGGTT share an identity of 50% (3 of 6 sites are matched). Generally, the comparison of two sequences is conducted in a manner to produce maximum identity. Such alignment can be conducted by using a computer program such as Align program (DNAstar, Inc.) which is based on the method of Needleman, et al. (J. Mol. Biol. 48:443-453, 1970). The percent identity between two amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, the term “pharmaceutically acceptable carrier and/or excipient” refers to a carrier and/or excipient that is pharmacologically and/or physiologically compatible with a subject and an active ingredient, which is well known in the art (see, for example, Remington's Pharmaceutical Sciences. Edited by Gennaro A R, 19th ed. Pennsylvania: Mack Publishing Company, 1995) and includes, but is not limited to: pH adjusting agents, surfactants, adjuvants, ionic strength enhancers, diluents, agents to maintain osmotic pressure, agents to delay absorption, preservatives. For example, pH adjusting agents include, but are not limited to, phosphate buffered saline. Surfactants include, but are not limited to, cationic, anionic or non-ionic surfactants, such as Tween-80). Ionic strength enhancers include, but are not limited to, sodium chloride. Preservatives include, but are not limited to, various antibacterial and antifungal agents, such as parabens, trichloro-t-butanol, phenol, sorbic acid, and the like. Agents to maintain osmotic pressure include, but are not limited to, sugar, NaCl, and the like. Agents to delay absorption include, but are not limited to, monostearate and gelatin. Diluents include, but are not limited to, water, aqueous buffers (e.g., buffered saline), alcohols and polyols (e.g., glycerol), and the like. Preservatives include, but are not limited to, various antibacterial and antifungal agents, such as thimerosal, 2-phenoxyethanol, parabens, trichloro-t-butanol, phenol, sorbic acid, and the like. Stabilizers have the meaning commonly understood by those skilled in the art, which can stabilize the desired activity of active ingredient in drug, including but not limited to sodium glutamate, gelatin, SPGA, saccharides (e.g., sorbitol, mannitol, starch, sucrose, lactose, dextran, or glucose), amino acids (e.g., glutamic acid, glycine), proteins (e.g., dried whey, albumin, or casein) or degradation products thereof (e.g., lactalbumin hydrolysate), etc. In certain exemplary embodiments, the pharmaceutically acceptable carrier or excipient includes a sterile injectable liquid (e.g., an aqueous or non-aqueous suspension or solution). In certain exemplary embodiments, such sterile injectable liquid is selected from the group consisting of water for injection (WFI), bacteriostatic water for injection (BWFI), sodium chloride solution (e.g., 0.9% (w/v) NaCl), glucose solution (e.g., 5% glucose), surfactant-containing solution (e.g., 0.01% polysorbate 20), pH buffer solution (e.g., phosphate buffer solution), Ringer's solution, and any combination thereof.

As used herein, the term “treatment/treating” refers to a method that is carried out in order to obtain a beneficial or desired clinical outcome. For the purpose of the disclosure, the beneficial or desired clinical outcome includes, but is not limited to, easing symptom, narrowing the scope of disease, stabilizing (i.e., not aggravating) the state of disease, delaying or slowing the progress of disease, and alleviating symptoms (either partially or completely), no matter detectable or not detectable. In addition, “treatment” also refers to a prolonged survival period compared to the expected survival period (if no treatment is accepted). In certain embodiments, the beneficial or desired clinical outcome described herein includes, but is not limited to, slowing of tumor progression, cancer regression, enhancement of anti-tumor immune response, a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient.

As used herein, the term “subject” refers to any human or non-human animal that receives either prophylactic or therapeutic treatment. The term “non-human animal” includes all vertebrates, e.g., mammals and non-mammals, such as non-human primates, sheep, dog, cow; chickens, amphibians, reptiles, etc.

As used herein, the term “an effective amount” refers to an amount that is sufficient to achieve or at least partially achieve the expected effect. For example, an effective amount for treating a disease refers to an amount effective for curing or at least partially blocking a disease and its complication in a patient having the disease. The determination of such an effective amount is within the ability of a person skilled in the art. For example, an amount effective for a therapeutic use depends on severity of a disease to be treated, general state of the immune system in a patient, general conditions of a patient, such as age, weight and gender, administration routes of drugs, additional therapies used simultaneously, and the like.

As used herein, the term “immune cell” includes cells that have a hematopoietic origin and play a role in immune response, for example, lymphocytes, such as B cells and T cells; natural killer (NK) cells; myeloid cells, such as monocytes, macrophages, eosinophils, mast cells, basophils and granulocytes.

As used herein, the term “immune response” refers to an effect of immune cells (e.g., lymphocytes, antigen-presenting cells, phagocytes, or granulocytes) and soluble macromolecules (including antibodies, cytokines, and complements) produced by the immune cells or liver, which leads to selective damage or destruction of invasive pathogens, cells or tissues infected with pathogens, cancer cells, or normal human cells or tissues in the context of autoimmune or pathological inflammation, or removal of them from the body. In the present disclosure, the term “antigen-specific T cell response” refers to an immune response produced by a T cell, which is generated when the T cell is stimulated by the T cell specific antigen. Non-limiting examples of response produced by T cell upon antigen-specific stimulation include the proliferation of T cell and the production of cytokine such as IFN-γ and/or IL-2.

As used herein, the term “effector function” refers to those biological activities attributable to the Fc region of an antibody (Fc region of a natural sequence or an amino acid sequence variant), which varies as the isotype of an antibody. Examples of antibody effector functions include, but are not limited to, Fc receptor binding affinity, antibody-dependent cell-mediated cytotoxicity (ADCC), complement dependent cytotoxicity (CDC), antibody-dependent cellular phagocytosis (ADCP), downregulation of cell surface receptors (e.g., B cell receptors), B cell activation, cytokine secretion, half-life/clearance of antibodies and antigen-antibody complexes, and the like. Methods for altering the effector function of an antibody are known in the art, for example by introducing a mutation in the Fc region.

As used herein, the term “antibody-dependent cell-mediated cytotoxicity (ADCC)” refers to a form of cytotoxicity, in which cytotoxic effector cells specifically bind to the target cells to which the antigen is attached, through the binding of Ig to an Fc receptor (FcR) presented on cytotoxic cells (e.g., natural killer (NK) cells, neutrophils or macrophages), and then kills the target cells by secreting cytotoxins. Methods for detecting ADCC activity of an antibody are known in the art and can be evaluated, for example, via the flow cytometry-based ADCC assay as described in EXAMPLE 5.

As used herein, the terms “cancer” and “tumor” are used interchangeably and refer to a large group of diseases characterized by the uncontrolled growth of abnormal cells in the body. Unregulated cell division may lead to the formation of malignant tumors or cells that invade adjacent tissues, and may metastasize to distant parts of the body through the lymphatic system or bloodstream. Cancers include benign and malignant cancers as well as dormant tumors or micrometastasis. Cancers also include hematological malignancies.

As used herein, the terms “about” when used to modify a numeric value or numeric range, indicate that deviations of up to 10% above (e.g., up to 5% above, up to 2% above, or up to 1% above) and up to 10% below (e.g., up to 5% below, up to 2% below, or up to 1% below) the value or range remain within the intended meaning of the recited value or range.

### Antibodies of the Disclosure

In one aspect, the disclosure provides an antibody or antigen-binding fragment thereof, that binds to TIGIT (e.g., human TIGIT), comprising:
(i) HCDR1 comprising the sequence of SEQ ID NO: 84-86, 98-100, 71, 118, 124, 130, 136, 142, 148, or 154, e.g., the sequence of SEQ ID NO: 84-85, 98-99, 71, 118, 124, 130, 136, 142, 148, or 154;
(ii) HCDR2 comprising the sequence of SEQ ID NO: 87-91, 101-107, 72-74, 119, 125, 131, 137, 143, 149, or 155, e.g., the sequence of SEQ ID NO: 87-90, 101-106, 72-73, 119, 125, 131, 137, 143, 149, or 155;
(iii) HCDR3 comprising the sequence of SEQ ID NO: 92, 108, 75, 120, 126, 132, 138, 144, 150, or 156;
(iv) LCDR1 comprising the sequence of SEQ ID NO: 93-95, 109-113, 76-78, 121, 127, 133, 139, 145, 151, or 157, e.g., the sequence of SEQ ID NO: 93-94, 109-112, 76-77, 121, 127, 133, 139, 145, 151, or 157;
(v) LCDR2 comprising the sequence of SEQ ID NO: 96, 114-116, 79-82, 122, 128, 134, 140, 146, 152, or 158, e.g., the sequence of SEQ ID NO: 96, 114-115, 79-81, 122, 128, 134, 140, 146, 152, or 158; and
(vi) LCDR3 comprising the sequence of SEQ ID NO: 97, 117, 83, 123, 129, 135, 141, 147, 153, or 159.

### TIGIT-6

In certain embodiments, the antibody or antigen-binding fragment thereof of the disclosure is derived from murine antibody TIGIT-6 (having the VH of SEQ ID NO: 4 and the VL of SEQ ID NO: 5).

In certain embodiments, the antibody or antigen-binding fragment thereof comprises:
(i) HCDR1 comprising or consisting of the sequence of GYTX₁TENX₂MH (SEQ ID NO: 86), wherein X₁ is I or F, X₂ is T or A;
(ii) HCDR2 comprising or consisting of the sequence of GINPNX₃X₄GTSYX₅QX₆FX₇G (SEQ ID NO: 91), wherein X₃ is N or Q, X₄ is G or A, X₅ is N or S, X₆ is Q or K, X₇ is K or Q;
(iii) HCDR3 comprising or consisting of the sequence of SEQ ID NO: 92;
(iv) LCDR1 comprising or consisting of the sequence of X₈ASQDX₉KTALA (SEQ ID NO: 95), wherein X₈ is K or Q, X₉ is V or I;
(v) LCDR2 comprising or consisting of the sequence of SEQ ID NO: 96; and
(vi) LCDR3 comprising or consisting of the sequence of SEQ ID NO: 97.

In certain embodiments, the HCDR1 comprises or consisting of the sequence of SEQ ID NO: 84 or 85. In certain embodiments, the HCDR2 comprises or consisting of the sequence of SEQ ID NO: 87, 88, 89, or 90. In certain embodiments, the LCDR1 comprises or consisting of the sequence of SEQ ID NO: 93 or 94.

In certain embodiments, the antibody or antigen-binding fragment thereof comprises: HCDR1, HCDR2 and HCDR3 of the VH as set forth in SEQ ID NO: 4, 30, 31, 33, 34, 36, 37, 38, or 39; and LCDR1, LCDR2 and LCDR3 of the VL as set forth in SEQ ID NO: 5, 32, or 35. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof comprises: HCDR1, HCDR2 and HCDR3 of the VH as set forth in SEQ ID NO: 4; and LCDR1, LCDR2 and LCDR3 of the VL as set forth in SEQ ID NO: 5.

In certain embodiments, the antibody or antigen-binding fragment thereof comprises: HCDR1, HCDR2 and HCDR3 of the VH as set forth in SEQ ID NO: 30, 31, 33, 34, or 36; and LCDR1, LCDR2 and LCDR3 of the VL as set forth in SEQ ID NO: 32.

In certain embodiments, the antibody or antigen-binding fragment thereof comprises: HCDR1, HCDR2 and HCDR3 of the VH as set forth in SEQ ID NO: 31, 34, 36, 37, 38, or 39; and LCDR1, LCDR2 and LCDR3 of the VL as set forth in SEQ ID NO: 35.

In certain embodiments, the antibody or antigen-binding fragment thereof comprises: HCDR1, HCDR2 and HCDR3 of the VH as set forth in SEQ ID NO: 36, 37, 38, or 39; and LCDR1, LCDR2 and LCDR3 of the VL as set forth in SEQ ID NO: 35.

In certain embodiments, the antibody or antigen-binding fragment thereof comprises:
(a) HCDR1, HCDR2 and HCDR3 of the VH as set forth in SEQ ID NO: 36; and LCDR1, LCDR2 and LCDR3 of the VL as set forth in SEQ ID NO: 35;
(b) HCDR1, HCDR2 and HCDR3 of the VH as set forth in SEQ ID NO: 37; and LCDR1, LCDR2 and LCDR3 of the VL as set forth in SEQ ID NO: 35;
(c) HCDR1, HCDR2 and HCDR3 of the VH as set forth in SEQ ID NO: 38; and LCDR1, LCDR2 and LCDR3 of the VL as set forth in SEQ ID NO: 35; or
(d) HCDR1, HCDR2 and HCDR3 of the VH as set forth in SEQ ID NO: 39; and LCDR1, LCDR2 and LCDR3 of the VL as set forth in SEQ ID NO: 35.

In certain embodiments, the antibody or antigen-binding fragment thereof comprises:
HCDR1, HCDR2 and HCDR3 comprising or consisting of the amino acid sequences of SEQ ID NOs: 84, 87, and 92, respectively; and LCDR1, LCDR2 and LCDR3 comprising or consisting of the amino acid sequences of SEQ ID NOs: 93, 96, and 97, respectively; or,
HCDR1, HCDR2 and HCDR3 comprising or consisting of the amino acid sequences of SEQ ID NOs: 85, 87, and 92, respectively; and LCDR1, LCDR2 and LCDR3 comprising or consisting of the amino acid sequences of SEQ ID NOs: 93, 96, and 97, respectively; or,
HCDR1, HCDR2 and HCDR3 comprising or consisting of the amino acid sequences of SEQ ID NOs: 84, 88, and 92, respectively; and LCDR1, LCDR2 and LCDR3 comprising or consisting of the amino acid sequences of SEQ ID NOs: 93, 96, and 97, respectively; or,
HCDR1, HCDR2 and HCDR3 comprising or consisting of the amino acid sequences of SEQ ID NOs: 84, 89, and 92, respectively; and LCDR1, LCDR2 and LCDR3 comprising or consisting of the amino acid sequences of SEQ ID NOs: 93, 96, and 97, respectively; or,
HCDR1, HCDR2 and HCDR3 comprising or consisting of the amino acid sequences of SEQ ID NOs: 84, 87, and 92, respectively; and LCDR1, LCDR2 and LCDR3 comprising or consisting of the amino acid sequences of SEQ ID NOs: 94, 96, and 97, respectively; or,
HCDR1, HCDR2 and HCDR3 comprising or consisting of the amino acid sequences of SEQ ID NOs: 84, 88, and 92, respectively; and LCDR1, LCDR2 and LCDR3 comprising or consisting of the amino acid sequences of SEQ ID NOs: 94, 96, and 97, respectively; or,
HCDR1, HCDR2 and HCDR3 comprising or consisting of the amino acid sequences of SEQ ID NOs: 84, 89, and 92, respectively; and LCDR1, LCDR2 and LCDR3 comprising or consisting of the amino acid sequences of SEQ ID NOs: 94, 96, and 97, respectively; or,
HCDR1, HCDR2 and HCDR3 comprising or consisting of the amino acid sequences of SEQ ID NOs: 84, 90, and 92, respectively; and LCDR1, LCDR2 and LCDR3 comprising or consisting of the amino acid sequences of SEQ ID NOs: 94, 96, and 97, respectively; or,
HCDR1, HCDR2 and HCDR3 comprising or consisting of the amino acid sequences of SEQ ID NOs: 85, 89, and 92, respectively; and LCDR1, LCDR2 and LCDR3 comprising or consisting of the amino acid sequences of SEQ ID NOs: 94, 96, and 97, respectively; or,
HCDR1, HCDR2 and HCDR3 comprising or consisting of the amino acid sequences of SEQ ID NOs: 85, 90, and 92, respectively; and LCDR1, LCDR2 and LCDR3 comprising or consisting of the amino acid sequences of SEQ ID NOs: 94, 96, and 97, respectively.

In certain embodiments, the antibody or antigen-binding fragment thereof comprises a heavy chain variable region (VH) and a light chain variable region (VL), wherein: the VH comprises or consists of the sequence of SEQ ID NO: 4 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 5 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto. In certain embodiments, the VH comprises or consists of the sequence of SEQ ID NO: 4; and/or, the VL comprises or consists of the sequence of SEQ ID NO: 5.

In certain embodiments, the antibody or antigen-binding fragment thereof comprises a heavy chain variable region (VH) and a light chain variable region (VL), wherein: the VH comprises framework regions (FRs) derived from a human immunoglobulin heavy chain variable region sequence of GenBank: QVG74320; and/or, the VL comprises framework regions (FRs) derived from a human immunoglobulin light chain variable region sequence of GenBank: ACY78416.

In certain embodiments, the VH comprises or consists of the sequence of SEQ ID NO: 30, 31, 33, 34, 36, 37, 38, or 39, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VL comprises or consists of the sequence of SEQ ID NO: 32 or 35, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises a heavy chain variable region (VH) and a light chain variable region (VL), wherein:
the VH comprises or consists of the sequence of SEQ ID NO: 30, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 32 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 31, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 32 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 33, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 32 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 34, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 32 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 36, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 32 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 31, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 35 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 34, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 35 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 36, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 35 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 37, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 35 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 38, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 35 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or
the VH comprises or consists of the sequence of SEQ ID NO: 39, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 35 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, (a) the VH comprises the sequence of SEQ ID NO: 36; and/or, the VL comprises the sequence of SEQ ID NO: 35; (b) the VH comprises the sequence of SEQ ID NO: 37; and/or, the VL comprises the sequence of SEQ ID NO: 35; (c) the VH comprises the sequence of SEQ ID NO: 38; and/or, the VL comprises the sequence of SEQ ID NO: 35; or (d) the VH comprises the sequence of SEQ ID NO: 39; and/or, the VL comprises the sequence of SEQ ID NO: 35.

### TIGIT-19

In certain embodiments, the antibody or antigen-binding fragment thereof of the disclosure is derived from murine antibody TIGIT-19 (having the VH of SEQ ID NO: 10 and the VL of SEQ ID NO: 11).

In certain embodiments, the antibody or antigen-binding fragment thereof comprises:
(i) HCDR1 comprising or consisting of the sequence of GYX₁FX₂RYSMY (SEQ ID NO: 100), wherein X₁ is A or T, X₂ is S or T;
(ii) HCDR2 comprising or consisting of the sequence of FIDX₃YX₄GGSTYX₅QX₆FX₇G (SEQ ID NO: 107), wherein X₃ is P or A, X₄ is N or S, X₅ is N or A, X₆ is R or K, X₇ is R or Q;
(iii) HCDR3 comprising or consisting of the sequence of SEQ ID NO: 108;
(iv) LCDR1 comprising or consisting of the sequence of RX₈SX₉X₁₀IYX₁₁YLS (SEQ ID NO: 113), wherein X₈ is P or A, X₉ is E or Q, X₁₀ is N or S, X₁₁ is T or S;
(v) LCDR2 comprising or consisting of the sequence of NAKX₁₂LPX₁₃ (SEQ ID NO: 116), wherein X₁₂ is T or S, X₁₃ is E or S; and
(vi) LCDR3 comprising or consisting of the sequence of SEQ ID NO: 117.

In certain embodiments, the HCDR1 comprises or consists of the sequence of SEQ ID NO: 98 or 99. In certain embodiments, the HCDR2 comprises or consists of the sequence of SEQ ID NO: 101, 102, 103, 104, 105, or 106. In certain embodiments, the LCDR1 comprises or consists of the sequence of SEQ ID NO: 109, 110, 111, or 112. In certain embodiments, the LCDR2 comprises or consists of the sequence of SEQ ID NO: 114 or 115.

In certain embodiments, the antibody or antigen-binding fragment thereof comprises: HCDR1, HCDR2 and HCDR3 of the VH as set forth in SEQ ID NO: 10, 40, 46, 47, 48, 49, 50, or 51; and LCDR1, LCDR2 and LCDR3 of the VL as set forth in SEQ ID NO: 11, 41, 42, 43, 44, or 45. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof comprises: HCDR1, HCDR2 and HCDR3 of the VH as set forth in SEQ ID NO: 10; and LCDR1, LCDR2 and LCDR3 of the VL as set forth in SEQ ID NO: 11.

In certain embodiments, the antibody or antigen-binding fragment thereof comprises: HCDR1, HCDR2 and HCDR3 of the VH as set forth in SEQ ID NO: 40; and LCDR1, LCDR2 and LCDR3 of the VL as set forth in SEQ ID NO: 41, 42, 43, 44, or 45.

In certain embodiments, the antibody or antigen-binding fragment thereof comprises: HCDR1, HCDR2 and HCDR3 of the VH as set forth in SEQ ID NO: 46, 47, 48, 49, or 50; and LCDR1, LCDR2 and LCDR3 of the VL as set forth in SEQ ID NO: 41.

In certain embodiments, the antibody or antigen-binding fragment thereof comprises: HCDR1, HCDR2 and HCDR3 of the VH as set forth in SEQ ID NO: 50; and LCDR1, LCDR2 and LCDR3 of the VL as set forth in SEQ ID NO: 42 or 45.

In certain embodiments, the antibody or antigen-binding fragment thereof comprises: HCDR1, HCDR2 and HCDR3 of the VH as set forth in SEQ ID NO: 51; and LCDR1, LCDR2 and LCDR3 of the VL as set forth in SEQ ID NO: 42 or 45.

In certain embodiments, the antibody or antigen-binding fragment thereof comprises:
HCDR1, HCDR2 and HCDR3 comprising or consisting of the amino acid sequences of SEQ ID NOs: 98, 101, and 108, respectively; and LCDR1, LCDR2 and LCDR3 comprising or consisting of the amino acid sequences of SEQ ID NOs: 109, 114, and 117, respectively; or,
HCDR1, HCDR2 and HCDR3 comprising or consisting of the amino acid sequences of SEQ ID NOs: 98, 101 and 108, respectively; and LCDR1, LCDR2 and LCDR3 comprising or consisting of the amino acid sequences of SEQ ID NOs: 109, 115 and 117, respectively; or,
HCDR1, HCDR2 and HCDR3 comprising or consisting of the amino acid sequences of SEQ ID NOs: 98, 101 and 108, respectively; and LCDR1, LCDR2 and LCDR3 comprising or consisting of the amino acid sequences of SEQ ID NOs: 110, 115 and 117, respectively; or,
HCDR1, HCDR2 and HCDR3 comprising or consisting of the amino acid sequences of SEQ ID NOs: 98, 101 and 108, respectively; and LCDR1, LCDR2 and LCDR3 comprising or consisting of the amino acid sequences of SEQ ID NOs: 111, 115 and 117, respectively; or,
HCDR1, HCDR2 and HCDR3 comprising or consisting of the amino acid sequences of SEQ ID NOs: 98, 101 and 108, respectively; and LCDR1, LCDR2 and LCDR3 comprising or consisting of the amino acid sequences of SEQ ID NOs: 112, 115 and 117, respectively; or,
HCDR1, HCDR2 and HCDR3 comprising or consisting of the amino acid sequences of SEQ ID NOs: 98, 102, and 108, respectively; and LCDR1, LCDR2 and LCDR3 comprising or consisting of the amino acid sequences of SEQ ID NOs: 109, 114 and 117, respectively; or,
HCDR1, HCDR2 and HCDR3 comprising or consisting of the amino acid sequences of SEQ ID NOs: 98, 103, and 108, respectively; and LCDR1, LCDR2 and LCDR3 comprising or consisting of the amino acid sequences of SEQ ID NOs: 109, 114 and 117, respectively; or,
HCDR1, HCDR2 and HCDR3 comprising or consisting of the amino acid sequences of SEQ ID NOs: 99, 102, and 108, respectively; and LCDR1, LCDR2 and LCDR3 comprising or consisting of the amino acid sequences of SEQ ID NOs: 109, 114 and 117, respectively; or,
HCDR1, HCDR2 and HCDR3 comprising or consisting of the amino acid sequences of SEQ ID NOs: 99, 104, and 108, respectively; and LCDR1, LCDR2 and LCDR3 comprising or consisting of the amino acid sequences of SEQ ID NOs: 109, 114 and 117, respectively; or,
HCDR1, HCDR2 and HCDR3 comprising or consisting of the amino acid sequences of SEQ ID NOs: 99, 105, and 108, respectively; and LCDR1, LCDR2 and LCDR3 comprising or consisting of the amino acid sequences of SEQ ID NOs: 109, 114 and 117, respectively; or,
HCDR1, HCDR2 and HCDR3 comprising or consisting of the amino acid sequences of SEQ ID NOs: 99, 105 and 108, respectively; and LCDR1, LCDR2 and LCDR3 comprising or consisting of the amino acid sequences of SEQ ID NOs: 109, 115 and 117, respectively; or,
HCDR1, HCDR2 and HCDR3 comprising or consisting of the amino acid sequences of SEQ ID NOs: 99, 105 and 108, respectively; and LCDR1, LCDR2 and LCDR3 comprising or consisting of the amino acid sequences of SEQ ID NOs: 112, 115 and 117, respectively; or,
HCDR1, HCDR2 and HCDR3 comprising or consisting of the amino acid sequences of SEQ ID NOs: 99, 106 and 108, respectively; and LCDR1, LCDR2 and LCDR3 comprising or consisting of the amino acid sequences of SEQ ID NOs: 109, 115 and 117, respectively; or,
HCDR1, HCDR2 and HCDR3 comprising or consisting of the amino acid sequences of SEQ ID NOs: 99, 106 and 108, respectively; and LCDR1, LCDR2 and LCDR3 comprising or consisting of the amino acid sequences of SEQ ID NOs: 112, 115 and 117, respectively.

In certain embodiments, the antibody or antigen-binding fragment thereof comprises a heavy chain variable region (VH) and a light chain variable region (VL), wherein: the VH comprises or consists of the sequence of SEQ ID NO: 10 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 11 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto. In certain embodiments, the VH comprises or consists of the sequence of SEQ ID NO: 10; and/or, the VL comprises or consists of the sequence of SEQ ID NO: 11.

In certain embodiments, the antibody or antigen-binding fragment thereof comprises a heavy chain variable region (VH) and a light chain variable region (VL), wherein: the VH comprises framework regions (FRs) derived from a human immunoglobulin heavy chain variable region sequence of GenBank: AXA20212; and/or, the VL comprises framework regions (FRs) derived from a human immunoglobulin light chain variable region sequence of GenBank: ABA70776.

In certain embodiments, the VH comprises or consists of the sequence of SEQ ID NO: 40, 46, 47, 48, 49, 50, or 51, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VL comprises or consists of the sequence of SEQ ID NO: 41, 42, 43, 44, or 45, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises a heavy chain variable region (VH) and a light chain variable region (VL), wherein:
the VH comprises or consists of the sequence of SEQ ID NO: 40, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 41, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 40, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 42, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 40, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 43, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 40, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 44, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 40, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 45, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 46, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 41 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 47, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 41 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 48, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 41 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 49, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 41 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 50, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 41 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 50, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 42, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 50, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 45, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 51, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 42, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto;
the VH comprises or consists of the sequence of SEQ ID NO: 51, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 45, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, (a) the VH comprises the sequence of SEQ ID NO: 50; and/or, the VL comprises the sequence of SEQ ID NO: 42; (b) the VH comprises the sequence of SEQ ID NO: 50; and/or, the VL comprises the sequence of SEQ ID NO: 45; (c) the VH comprises the sequence of SEQ ID NO: 51; and/or, the VL comprises the sequence of SEQ ID NO: 42; or (d) the VH comprises the sequence of SEQ ID NO: 51; and/or, the VL comprises the sequence of SEQ ID NO: 45.

### TIGIT-3

In certain embodiments, the antibody or antigen-binding fragment thereof of the disclosure is derived from murine antibody TIGIT-3 (having the VH of SEQ ID NO: 2 and the VL of SEQ ID NO: 3).

In certain embodiments, the antibody or antigen-binding fragment thereof comprises:
(i) HCDR1 comprising or consisting of the sequence of SEQ ID NO: 71;
(ii) HCDR2 comprising or consisting of the sequence of TIKSX₁GGSTNLX₂DSVKG (SEQ ID NO: 74), wherein X₁ is D or S, X₂ is P or A;
(iii) HCDR3 comprising or consisting of the sequence of SEQ ID NO: 75;
(iv) LCDR1 comprising or consisting of the sequence of X₃ASQDX₄KTAX₅A (SEQ ID NO: 78), wherein X₃ is Kor R, X₄ is V or I, X₅ is V or L;
(v) LCDR2 comprising or consisting of the sequence of WX₆STRX₇X₈ (SEQ ID NO: 82), wherein X₆ is S or A, X₇ is H or Q, X₈ is T or S; and
(vi) LCDR3 comprising or consisting of the sequence of SEQ ID NO: 83.

In certain embodiments, the HCDR2 comprises the sequence of SEQ ID NO: 72 or 73. In certain embodiments, the LCDR1 comprises the sequence of SEQ ID NO: 76 or 77. In certain embodiments, the LCDR2 comprises the sequence of SEQ ID NO: 79, 80, or 81.

In certain embodiments, the antibody or antigen-binding fragment thereof comprises: HCDR1, HCDR2 and HCDR3 of the VH as set forth in SEQ ID NO: 2, 22 or 23; and LCDR1, LCDR2 and LCDR3 of the VL as set forth in SEQ ID NO: 3, 24, 25, 26, 27, 28, or 29. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof comprises: HCDR1, HCDR2 and HCDR3 of the VH as set forth in SEQ ID NO: 2; and LCDR1, LCDR2 and LCDR3 of the VL as set forth in SEQ ID NO: 3.

In certain embodiments, the antibody or antigen-binding fragment thereof comprises: HCDR1, HCDR2 and HCDR3 of the VH as set forth in SEQ ID NO: 22 or 23; and LCDR1, LCDR2 and LCDR3 of the VL as set forth in SEQ ID NO: 3.

In certain embodiments, the antibody or antigen-binding fragment thereof comprises: HCDR1, HCDR2 and HCDR3 of the VH as set forth in SEQ ID NO: 22; and LCDR1, LCDR2 and LCDR3 of the VL as set forth in SEQ ID NO: 24, 25, 26, or 27.

In certain embodiments, the antibody or antigen-binding fragment thereof comprises: HCDR1, HCDR2 and HCDR3 of the VH as set forth in SEQ ID NO: 23; and LCDR1, LCDR2 and LCDR3 of the VL as set forth in SEQ ID NO: 28 or 29.

In certain embodiments, the antibody or antigen-binding fragment thereof comprises: HCDR1, HCDR2 and HCDR3 of the VH as set forth in SEQ ID NO: 23; and LCDR1, LCDR2 and LCDR3 of the VL as set forth in SEQ ID NO: 28.

In certain embodiments, the antibody or antigen-binding fragment thereof comprises:
HCDR1, HCDR2 and HCDR3 comprising or consisting of the amino acid sequences of SEQ ID NOs: 71, 72, and 75, respectively; and LCDR1, LCDR2 and LCDR3 comprising or consisting of the amino acid sequences of SEQ ID NOs: 76, 79, and 83, respectively; or,
HCDR1, HCDR2 and HCDR3 comprising or consisting of the amino acid sequences of SEQ ID NOs: 71, 73, and 75, respectively; and LCDR1, LCDR2 and LCDR3 comprising or consisting of the amino acid sequences of SEQ ID NOs: 76, 79, and 83, respectively; or,
HCDR1, HCDR2 and HCDR3 comprising or consisting of the amino acid sequences of SEQ ID NOs: 71, 72, and 75; and LCDR1, LCDR2 and LCDR3 comprising or consisting of the amino acid sequences of SEQ ID NOs: 76, 79, and 83, respectively; or,
HCDR1, HCDR2 and HCDR3 comprising or consisting of the amino acid sequences of SEQ ID NOs: 71, 72, and 75; and LCDR1, LCDR2 and LCDR3 comprising or consisting of the amino acid sequences of SEQ ID NOs: 77, 79, and 83, respectively; or,
HCDR1, HCDR2 and HCDR3 comprising or consisting of the amino acid sequences of SEQ ID NOs: 71, 72, and 75; and LCDR1, LCDR2 and LCDR3 comprising or consisting of the amino acid sequences of SEQ ID NOs: 76, 80, and 83, respectively; or,
HCDR1, HCDR2 and HCDR3 comprising or consisting of the amino acid sequences of SEQ ID NOs: 71, 72, and 75; and LCDR1, LCDR2 and LCDR3 comprising or consisting of the amino acid sequences of SEQ ID NOs: 76, 81, and 83, respectively; or,
HCDR1, HCDR2 and HCDR3 comprising or consisting of the amino acid sequences of SEQ ID NOs: 71, 73, and 75, respectively; and LCDR1, LCDR2 and LCDR3 comprising or consisting of the amino acid sequences of SEQ ID NOs: 77, 81, and 83, respectively.

In certain embodiments, the antibody or antigen-binding fragment thereof comprises a heavy chain variable region (VH) and a light chain variable region (VL), wherein: the VH comprises or consists of the sequence of SEQ ID NO: 2 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 3 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto. In certain embodiments, the VH comprises or consists of the sequence of SEQ ID NO: 2; and/or, the VL comprises or consists of the sequence of SEQ ID NO: 3.

In certain embodiments, the antibody or antigen-binding fragment thereof comprises a heavy chain variable region (VH) and a light chain variable region (VL), wherein: the VH comprises framework regions (FRs) derived from a human immunoglobulin heavy chain variable region sequence of GenBank: BAC02278; and/or, the VL comprises framework regions (FRs) derived from a human immunoglobulin light chain variable region sequence of GenBank: QTX15665.

In certain embodiments, the VH comprises or consists of the sequence of SEQ ID NO: 22 or 23, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VL comprises or consists of the sequence of SEQ ID NO: 24, 25, 26, 27, 28, or 29, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises a heavy chain variable region (VH) and a light chain variable region (VL), wherein:
the VH comprises or consists of the sequence of SEQ ID NO: 22, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 3 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 23, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 3 or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 22, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 24, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 22, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 25, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 22, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 26, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 22, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 27, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 23, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 28, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
the VH comprises or consists of the sequence of SEQ ID NO: 23, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, the VL comprises or consists of the sequence of SEQ ID NO: 29, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the VH comprises or consists of the sequence of SEQ ID NO: 23; and/or, the VL comprises or consists of the sequence of SEQ ID NO: 28.

### TIGIT-15, -18, -52, -63, -87, -94, -100

In certain embodiments, the antibody or antigen-binding fragment thereof of the disclosure is derived from murine antibody TIGIT-15, -18, -52, -63, -87, -94, or -100.

In certain embodiments, the antibody or antigen-binding fragment thereof of the disclosure comprises: HCDR1, HCDR2, HCDR3, LCDR1, LCDR2 and LCDR3 comprising or consisting of the amino acid sequences of SEQ ID NOs: 118, 119, 120, 121, 122, and 123, respectively.

In certain embodiments, the antibody or antigen-binding fragment thereof comprises: HCDR1, HCDR2 and HCDR3 of the VH as set forth in SEQ ID NO: 6; and LCDR1, LCDR2 and LCDR3 of the VL as set forth in SEQ ID NO: 7. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof of the disclosure comprises: a VH comprising or consisting of the sequence of SEQ ID NO: 6, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising or consisting of the sequence of SEQ ID NO: 7, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising or consisting of the amino acid sequences of SEQ ID NOs: 124, 125, 126, 127, 128, and 129, respectively.

In certain embodiments, the antibody or antigen-binding fragment thereof comprises: HCDR1, HCDR2 and HCDR3 of the VH as set forth in SEQ ID NO: 8; and LCDR1, LCDR2 and LCDR3 of the VL as set forth in SEQ ID NO: 9. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof of the disclosure comprises: a VH comprising or consisting of the sequence of SEQ ID NO: 8, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising or consisting of the sequence of SEQ ID NO: 9, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising or consisting of the amino acid sequences of SEQ ID NOs: 130, 131, 132, 133, 134, and 135, respectively.

In certain embodiments, the antibody or antigen-binding fragment thereof comprises: HCDR1, HCDR2 and HCDR3 of the VH as set forth in SEQ ID NO: 12; and LCDR1, LCDR2 and LCDR3 of the VL as set forth in SEQ ID NO: 13. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof of the disclosure comprises: a VH comprising or consisting of the sequence of SEQ ID NO: 12, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising or consisting of the sequence of SEQ ID NO: 13, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising or consisting of the amino acid sequences of SEQ ID NOs: 136, 137, 138, 139, 140, and 141, respectively.

In certain embodiments, the antibody or antigen-binding fragment thereof comprises: HCDR1, HCDR2 and HCDR3 of the VH as set forth in SEQ ID NO: 14; and LCDR1, LCDR2 and LCDR3 of the VL as set forth in SEQ ID NO: 15. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof of the disclosure comprises: a VH comprising or consisting of the sequence of SEQ ID NO: 14, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising or consisting of the sequence of SEQ ID NO: 15, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising or consisting of the amino acid sequences of SEQ ID NOs: 142, 143, 144, 145, 146 and 147, respectively.

In certain embodiments, the antibody or antigen-binding fragment thereof comprises: HCDR1, HCDR2 and HCDR3 of the VH as set forth in SEQ ID NO: 16; and LCDR1, LCDR2 and LCDR3 of the VL as set forth in SEQ ID NO: 17. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof of the disclosure comprises: a VH comprising or consisting of the sequence of SEQ ID NO: 16, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising or consisting of the sequence of SEQ ID NO: 17, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising or consisting of the amino acid sequences of SEQ ID NOs: 148, 149, 150, 151, 152 and 153, respectively.

In certain embodiments, the antibody or antigen-binding fragment thereof comprises: HCDR1, HCDR2 and HCDR3 of the VH as set forth in SEQ ID NO: 18; and LCDR1, LCDR2 and LCDR3 of the VL as set forth in SEQ ID NO: 19. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof of the disclosure comprises: a VH comprising or consisting of the sequence of SEQ ID NO: 18, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising or consisting of the sequence of SEQ ID NO: 19, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof of the disclosure comprises: HCDR1, HCDR2, HCDR3, LCDR1, LCDR2 and LCDR3 comprising or consisting of the amino acid sequences of SEQ ID NOs: 154, 155, 156, 157, 158 and 159, respectively.

In certain embodiments, the antibody or antigen-binding fragment thereof comprises: HCDR1, HCDR2 and HCDR3 of the VH as set forth in SEQ ID NO: 20; and LCDR1, LCDR2 and LCDR3 of the VL as set forth in SEQ ID NO: 21. In certain embodiments, the CDRs are defined according to Kabat, AbM, IMGT, or Chothia numbering system, or any combination thereof. In certain embodiments, the CDRs are defined according to Abm numbering system (for HCDR1) and Kabat numbering system (for HCDR2 and 3, LCDR1-3).

In certain embodiments, the antibody or antigen-binding fragment thereof of the disclosure comprises: a VH comprising or consisting of the sequence of SEQ ID NO: 20, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a VL comprising or consisting of the sequence of SEQ ID NO: 21, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

### Constant Region

The antibody or antigen-binding fragment thereof of the present disclosure may further comprise a constant region sequence derived from a mammalian (e.g., murine or human) immunoglobulin.

In certain embodiments, the antibody or antigen-binding fragment thereof of the present disclosure comprise a constant region sequence derived from a human immunoglobulin.

Any immunoglobulin constant region can be used in the antibodies disclosed herein. In certain embodiments, the heavy chain constant region can be a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. Light chain constant region can be lambda or kappa. In certain embodiments, the heavy chain constant region is an IgG heavy chain constant region, such as an IgG1, IgG2, IgG3 or IgG4 heavy chain constant region. In certain embodiments, the light chain constant region is kappa.

The choice of constant region depends, in part, whether antibody-dependent complement and/or cellular mediated cytotoxicity is desired. In certain embodiments, the heavy chain constant region with less or reduced effector function is preferred. In certain embodiments, the heavy chain constant region with effector function or enhanced effector function is preferred. In certain embodiments, the heavy chain constant region with ADCC or enhanced ADCC is preferred.

In certain embodiments, the heavy chain of the antibody or antigen-binding fragment thereof of the present disclosure comprises a heavy chain constant region (CH) comprising an amino acid sequence derived from a human immunoglobulin heavy chain constant region.

In certain embodiments, the antibody or antigen-binding fragment thereof of the present disclosure comprises a heavy chain constant region that is a wild-type heavy chain constant region.

In certain embodiments, the antibody or antigen-binding fragment thereof of the present disclosure comprises a heavy chain constant region that is a variant of a wild-type heavy chain constant region. Mutation(s) (e.g., one or more amino acid substitutions) can be introduced into the Fc region of constant region to alter (increase, reduce or eliminate) the effector function(s) of the antibody, such as ADCC, CDC or ADCP compared with the same antibody without the mutation(s).

In certain embodiments, the heavy chain constant region comprises or consists of the amino acid sequence of SEQ ID NO: 69.

In certain embodiments, the light chain of the antibody or antigen-binding fragment thereof of the present disclosure comprises a light chain constant region (CL) comprising an amino acid sequence derived from a human immunoglobulin light chain constant region.

In certain embodiments, the light chain constant region comprises or consists of the amino acid sequence of SEQ ID NO: 70.

### Full-Length Antibody

The antibody of the disclosure may be an antibody comprising two heavy chains and two light chains, having a conventional “Y” type structure.

In certain embodiments, the antibody or antigen-binding fragment thereof is derived from TIGIT-6, comprising: a heavy chain comprising or consisting of the sequence of SEQ ID NO: 56, 58, 59, 60, or 61, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 57 or 62, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises:
a heavy chain comprising or consisting of the sequence of SEQ ID NO: 56, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 57, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
a heavy chain comprising or consisting of the sequence of SEQ ID NO: 58, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 62, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
a heavy chain comprising or consisting of the sequence of SEQ ID NO: 59, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 62, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
a heavy chain comprising or consisting of the sequence of SEQ ID NO: 60, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 62, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
a heavy chain comprising or consisting of the sequence of SEQ ID NO: 61, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 62, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof is derived from TIGIT-19, comprising: a heavy chain comprising or consisting of the sequence of SEQ ID NO: 63, 65, or 66, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 64, 67, or 68, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises:
a heavy chain comprising or consisting of the sequence of SEQ ID NO: 63, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 64, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
a heavy chain comprising or consisting of the sequence of SEQ ID NO: 65, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 67, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
a heavy chain comprising or consisting of the sequence of SEQ ID NO: 65, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 68, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
a heavy chain comprising or consisting of the sequence of SEQ ID NO: 66, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 67, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
a heavy chain comprising or consisting of the sequence of SEQ ID NO: 66, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 68, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof is derived from TIGIT-3, comprising: a heavy chain comprising or consisting of the sequence of SEQ ID NO: 52 or 54, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 53 or 55, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

In certain embodiments, the antibody or antigen-binding fragment thereof comprises:
a heavy chain comprising or consisting of the sequence of SEQ ID NO: 52, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 53, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; or,
a heavy chain comprising or consisting of the sequence of SEQ ID NO: 54, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto; and/or, a light chain comprising or consisting of the sequence of SEQ ID NO: 55, or an amino acid sequence having at least 80% (e.g., at least 85%, at least 90%, at least 91%, at least 92%, at least 93%, at least 94%, at least 95%, at least 96%, at least 97%, at least 98%, at least 99%, or 100%) sequence identity thereto.

### Antigen-Binding Fragment

The antibody of the present disclosure may also be a Fab fragment, Fab′, F(ab)₂, Fv, scFv, or any other type of fragment of an antibody having a conventional “Y” type structure, which substantially retains the ability to specifically bind to TIGIT (e.g., human TIGIT) and block binding of TIGIT (e.g., human TIGIT) to PVR/CD155.

In certain embodiments, the antibody or antigen-binding fragment thereof of the disclosure is selected from the group consisting of scFv, Fab, Fab′, (Fab′)₂, Fv fragments, diabodies, bispecific antibodies, multispecific antibodies, chimeric antibodies, or humanized antibodies.

The antibody or antigen-binding fragment thereof of the present disclosure has one or more of the following properties: (1) has high specificity and high affinity to TIGIT (e.g., human TIGIT and/or cynomolgus TIGIT), for example, with a KD of about 1×10⁻⁹ M or less, preferably about 1×10⁻¹⁰ M or less, more preferably about 1×10⁻¹¹ M or less, as measured by SPR: (2) blocks binding of TIGIT (e.g., human TIGIT) to PVR/CD155, for example, with an EC50 of about 10 nM or less, about 9 nM or less, about 5 nM or less, as measured by TIGIT/CD155 blockade reporter assay as defined in EXAMPLE 3; (3) increases T cell activation, for example, as measured by promoted secretion of IFN-γ and/or IL-2 when contacting T cells with the antibody, as compared to T cell activation in the absence of the anti-TIGIT antibody; (4) reduces or depletes Tregs, for example, as measured by a flow cytometry-based ADCC assay as described in EXAMPLE 5 when contacting effector cells with PBMC target cells in the presence of the anti-TIGIT antibody; (5) treat tumor, for example, as measured by tumor growth inhibition in CT26 tumor model; (6) treat an infectious disease.

Also provided are “conservative sequence modifications” to the antibody sequence provided herein, i.e., nucleotide and amino acid sequence modifications that do not abrogate the binding of the antibody encoded by the nucleotide sequence or containing the amino acid sequence, to the antigen. For example, modifications can be introduced by standard techniques known in the art, such as site-directed mutagenesis and PCR-mediated mutagenesis. The resulting modified antibodies can be screened for its binding activity.

Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in the antibodies disclosed herein could be preferably replaced with another amino acid residue from the same side chain family. Methods of identifying nucleotide and amino acid conservative substitutions that do not eliminate antigen binding are well-known in the art. See, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

### Derivatized Antibodies

The antibody or an antigen-binding fragment thereof of the disclosure can be derivatized, for example, linked to another molecule (e.g., another polypeptide or protein). In general, the derivatization (such as labeling) of an antibody or an antigen-binding fragment thereof would not affect its binding to TIGIT adversely. Therefore, the antibody or an antigen-binding fragment thereof of the disclosure is also intended to include such derivatized forms. For example, the antibody or an antigen-binding fragment thereof of the disclosure can be functionally linked (by chemical coupling, genetic fusion, non-covalent linkage or other means) to one or more other molecular groups, such as another antibody (e.g. forming a bispecific antibody), a detection agent, a medicinal agent, and/or a protein or polypeptide capable of mediating associate of the antibody or an antigen binding fragment thereof with another molecule (such as an avidin or a polyhistidine-tag).

### Labeled Antibodies

In certain embodiments, the antibody or antigen-binding fragment thereof of the present disclosure is labeled, such as a detectable label. The detectable label may be any substance that can be detected directly or indirectly, e.g., through an enzymatic reaction or molecular interaction. In certain embodiments, the label can be detected by fluorescent, spectroscopic, photochemical, biochemical, immunological, electrical, optical, or chemical means. In certain embodiments, the detectable label can be suitable for immunological detection (e.g., enzyme-linked immunoassay; radioimmunoassay, fluorescent immunoassay, chemiluminescence immunoassay, etc.). In certain embodiments, the label is selected from an enzyme, a radionuclide, a fluorescent dye, a luminescent substance (e.g., a chemiluminescent substance), or a biotin. In certain embodiments, the label can be linked to the antibody or antigen-binding fragment thereof of the disclosure via linkers of different lengths to reduce potential steric hindrance.

In certain embodiments, the labeled antibody or antigen-binding fragment thereof as described herein can be useful for detecting the presence of TIGIT in a biological sample. The term “detecting” as used herein encompasses quantitative or qualitative detection. In certain embodiments, the biological sample is blood, serum or other liquid samples of biological origin. In certain embodiments, the biological sample comprises a cell or tissue.

In certain embodiments, there is provided a method of detecting TIGIT in a cell, comprising contacting the cell with the labeled antibody or antigen-binding fragment thereof as described herein. In certain embodiments, a method of detecting the presence of TIGIT in a biological sample is provided. In certain embodiments, the method comprises detecting the presence of TIGIT protein in a biological sample. In certain embodiments, the TIGIT is human TIGIT. In certain embodiments, the method comprises contacting the biological sample with the labeled antibody or antigen-binding fragment thereof as described herein under conditions permissive for binding of the antibody or antigen-binding fragment thereof to TIGIT, and detecting signal from the label. Such method may be an in vitro or in vivo method. In certain embodiments, there is provided a method of diagnosing a disease associated with TIGIT expression (e.g., tumor or infectious disease) in an individual, comprising administering to the individual the labeled antibody or antigen-binding fragment thereof as described herein, and detecting the label in the individual. In certain embodiments, the labeled antibody or antigen-binding fragment thereof as described herein is used to select subjects eligible for therapy with any of anti-TIGIT therapeutic agents (e.g., the antibody or antigen-binding fragment thereof, the immunoconjugate, or the bispecific or multispecific antibody as described herein), wherein TIGIT is a biomarker for selection of patients.

### Immunoconjugates

In another aspect, there is provided an immunoconjugate comprising the antibody or antigen-binding fragment thereof as described herein and an effector molecule. Exemplary effector molecules include, but are not limited to, a drug, a toxin, a radioisotope, a protein, a peptide, and a nucleic acid. In certain embodiments, the effector molecule is a therapeutic agent.

In certain embodiments, the immunoconjugate of the disclosure comprises the antibody or antigen-binding fragment thereof as described herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In certain embodiments, the immunoconjugate of the disclosure is an antibody-drug conjugate (ADC) in which the antibody or antigen-binding fragment thereof as described herein is conjugated to one or more drugs, including but not limited to a maytansinoid (see U.S. Pat. Nos. 5,208,020, 5,416,064 and European Patent EP0425235B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53: 3336-3342 (1993); and Lode et al., Cancer Res. 58: 2925-2928 (1998)); an anthracycline such as daunomycin or doxorubicin (see Kratz et al., Current Med. Chem. 13: 477-523 (2006); Jeffrey et al., Bioorganic &Med. Chem. Letters 16: 358-362 (2006); Torgov et al., Bioconj. Chem. 16: 717-721 (2005); Nagy et al., Proc. Natl. Acad. Sci. USA 97: 829-834 (2000); Dubowchik et al., Bioorg. &Med. Chem. Letters 12: 1529-1532 (2002); King et al., J. Med. Chem. 45: 4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In certain embodiments, the immunoconjugate of the disclosure comprises the antibody or antigen-binding fragment thereof as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In certain embodiments, the immunoconjugate of the disclosure comprises the antibody or antigen-binding fragment thereof as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, “MRI”), such as iodine-123, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2, 6-diisocyanate), and bis-active fluorine compounds (such as 1, 5-difluoro-2, 4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52: 127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone) benzoate) which are commercially available (e.g., from Pierce Biotechnology, Inc., Rockford, IL., U.S.A).

### Bispecific Antibodies

The antibody or antigen-binding fragment thereof of the disclosure can be used for forming bispecific or multispecific antibodies. The antibody or antigen-binding fragment thereof of the disclosure may be a part of a bispecific or multispecific antibody that includes a second functional module (e.g., a second antibody) having a binding specificity different from that of the antibody or antigen-binding fragment thereof of the disclosure, so that it is capable of binding to at least two different binding sites and/or target molecules. For example, the antibody or antigen-binding fragment thereof of the disclosure can be linked to a second antibody or antigen-binding fragment thereof that specifically binds to any protein that can be used as a potential target for combination therapy. To generate the bispecific or multispecific antibody, the antibody or antigen-binding fragment thereof of the disclosure can be linked (e.g., by chemical coupling, gene fusion, non-covalent association, or other means) to one or more other binding molecules (e.g., additional antibodies, antibody fragments, peptides, or binding mimics).

Accordingly, in another aspect, the disclosure provides a bispecific or multispecific antibody, comprising the antibody or antigen-binding fragment thereof of the disclosure.

In certain embodiments, the bispecific or multispecific antibody specifically binds to TIGIT (e.g., human TIGIT) and a second target. In certain embodiments, the bispecific or multispecific antibody comprises a first antigen binding domain from the antibody or an antigen binding fragment thereof of the disclosure and a second antigen binding domain from an antibody against a second target.

In certain embodiments, the second target is a different immunomodulatory receptor.

### Production of Antibodies

Antibodies disclosed herein can be obtained by genetic engineering recombination techniques. For example, DNA molecules of genes encoding the heavy and light chains of the antibodies of the disclosure can be obtained by chemical synthesis or PCR amplification. The resulting DNA molecule is inserted into an expression vector and then transfected into a host cell, such as HEK293 cell, or other cells that do not produce an immunoglobulin. Then, the transfected host cells are cultured under specific conditions and express the antibody of the present disclosure.

Antigen-binding fragments disclosed herein can be obtained by hydrolysis of an intact antibody molecule. Alternatively, these antigen-binding fragments can be produced directly from recombinant host cells (reviewed in Hudson, curr. Opin. Immunol. 11: 548-557 (1999); Little et al., Immunol. Today, 21: 364-370 (2000)). For example, the Fab′ fragment can be obtained directly from recombinant host cells; and the Fab′ fragments can be chemically coupled to form an F(ab′)₂ fragment (Carter et al., Bio/Technology, 10: 163-167 (1992)). Furthermore, the Fv, Fab or F(ab′)₂ fragments can also be isolated directly from a culture of recombinant host cells. Other techniques for preparing these antigen-binding fragments are well known to those of ordinary skill in the art.

In another aspect, the disclosure provides an isolated nucleic acid molecule, comprising a nucleotide sequence encoding the antibody or an antigen binding fragment thereof of the disclosure, or its heavy chain variable region and/or light chain variable region, or the bispecific or multispecific antibody of the disclosure.

In certain embodiments, the isolated nucleic acid molecule comprises a first nucleotide sequence and a second nucleotide sequence encoding the heavy chain variable region and the light chain variable region of the antibody or antigen-binding fragment thereof of the present disclosure, respectively.

In certain embodiments, the isolated nucleic acid molecule comprises a first nucleotide sequence and a second nucleotide sequence encoding the heavy chain and the light chain of the antibody or antigen-binding fragment thereof of the present disclosure, respectively.

In certain embodiments, the isolated nucleic acid molecule comprises different nucleotide sequences encoding different polypeptide chains of the bispecific or multispecific antibody respectively.

In another aspect, the disclosure provides a vector (e.g., a cloning vector or an expression vector), comprising the isolated nucleic acid molecule of the disclosure.

In certain embodiments, the vector of the disclosure is, for example, a plasmid, a cosmid, a phage, etc. In certain embodiments, the vector can express the antibody or an antigen-binding fragment thereof of the disclosure in a subject (for example, mammal, such as human).

In certain embodiments, the vector comprises a first nucleotide sequence and a second nucleotide sequence encoding the heavy chain variable region and the light chain variable region of the antibody or antigen-binding fragment thereof of the present disclosure, respectively. The first and second nucleotide sequences can be located on same or different vectors.

In certain embodiments, the vector comprises a first nucleotide sequence and a second nucleotide sequence encoding the heavy chain and the light chain of the antibody or antigen-binding fragment thereof of the present disclosure, respectively. The first and second nucleotide sequences can be located on same or different vectors.

In certain embodiments, the vector comprises different nucleotide sequences encoding different polypeptide chains of the bispecific or multispecific antibody respectively. The different nucleotide sequences can be located on same or different vectors.

In another aspect, the disclosure provides a host cell, comprising or is transformed with the isolated nucleic acid molecule of the disclosure or the vector of the disclosure. Such host cells include, but are not limited to, prokaryotic cell such as E. coli cell, and eukaryotic cell such as yeast cell, insect cell, plant cell and animal cell (e.g., mammalian cell, such as mouse cell and human cell).

In another aspect, provided is a method for producing the antibody or an antigen-binding fragment thereof of the disclosure, or the bispecific or multispecific antibody of the disclosure, comprising, culturing a host cell comprising the isolated nucleic acid molecule of the disclosure or the vector of the disclosure, or the host cell of the disclosure under a condition allowing expression of the antibody or an antigen-binding fragment thereof or the bispecific or multispecific antibody, and recovering the antibody or an antigen-binding fragment thereof or the bispecific or multispecific antibody from a culture of the cultured host cell.

### Pharmaceutical Composition

In one aspect, the disclosure provides a pharmaceutical composition, comprising the antibody or antigen-binding fragment thereof of the disclosure, or the immunoconjugate of the disclosure, the bispecific or multispecific antibody of the disclosure, or the isolated nucleic acid molecule, vector, or host cell of the disclosure, and a pharmaceutically acceptable carrier and/or excipient.

In certain embodiments, the pharmaceutical composition comprises the antibody or antigen-binding fragment thereof of the disclosure. In certain embodiments, the pharmaceutical composition comprises an effective amount of the antibody or antigen-binding fragment thereof of the disclosure. In certain embodiments, the antibody or antigen-binding fragment thereof is the only active ingredient included in the pharmaceutical composition.

In certain embodiments, the pharmaceutical composition comprises the immunoconjugate of the disclosure. In certain embodiments, the pharmaceutical composition comprises an effective amount of the immunoconjugate of the disclosure. In certain embodiments, the immunoconjugate is the only active ingredient included in the pharmaceutical composition.

In certain embodiments, the pharmaceutical composition comprises the bispecific or multispecific antibody of the disclosure. In certain embodiments, the pharmaceutical composition comprises an effective amount of the bispecific or multispecific antibody of the disclosure. In certain embodiments, the bispecific or multispecific antibody is the only active ingredient included in the pharmaceutical composition.

In certain embodiments, the pharmaceutical composition of the disclosure may further comprise an additional therapeutic agent. In certain embodiments, the additional therapeutic agent is an anti-tumor agent. In certain embodiments, the additional therapeutic agent is an additional immune checkpoint inhibitor, such as anti-PD-1 antibody, anti-PD-L1 antibody, anti-TIM-3 antibody, anti-LAG-3 antibody, or anti-CTLA-4 antibody. In certain embodiments, the additional therapeutic agent is a cytotoxic agent, such as an alkylating agent, an anti-mitotic agent, an antitumor antibiotic, an antimetabolite, a topoisomerase inhibitor, a tyrosine kinase inhibitor, or a radionuclide.

In certain embodiments, the antibody or antigen-binding fragment thereof of the disclosure or the immunoconjugate of the disclosure or the bispecific or multispecific antibody of the disclosure, and the additional therapeutic agent may be provided as separate components or as components of a single composition. The antibody or the antigen-binding fragment thereof of the disclosure or the immunoconjugate of the disclosure or the bispecific or multispecific antibody of the disclosure may be used in combination with the other agents simultaneously, separately, or successively.

The pharmaceutical composition can be provided in unit dosage form (i.e., the dosage for a single administration).

The pharmaceutical composition can be formulated using one or more pharmaceutically acceptable carriers and/or excipients. The formulation depends on the route of administration chosen. For parenteral administration, the pharmaceutical composition is preferably sterile and substantially isotonic and manufactured under GMP conditions. By way of example, the antibodies disclosed herein can be formulated in aqueous solutions for injection, preferably in physiologically compatible buffers, such as water for injection (WFI), bacteriostatic water for injection (BWFI), sodium chloride solution (e.g., 0.9% (w/v) NaCl), glucose solution (e.g., 5% glucose), surfactant-containing solution (e.g., 0.01% polysorbate 20), pH buffered solution (e.g., phosphate buffered solution), Ringer's solution. The solution can contain formulatory agents such as suspending, stabilizing and/or dispersing agents. Alternatively, antibodies can be in lyophilized form for constitution with a suitable vehicle, e.g., sterile pyrogen-free water, before use.

Pharmaceutical compositions described herein can be useful in increasing immune cell activation, enhancing an immune response, reducing or eliminating TIGIT-mediated immune suppression, reducing or depleting regulatory T cells, and/or treating a condition, such as tumor or an infectious disease.

### Uses and Methods

The antibody or antigen-binding fragment thereof of the present disclosure can specifically bind to TIGIT (e.g., human TIGIT) and block binding to PVR/CD155, thereby reducing or eliminating TIGIT-mediated immune suppression. Accordingly, the antibodies described herein may be used in a treatment in a wide variety of therapeutic applications, including, treating diseases associated with immunosuppression, for example, inhibiting tumor growth and treating infections.

In one aspect, the disclosure provides a method of increasing immune cell (e.g., T cell and/or NK cell) activation in response to an antigen in a subject (e.g., human), comprising administering to a subject in need thereof a therapeutically effective amount of the antibody or antigen-binding fragment thereof, immunoconjugate, bispecific or multispecific antibody, isolated nucleic acid molecule, vector, host cell, or pharmaceutical composition disclosed herein. In certain embodiments, the subject has a tumor and the antigen is a tumor antigen. In another embodiment, the subject has an infectious disease, for example, caused by a pathogen (e.g., a virus, bacteria, fungi, or protozoan), and the antigen is an infectious disease antigen, e.g., on surface of the pathogen.

In one aspect, the disclosure provides a method of enhancing an immune response in a subject (e.g., human), comprising administering to a subject in need thereof a therapeutically effective amount of the antibody or antigen-binding fragment thereof, immunoconjugate, bispecific or multispecific antibody, isolated nucleic acid molecule, vector, host cell, or pharmaceutical composition disclosed herein. In certain embodiments, the subject has a tumor and an immune response against the tumor is enhanced. In another embodiment, the subject has an infectious disease, for example, caused by a pathogen (e.g., a virus, bacteria, fungi, or protozoan), and an immune response against the pathogen is enhanced. In certain embodiments, the immune response is antigen-specific T cell response.

In one aspect, the disclosure provides a method of reducing or eliminating TIGIT-mediated immune suppression in a subject (e.g., human), comprising administering to a subject in need thereof a therapeutically effective amount of the antibody or antigen-binding fragment thereof, immunoconjugate, bispecific or multispecific antibody, isolated nucleic acid molecule, vector, host cell, or pharmaceutical composition disclosed herein. In certain embodiments, the subject has a tumor, for example, in which TIGIT (e.g., TIGIT-mediated signaling or TIGIT-expressing regulatory T cells) suppresses anti-tumor immune response. In another embodiment, the subject has an infectious disease, for example, caused by a pathogen (e.g., a virus, bacteria, fungi, or protozoan), and in which TIGIT (e.g., TIGIT-mediated signaling or TIGIT-expressing regulatory T cells) suppresses anti-pathogen immune response.

In one aspect, the disclosure provides a method of reducing or depleting regulatory T cells in a tumor of a subject (e.g., human), comprising administering to a subject in need thereof a therapeutically effective amount of the antibody or antigen-binding fragment thereof, immunoconjugate, bispecific or multispecific antibody, isolated nucleic acid molecule, vector, host cell, or pharmaceutical composition disclosed herein. In certain embodiments, the subject has a tumor, for example, in which TIGIT (e.g., TIGIT-mediated signaling or TIGIT-expressing regulatory T cells) suppresses anti-tumor immune response.

In one aspect, the disclosure provides a method of treating tumor in a subject (e.g., human), comprising administering to a subject in need thereof a therapeutically effective amount of the antibody or antigen-binding fragment thereof, immunoconjugate, bispecific or multispecific antibody, isolated nucleic acid molecule, vector, host cell, or pharmaceutical composition disclosed herein. In certain embodiments, the subject has a tumor, for example, in which TIGIT (e.g., TIGIT-mediated signaling or TIGIT-expressing regulatory T cells) suppresses anti-tumor immune response.

In one aspect, the disclosure provides a method of treating an infectious disease in a subject (e.g., human), comprising administering to a subject in need thereof a therapeutically effective amount of the antibody or antigen-binding fragment thereof, immunoconjugate, bispecific or multispecific antibody, isolated nucleic acid molecule, vector, host cell, or pharmaceutical composition disclosed herein. In certain embodiments, the subject has an infectious disease, for example, caused by a pathogen (e.g., a virus, bacteria, fungi, or protozoan), and in which TIGIT (e.g., TIGIT-mediated signaling or TIGIT-expressing regulatory T cells) suppresses anti-pathogen immune response.

In another aspect, the disclosure provides use of the antibody or antigen-binding fragment thereof, immunoconjugate, bispecific or multispecific antibody, isolated nucleic acid molecule, vector, host cell, or pharmaceutical composition disclosed herein in the manufacture of a medicament for use in (a) increasing immune cell activation in response to an antigen in a subject; (b) enhancing an immune response in a subject; (c) reducing or eliminating TIGIT-mediated immune suppression in a subject; (d) reducing or depleting regulatory T cells in a tumor of a subject; (e) treating tumor in a subject; and/or (f) treating an infectious disease in a subject.

In another aspect, the disclosure relates to the antibody or antigen-binding fragment thereof, immunoconjugate, bispecific or multispecific antibody, isolated nucleic acid molecule, vector, host cell, or pharmaceutical composition disclosed herein for use in (a) increasing immune cell activation in response to an antigen in a subject; (b) enhancing an immune response in a subject; (c) reducing or eliminating TIGIT-mediated immune suppression in a subject; (d) reducing or depleting regulatory T cells in a tumor of a subject; (e) treating tumor in a subject; and/or (f) treating an infectious disease in a subject.

In certain embodiments, the tumor involved in the methods or uses of modulating immune function and methods or uses of treatment described herein include, without limitation, solid tumors and hematological malignancies. The tumor also can be a metastatic cancer, refractory cancer, or recurrent cancer. Such tumors may or may not express TIGIT or CD155. Antibodies to TIGIT are effective against cancers not expressing TIGIT because inhibition of TIGIT interaction with CD155 stimulates an immune response against such cancers. Examples of solid tumors include, without limitation, ovarian cancer, endometrial cancer, breast cancer, lung cancer (small cell or non-small cell), colon cancer, prostate cancer, cervical cancer, pancreatic cancer, gastric cancer, esophageal cancer, hepatocellular carcinoma (liver cancer), renal cell carcinoma (kidney cancer), head-and-neck tumors, mesothelioma, melanoma, sarcomas, and brain tumors (e.g., gliomas, such as glioblastomas). Examples of hematological malignancies include leukemias, lymphomas and myelomas, including acute myeloid leukemia, adult T-cell leukemia, T-cell large granula lymphocyte leukemia, acute lymphoblastic leukemia, chronic lymphocytic leukemia, chronic myelogenous leukemia, acute monocytic leukemia, Hodgkin's and Non-Hodgkin's lymphoma and multiple myeloma. In certain embodiments, the tumor involves TIGIT-mediated suppression of anti-tumor immune response, for example, via TIGIT-mediated signaling or TIGIT-expressing regulatory T cells. In certain embodiments, the tumor comprises one or more of the following: (a) high levels of infiltrating TIGIT-positive T cells and/or NK cells; (b) elevated expression of PVR and/or Nectin-2 on tumor cells or tumor infiltrating myeloid cells.

In certain embodiments, the infectious disease involved in the methods or uses of modulating immune function and methods or uses of treatment described herein include, without limitation, infections caused by any pathogen, such as a virus, bacteria, fungi, or protozoan. In certain embodiments, the infectious disease involves TIGIT-mediated suppression of anti-pathogen immune response, for example, via TIGIT-mediated signaling or TIGIT-expressing regulatory T cells.

In certain embodiments, in any of the methods or uses of modulating immune function and methods or uses of treatment described herein, the antibody, immunoconjugate or bispecific or multispecific antibody of the disclosure can be used alone. Alternatively, the antibody, immunoconjugate or bispecific or multispecific antibody of the disclosure can be used in combination with additional therapeutic agent. In certain embodiments, the additional therapeutic agent is an anti-tumor agent. In certain embodiments, the additional therapeutic agent is an additional immune checkpoint inhibitor, such as anti-PD-1 antibody, anti-PD-L1 antibody, anti-TIM-3 antibody, anti-LAG-3 antibody, or anti-CTLA-4 antibody. In certain embodiments, the additional therapeutic agent is a cytotoxic agent, such as an alkylating agent, an anti-mitotic agent, an antitumor antibiotic, an antimetabolite, a topoisomerase inhibitor, a tyrosine kinase inhibitor, or a radionuclide. In certain embodiments, the antibody, immunoconjugate or bispecific or multispecific antibody of the disclosure is used in combination with an additional therapy (e.g. standard cancer treatment, such as surgery, chemotherapy, radiation therapy, targeted therapy, immunotherapy, hormone therapy, gene therapy or palliative care).

In certain embodiments, in any of the methods or uses of modulating immune function and methods or uses of treatment described herein, the antibody or antigen-binding fragment thereof, the immunoconjugate, the bispecific or multispecific antibody; or pharmaceutical composition of the present disclosure can be formulated into any dosage form known in the medical field, for example, tablets, pills, suspensions, emulsions, solutions, gels, capsules, powders, granules, elixirs, lozenges, suppositories, injections (including injection liquids, sterile powders for injection and concentrated solutions for injection), inhalants, sprays, etc. The preferred dosage form depends on the intended route of administration and therapeutic use. One preferred dosage form is an injection. Such injection may be a sterile injectable solution. Alternatively, the sterile injectable solution can be prepared as a sterile lyophilized powder (e.g., by vacuum drying or freeze drying) for the convenience of storage and use.

In certain embodiments, in any of the methods or uses of modulating immune function and methods or uses of treatment described herein, the antibody or antigen-binding fragment thereof, the immunoconjugate, the bispecific or multispecific antibody, or pharmaceutical composition of the present disclosure can be administrated by any suitable method known in the art, including, but not limited to, oral, buccal, sublingual, eyeball, topical, parenteral, rectal, intrathecal, intracytoplasmic, groin, intravesical, local (e.g., powder, ointment or drops), or nasal route. However, for many therapeutic uses, the preferred route/mode of administration is parenteral administration (e.g., intravenous injection or bolus, subcutaneous injection, intraperitoneal injection, intramuscular injection). The skilled person will understand that the route and/or mode of administration will vary depending on the intended purpose. In a preferred embodiment, the antibodies disclosed herein are given by intravenous injection or bolus.

In certain embodiments, in any of the methods or uses of modulating immune function and methods or uses of treatment described herein, the subject is a human. In certain embodiments, the subject has a tumor. In certain embodiments, the tumor comprises one or more of the following: (a) high levels of infiltrating TIGIT-positive T cells and/or NK cells; (b) elevated expression of PVR and/or Nectin-2 on tumor cells or tumor infiltrating myeloid cells.

In certain embodiments, in any of the methods or uses of modulating immune function and methods or uses of treatment described herein, the antibody has effector function (e.g., ADCC) or enhanced effector function (e.g., ADCC).

### EXAMPLES

The following examples discuss the production, characterization, and humanization of monoclonal antibodies against human TIGIT and also provide exemplary methods by which the activities of binding, blocking, ADCC and tumor growth inhibition by which the antibodies described in this application can be determined.

The examples provided below are for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

### Example 1: Immunization, Single Cell Sorting and Sequencing

Recombinant human TIGIT (SEQ ID NO: 1, ACROBiosystems, cat. #: TIT-H5254) was used to immunize female Balb/c mice intraperitoneally each with 50 μg (for primary immunization) or 25 μg (for three boosts) of the protein in Freud complete adjuvant (Sigma-Aldrich) every 14 days over a period of 56 days. The spleens and lymph nodes were harvested on day 60. Single cell sorting and sequencing of TIGIT-specific antibodies from mouse memory B cells was carried out as previously described (von Boehmer L., Liu C., Ackerman S., Gitlin A. D., Wang Q., Gazumyan A., Nussenzweig M. C. Sequencing and cloning of antigen-specific antibodies from mouse memory B cells. Nat. Protoc. 2016; 11: 1908-1923). Briefly, single cell suspension of the spleen and lymph node was generated, and then total B cells were magnetically isolated and stained with fluorescently labeled antibodies. The fluorescently labeled antigen-specific mouse memory B cells were sorted by FACS into lysis buffer in a 96-well plate. For each cell, cDNA was generated by two-step reverse transcription (RT) using random primers. The variable heavy- and light-chain domains were then amplified by nested PCR. The first PCR was performed with a primer mix that anneals to the V (D) J leader sequences and an immunoglobulin constant region reverse primer. The second PCR was performed with primers annealing to the 5′ end of the variable (V) genes and an immunoglobulin nested constant region reverse primer. The PCR products were then purified and sequenced.

### Example 2: Production of Anti-TIGIT Chimeric Antibodies

The sequences generated in EXAMPLE 1 were analyzed and non-productive and redundant sequences were removed. Paired VH and VL sequences were used to construct full-length chimeric IgGs, that is, the VH and VL gene fragments were cloned into mammalian expression vectors to be in-frame with human IgG1 heavy chain (SEQ ID NO: 69) and kappa light chain constant regions (SEQ ID NO: 70), respectively. The resulting heavy and light chain plasmids were used to transfect HEK293 cells. Two days after transfection, supernatants were harvested. ELISA was carried out to detect the existence of anti-TIGIT antibody in the supernatant. Ten clones listed in Table 1 were confirmed to bind human TIGIT.

TABLE 1
variable domain sequence IDs of selected chimeric antibodies

SEQ ID

| mAb | VH | VL |
|---|---|---|
| TIGIT-3 | 2 | 3 |
| TIGIT-6 | 4 | 5 |
| TIGIT-15 | 6 | 7 |
| TIGIT-18 | 8 | 9 |
| TIGIT-19 | 10 | 11 |
| TIGIT-52 | 12 | 13 |
| TIGIT-63 | 14 | 15 |
| TIGIT-87 | 16 | 17 |
| TIGIT-94 | 18 | 19 |
| TIGIT-100 | 20 | 21 |

The plasmids of confirmed binders were prepared and used to transfect HEK293 cells. Supernatants were collected 4 days after transfection. Chimeric antibodies were purified by Protein A affinity chromatography and size exclusion chromatography. As positive controls, two anti-TIGIT antibodies 4.1D3 and Hu1217-2-2 were also produced in house using sequences in published patents (US20210032328A1 and WO2019129261A1, respectively).

### Example 3: In Vitro Characterization of Anti-TIGIT Chimeric Antibodies

### Binding of TIGIT Over-Expressing CHO Cell Line and Blockade of TIGIT-CD155 Interaction

To determine cell binding activity of chimeric antibodies, CHO-K1 cells expressing human and cynomolgus TIGIT were incubated first with antibodies (concentrations ranging from 1 pM to 100 nM) produced in Example 2, and then with fluorophore-labeled secondary polyclonal antibody against human Fc. To determine the activity of chimeric antibodies to block the interaction between TIGIT and CD155, 10 μg/mL of biotinylated CD155-Fc protein (Acrobiosystems) was added to the mixture of chimeric antibodies (concentrations ranging from 0.002 nM to 300 nM) and human TIGIT-CHO-K1 cells. Bound CD155-Fc was detected with fluorophore-labeled streptavidin. Binding and blocking activities of anti-TIGIT chimeric antibodies were summarized in Table 2. Most chimeric antibodies had similar or better binding and blocking activities than positive controls 4.1D3 and Hu1217-2-2, except TIGIT-52.

TABLE 2
Binding and blocking activities of anti-TIGIT chimeric antibodies

| | Binding EC50 (nM) to | | CD155 Blocking |
|---|---|---|---|
| | TIGIT cell lines | | IC50 (nM) |
| mAb | human | cynomolgus | human |
| TIGIT-3 | 0.53 | 2 | 0.38 |
| TIGIT-6 | 0.42 | 2.64 | 0.57 |
| TIGIT-15 | 1.03 | 3.53 | 1.07 |
| TIGIT-18 | 0.76 | 1.45 | 1.23 |
| TIGIT-19 | 0.41 | 1.05 | 0.4 |
| TIGIT-52 | 8.43 | 7.68 | 13.86 |
| TIGIT-63 | 1.12 | 1.92 | 2.32 |
| TIGIT-87 | 0.4 | 0.81 | 0.53 |
| TIGIT-94 | 0.51 | 0.74 | 0.72 |
| TIGIT-100 | 0.55 | >1000 | 0.82 |
| 4.1D3 | 0.95 | 2.77 | 1.4 |
| Hu1217-2-2 | 0.46 | >1000 | 0.81 |

### Affinity Determination by Surface Plasmon Resonance (SPR)

The binding affinity of chimeric antibodies to human and cynomolgus TIGIT proteins was determined by surface plasmon resonance (SPR) on a BIAcore T200 instrument (GE Healthcare). The experiment was carried out as follows: antibodies were captured onto the sensorchip pre-coated with Goat-anti-human pAb (40 μg/mL) through the interaction between polyclonal antibody and human Fc. Increasing concentrations (320, 160, 80, 40, 20, 10, 5, 2.5, 1.25 nM) of His-tagged human or cynomolgus TIGIT flowed over the sensorchip surface, and were allowed to bind the antibody for 100 s followed by injection of running buffer to allow dissociation of the complex. On-rate (ka) and off-rate (ka) were calculated based on association and dissociation curves, and were used to calculate the equilibrium dissociation constant (K_{D}). The affinity results were summarized in Table 3.

TABLE 3
Monovalent binding affinity of anti-TIGIT antibodies

| | binding to human TIGIT | | | binding to cynomolgus TIGIT | | |
|---|---|---|---|---|---|---|
| mAb | kₐ (1/Ms) | k_{d} (1/s) | K_{D} (M) | Kₐ (1/Ms) | k_{d} (1/s) | K_{D} (M) |
| TIGIT-3 | 7.30E+05 | 5.90E−05 | 8.10E−11 | 1.10E+05 | 2.00E−04 | 1.80E−09 |
| TIGIT-6 | 5.60E+05 | 1.30E−04 | 2.40E−10 | 9.00E+04 | 4.90E−03 | 5.40E−08 |
| TIGIT-15 | 8.60E+05 | 1.60E−04 | 1.90E−10 | 1.80E+05 | 2.00E−04 | 1.10E−09 |
| TIGIT-18 | 4.30E+05 | 9.50E−05 | 2.20E−10 | 2.60E+05 | 1.10E−03 | 4.40E−09 |
| TIGIT-19 | 1.50E+06 | 1.00E−04 | 7.10E−11 | 7.30E+05 | 6.60E−05 | 9.10E−11 |
| TIGIT-52 | 4.40E+05 | 7.40E−05 | 1.70E−10 | 2.20E+05 | 6.10E−05 | 2.80E−10 |
| TIGIT-63 | 2.50E+05 | 6.20E−04 | 2.50E−09 | 1.40E+05 | 4.90E−04 | 3.40E−09 |
| TIGIT-87 | 2.00E+06 | 1.20E−03 | 6.30E−10 | 1.30E+06 | 6.30E−02 | 4.70E−08 |
| TIGIT-94 | 1.20E+06 | 1.90E−03 | 1.60E−09 | 9.80E+05 | 7.50E−03 | 7.70E−09 |
| TIGIT-100 | 2.20E+06 | 1.40E−02 | 6.60E−09 | 4.20E+05 | 1.20E−02 | 2.90E−08 |
| 4.1D3 | 6.60E+05 | 4.30E−05 | 6.50E−11 | 2.00E+05 | 1.10E−03 | 5.40E−09 |
| Hu1217-2-2 | 1.20E+06 | 1.30E−04 | 1.10E−10 | 1.60E+05 | 4.40E−02 | 2.80E−07 |

### TIGIT/CD155 Blockade Reporter Assay

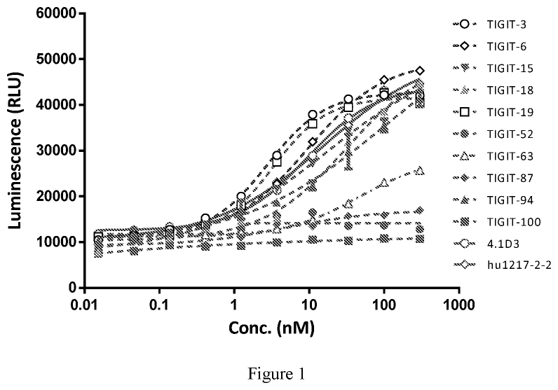

Reporter assay was performed using TIGIT/CD155 blockade reporter cell line (GenScript). Briefly, effector cells (TIGIT-over expressing NFAT-Luc/Jurkat cell line) were plated overnight and then incubated with anti-TIGIT antibodies (concentration ranging from 0.002 nM to 300 nM), followed by addition of CD155 aAPC/CHO-K1 cells. After 6 hours' induction at 37° C., 5% CO₂, Bio-Glo™ Luciferase Assay Reagent was added and luminescence was measured. Of all 10 chimeric antibodies, TIGIT-52, TIGIT-87 and TIGIT-100 showed low blocking activity, whereas the rest of the antibodies showed obvious blocking activity (FIG. 1 and Table 4). Dose-response relationship was fitted to three parameter dose-response curve using Prism 6 (GraphPad Software, San Diego, CA). Of all chimeric antibodies, TIGIT-3, TIGIT-6 and TIGIT-19 showed comparable or slightly superior blocking activity to that of 4.1D3 and Hu1217-2-2.

TABLE 4
TIGIT/CD155 blockade reporter assay result.

| mAb | Bottom (RLU) | Top (RLU) | EC50 (nM) |
|---|---|---|---|
| TIGIT-3 | 11194 | 42758 | 2.77 |
| TIGIT-6 | 10879 | 49765 | 9.42 |
| TIGIT-15 | 7720 | 51896 | 17.43 |
| TIGIT-18 | 10358 | 50641 | 31.3 |
| TIGIT-19 | 11140 | 41602 | 3.28 |
| TIGIT-52 | 11784 | 14166 | >100 |
| TIGIT-63 | 11161 | 28672 | 46.81 |
| TIGIT-87 | 9305 | 17037 | >100 |
| TIGIT-94 | 9955 | 59846 | 92.78 |
| TIGIT-100 | 4521 | 11714 | >100 |
| 4.1D3 | 12562 | 44193 | 9.28 |
| Hu1217-2-2 | 10478 | 51680 | 15.56 |

### Example 4: Humanization of Anti-TIGIT mAbs

The positioning of the three heavy chain CDR (HC-CDR) sequences in the VH chain and the three light chain CDR (LC-CDR) sequences in the VL chain were defined according to Abm numbering system (for HCDR1) and Kabat numbering system (for HCDR2 and 3, LCDR1-3). The sequences of the CDRs noted herein are provided in Table 12. Three anti-TIGIT mAbs, namely TIGIT-3, TIGIT-6 and TIGIT-19, were selected for humanization using CDR grafting technology (see, e.g., U.S. Pat. No. 5,225,539). Briefly, the variable chain sequences of the murine antibodies were compared to human heavy chain and K chain germline sequences. The most appropriate human frameworks on which to build the CDR grafted heavy and light chains were identified (Table 5). At first, straight graft was carried out. In case of affinity loss of these straight-graft antibodies, several framework residues were mutated back to their murine counterparts (i.e., backmutation) to restore the binding affinity of antibody. In the meantime, some CDR residues were either mutated to their human counterparts to increase the humanness of the antibody or to some other residues to remove potential post-translational modification spot or both. These humanized VH and VL sequences were used to construct full-length IgG heavy chains and light chains. Heavy chain and light chain plasmids were prepared and used for IgG production by HEK293 cells. Supernatants of transfected HEK293 cells were collected and were subjected to SPR assay as described in EXAMPLE 3. Mutations that did not significantly reduce the binding affinity were kept while mutations that did abolish the binding activity were abandoned.

TABLE 5
Human acceptor sequence accession numbers

| mAb | VH acceptor | Vκ acceptor |
|---|---|---|
| TIGIT-3 | BAC02278 | QTX15665 |
| TIGIT-6 | QVG74320 | ACY78416 |
| TIGIT-19 | AXA20212 | ABA70776 |

### Humanization of TIGIT-3

Four rounds of humanization design were carried out for TIGIT-3. For the humanization of TIGIT-3 VH sequence (hereafter referred to as 3VH), several back mutations were introduced to the straight-graft sequence, generating the humanized VH sequence of 3VH3 (SEQ ID NO: 22). From Table 6, pairing 3VH3 with mouse VL sequence (hereafter referred to as 3VL) did not affect the binding affinity (Table 6, SPR experiment 1). Making germline mutations which changes several mouse amino acid residues to their human germline counterparts, i.e., D54S and P61A in HCDR2 (3VH3.M4, SEQ ID NO: 23), further increased the humanness of the sequence, and did not reduce the binding affinity to human TIGIT (Table 6, SPR experiment 2). Straightly grafting mouse 3VL CDRs to the Vκ acceptor made the sequence of 3VL1 (SEQ ID NO: 24). Pairing it with humanized 3VH3 resulted in about 100-fold affinity decrease. On the other hand, making germline mutations to 3VL1 in LCDR1 (i.e., 3VL1.M1, SEQ ID NO: 25) and LCDR2 (i.e., 3VL1.M3, SEQ ID NO: 26 and 3VL1.M4, SEQ ID NO: 27) did not further reduce the binding affinity significantly (Table 6, SPR experiment 3), therefore, M1 and M4 mutations were combined to further increase the humanness of VL sequence. In the meantime, back mutations were also introduced to 3VL1, generating the sequences of 3VL4.M6 (SEQ ID NO: 28) and 3VL5.M6 (SEQ ID NO: 29). These humanized VLs were paired with 3VH3.M4. The binding affinity to human TIGIT was restored, with as little as 2-fold affinity decrease (Table 6, SPR experiment 4).

TABLE 6
Binding affinity of reshaped TIGIT-3 antibodies

| | antibody | | | | |
|---|---|---|---|---|---|
| experiment # | VH | VL | kₐ (1/Ms) | k_{d} (1/s) | K_{D} (M) |
| 1 | 3VH | 3VL | 5.1E+05 | 1.2E−04 | 2.4E−10 |
| | 3VH3 | 3VL | 5.8E+05 | 1.5E−04 | 2.5E−10 |
| 2 | 3VH3 | 3VL | 5.2E+05 | 2.5E−04 | 4.8E−10 |
| | 3VH3.M4 | 3VL | 4.5E+05 | 1.6E−04 | 3.6E−10 |
| 3 | 3VH3 | 3VL1 | 2.1E+05 | 7.6E−03 | 3.6E−08 |
| | 3VH3 | 3VL1.M1 | 2.3E+05 | 3.8E−03 | 1.7E−08 |
| | 3VH3 | 3VL1.M3 | 1.9E+05 | 8.9E−03 | 4.6E−08 |
| | 3VH3 | 3VL1.M4 | 2.3E+05 | 1.2E−02 | 5.5E−08 |
| 4 | 3VH | 3VL | 6.1E+05 | 1.7E−04 | 2.8E−10 |
| | 3VH3.M4 | 3VL4.M6 | 8.2E+05 | 4.6E−04 | 5.6E−10 |
| | 3VH3.M4 | 3VL5.M6 | 8.7E+05 | 4.3E−04 | 5.0E−10 |

### Humanization of TIGIT-6

Two rounds of humanization design were carried out for TIGIT-6. For the humanization of TIGIT-6 VH sequence (hereafter referred to as 6VH), several back mutations were introduced to the straight-graft sequence, generating the humanized VH sequence of 6VH5 (SEQ ID NO: 30) and 6VH6 (SEQ ID NO: 31). Straightly grafting mouse 6VL CDRs to the Vκ acceptor made the sequence of 6VL1 (SEQ ID NO: 32). From Table 7, pairing 6VH5 or 6VH6 with 6VL1 sequence did not affect the binding affinity significantly (SPR experiment 1). Making germline mutations in HCDR1 (6VH6.M1, SEQ ID NO: 33), HCDR2 (6VH6.M3, SEQ ID NO: 34) and in LCDR1 (6VL1.M1, SEQ ID NO: 35), further increases the humanness of the sequence, but did not reduce the binding affinity to human TIGIT significantly (Table 7, SPR experiment 1). To remove the potential asparagine deamidation Asn-Gly motif of HCDR2 of 6VH6.M3, N55Q or G56A mutations were also constructed (6VH6.M4, SEQ ID NO: 36 and 6VH6.M5, SEQ ID NO: 37, respectively). Germline mutations as designed in HCDR1 of 6VH6.M1 were combined 6VH6.M4 and 6VH6.M5 to further increase the humanness of the VH sequences, generating the sequences of 6VH6.M6 (SEQ ID NO: 38) and 6VH6.M7 (SEQ ID NO: 39). The binding affinity of these clones were determined, no significant affinity loss was observed (Table 7, SPR experiment 2).

TABLE 7
Humanization of TIGIT-6

| | antibody | | | | |
|---|---|---|---|---|---|
| experiment # | VH | VL | kₐ (1/Ms) | k_{d} (1/s) | K_{D} (M) |
| 1 | 6VH | 6VL | 5.6E+05 | 5.3E−05 | 9.4E−11 |
| | 6VH5 | 6VL1 | 3.1E+05 | 1.1E−04 | 3.5E−10 |
| | 6VH6 | 6VL1 | 4.7E+05 | 1.4E−04 | 3.0E−10 |
| | 6VH6.M1 | 6VL1 | 4.1E+05 | 2.6E−04 | 6.3E−10 |
| | 6VH6.M3 | 6VL1 | 3.5E+05 | 1.2E−04 | 3.5E−10 |
| | 6VH6.M4 | 6VL1 | 2.3E+05 | 1.5E−04 | 6.5E−10 |
| | 6VH6 | 6VL1.M1 | 5.3E+05 | 1.3E−04 | 2.4E−10 |
| 2 | 6VH | 6VL | 4.2E+05 | 1.2E−04 | 2.9E−10 |
| | 6VH6.M3 | 6VL1.M1 | 3.2E+05 | 1.6E−04 | 5.0E−10 |
| | 6VH6.M4 | 6VL1.M1 | 2.8E+05 | 9.8E−05 | 3.4E−10 |
| | 6VH6.M5 | 6VL1.M1 | 3.0E+05 | 2.2E−04 | 7.1E−10 |
| | 6VH6.M6 | 6VL1.M1 | 2.7E+05 | 1.7E−04 | 6.4E−10 |
| | 6VH6.M7 | 6VL1.M1 | 3.0E+05 | 2.3E−04 | 7.5E−10 |

### Humanization of TIGIT-19

Two rounds of humanization design were carried out for TIGIT-19. For the humanization of TIGIT-19 VH sequence (hereafter referred to as 19VH), several back mutations were introduced to the straight-graft sequence, generating the humanized VH sequence of 19VH4 (SEQ ID NO: 40). Straightly grafting mouse 19VL CDRs to the Vκ acceptor made the sequence of 19VL1 (SEQ ID NO: 41). From Table 8, pairing 19VH4 with 19VL1 sequence slightly improved the binding affinity (˜4-fold). Making germline mutations in LCDR2 (19VL1.M4, SEQ ID NO: 42) did not change the binding affinity to human TIGIT; whereas making further germline mutations LCDR1 (19VL1.M5, SEQ ID NO: 43, 19VL1.M6, SEQ ID NO: 44 and 19VL1.M7, SEQ ID NO: 45) slight reduced the binding affinity to human TIGIT (Table 8, SPR experiment 2). Making germline mutations in HCDRs made the sequences of 19VH4.M4 (SEQ ID NO: 46), 19VH4.M5 (SEQ ID NO: 47), 19VH4.M6 (SEQ ID NO: 48) and 19VH4.M7 (SEQ ID NO: 49). Compared to 19VH4, the binding affinity of these germlined clones was also slighted reduced. To remove the potential acid labile Asp-Pro motif in HCDR2, P53A mutation was introduced to 19VH4.M6 and 19VH4.M7, resulting in the sequences of 19VH4.M9 (SEQ ID NO: 50) and 19VH4.M10 (SEQ ID NO: 51). The binding affinity of 19VH4.M9 was comparable to that of 19VH4.M6 (Table 8, SPR experiment 2), suggesting P53A mutation did not reduce the binding affinity.

TABLE 8
Humanization of TIGIT-19

| | antibody | | | | |
|---|---|---|---|---|---|
| experiment # | VH | VL | kₐ (1/Ms) | k_{d} (1/s) | K_{D} (M) |
| 1 | 19VH | 19VL | 1.4E+06 | 9.7E−05 | 7.1E−11 |
| | 19VH4 | 19VL1 | 1.28E+06 | 2.51E−05 | 1.96E−11 |
| 2 | 19VH | 19VL | 1.2E+06 | 1.5E−04 | 1.2E−10 |
| | 19VH4 | 19VL1 | 1.2E+06 | 3.3E−05 | 2.8E−11 |
| | 19VH4 | 19VL1.M4 | 1.2E+06 | 2.7E−05 | 2.3E−11 |
| | 19VH4 | 19VL1.M5 | 1.1E+06 | 8.7E−05 | 8.1E−11 |
| | 19VH4 | 19VL1.M6 | 1.0E+06 | 1.5E−04 | 1.5E−10 |
| | 19VH4 | 19VL1.M7 | 1.3E+06 | 1.7E−04 | 1.3E−10 |
| | 19VH4.M4 | 19VL1 | 1.1E+06 | 1.3E−04 | 1.2E−10 |
| | 19VH4.M5 | 19VL1 | 1.3E+06 | 2.0E−04 | 1.6E−10 |
| | 19VH4.M6 | 19VL1 | 1.0E+06 | 2.1E−04 | 2.0E−10 |
| | 19VH4.M7 | 19VL1 | 1.1E+06 | 3.2E−04 | 3.0E−10 |
| | 19VH4.M9 | 19VL1 | 1.1E+06 | 2.3E−04 | 2.2E−10 |

### Example 5: In Vitro Characterization of Humanized Anti-TIGIT Antibodies

Several humanized antibodies were selected and produced as described in EXAMPLE 2. The purified antibodies were characterized in vitro using the following assays.

### Affinity Determination by SPR

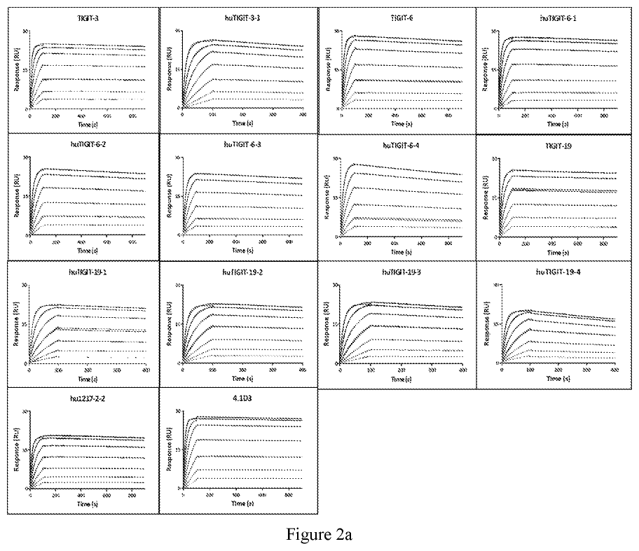

The binding affinity of humanized antibodies to human and cynomolgus TIGIT proteins was determined by SPR as described in EXAMPLE 3. The SPR sensorgrams were shown in FIG. 2 and affinity results were summarized in Table 9. For all anti-TIGIT antibodies, there is at least one humanized antibody that retained human TIGIT binding affinity. However, for TIGIT-3, the mono-valent cynomolgus TIGIT-binding affinity was significantly reduced. For TIGIT-6, the mono-valent cynomolgus TIGIT-binding affinity was much lower than the human TIGIT-binding affinity for both mouse and humanized antibodies. This is also true for Hu1217-2-2 and 4.1D3.

TABLE 9
Monovalent binding affinity of chimeric and humanized anti-TIGIT antibodies

| | | heavy chain | light chain | binding to human TIGIT | | | binding to cynomolgus TIGIT | | |
|---|---|---|---|---|---|---|---|---|---|
| mAb | VH + VL | SEQ ID | SEQ ID | kₐ (1/Ms) | k_{d} (1/s) | K_{D} (M) | kₐ (1/Ms) | k_{d} (1/s) | K_{D} (M) |
| TIGIT-3 | 3VH + 3VL | 52 | 53 | 6.3E+05 | 4.7E−05 | 7.5E−11 | 1.2E+05 | 5.3E−05 | 4.5E−10 |
| huTIGIT-3-1 | 3VH3.M4 + 3VL4.M6 | 54 | 55 | 5.7E+05 | 3.1E−04 | 5.4E−10 | 1.2E+05 | 2.2E−03 | 1.8E−08 |
| TIGIT-6 | 6VH + 6VL | 56 | 57 | 5.2E+05 | 9.0E−05 | 1.7E−10 | 1.2E+05 | 4.6E−03 | 3.9E−08 |
| huTIGIT-6-1 | 6VH6.M4 + 6VL1.M1 | 58 | 62 | 2.6E+05 | 5.6E−05 | 2.1E−10 | 5.9E+04 | 4.4E−03 | 7.5E−08 |
| huTIGIT-6-2 | 6VH6.M5 + 6VL1.M1 | 59 | 62 | 3.5E+05 | 9.6E−05 | 2.7E−10 | 6.9E+04 | 7.4E−03 | 1.1E−07 |
| huTIGIT-6-3 | 6VH6.M6 + 6VL1.M1 | 60 | 62 | 3.4E+05 | 9.8E−05 | 2.9E−10 | 6.1E+04 | 7.8E−03 | 1.3E−07 |
| huTIGIT-6-4 | 6VH6.M7 + 6VL1.M1 | 61 | 62 | 3.3E+05 | 1.9E−04 | 5.8E−10 | 4.7E+04 | 1.3E−02 | 2.6E−07 |
| TIGIT-19 | 19VH + 19VL | 63 | 64 | 1.5E+06 | 5.9E−05 | 4.0E−11 | 1.0E+06 | 3.2E−05 | 3.2E−11 |
| huTIGIT-19-1 | 19VH4.M9 + 19VL1.M4 | 65 | 67 | 1.1E+06 | 1.9E−04 | 1.6E−10 | 7.9E+05 | 3.7E−05 | 4.7E−11 |
| huTIGIT-19-2 | 19VH4.M9 + 19VL1.M7 | 65 | 68 | 1.1E+06 | 2.1E−04 | 1.9E−10 | 9.5E+05 | 5.2E−05 | 5.5E−11 |
| huTIGIT-19-3 | 19VH4.M10 + 19VL1.M4 | 66 | 67 | 1.1E+06 | 3.0E−04 | 2.8E−10 | 7.6E+05 | 4.1E−05 | 5.4E−11 |
| huTIGIT-19-4 | 19VH4.M10 + 19VL1.M7 | 66 | 68 | 1.2E+06 | 5.9E−04 | 4.9E−10 | 8.4E+05 | 7.3E−05 | 8.7E−11 |
| Hu1217-2-2 | Not applicable | | | 1.0E+06 | 5.1E−05 | 5.1E−11 | 1.3E+05 | 6.3E−02 | 4.7E−07 |
| 4.1D3 | | | | 6.3E+05 | 3.5E−05 | 5.5E−11 | 2.7E+05 | 8.3E−04 | 3.0E−09 |

### Binding of TIGIT Over-Expressing CHO Cell Line and Blockade of TIGIT-CD155 Interaction

To evaluate the accumulated strength of bi-valent binding interaction to both human and cynomolgus TIGIT, EC50 values of antibodies binding TIGIT over-expressing cell lines were determined by FACS as described in EXAMPLE 3. Antibodies' activity in blocking the interaction between human TIGIT and CD155 was carried out as described in EXAMPLE 3. Binding and blocking activities of chimeric and humanized anti-TIGIT antibodies were summarized in Table 10. For all anti-TIGIT antibodies, there is at least one humanized antibody that retained the human and cynomolgus TIGIT cell binding activity and human CD155 blocking activity. Most of the chimeric and humanized antibodies were shown to bind human and cynomolgus TIGIT over-expressing cell lines with similar binding EC50 values, except huTIGIT-6-4 which bound cynomolgus TIGIT over-expressing cell line with an EC50 value more than 10× higher than the EC50 value of binding human TIGIT over-expressing cell line. Chimeric and humanized TIGIT-3, TIGIT-6 and TIGIT-19 antibodies all showed significant CD155 blocking activity, especially TIGIT-19 which showed much better blocking activity compared to that of Hu1217-2-2 and 4.1D3.

TABLE 10
Binding and blocking activities of chimeric
and humanized anti-TIGIT antibodies

| | Binding EC50 (nM) to | | CD155 Blocking |
|---|---|---|---|
| | TIGIT cell lines | | IC50 (nM) |
| mAb | human | cynomolgus | human |
| TIGIT-3 | 0.3461 | 1.418 | 0.5581 |
| huTIGIT-3-1 | 0.5114 | 1.722 | 0.5021 |
| TIGIT-6 | 0.5807 | 2.308 | 0.5655 |
| huTIGIT-6-1 | 1.155 | 4.686 | 1.04 |
| huTIGIT-6-2 | 0.7445 | 4.982 | 0.9745 |
| huTIGIT-6-3 | 1.112 | 8.201 | 1.103 |
| huTIGIT-6-4 | 1.016 | 16.73 | 1.167 |
| TIGIT-19 | 0.3109 | 0.5239 | 0.2595 |
| huTIGIT-19-1 | 0.2936 | 0.4726 | 0.22 |
| huTIGIT-19-2 | 0.2558 | 0.4396 | 0.24 |
| huTIGIT-19-3 | 0.1961 | 0.3836 | 0.21 |
| huTIGIT-19-4 | 0.1717 | 0.4082 | 0.26 |
| Hu1217-2-2 | 0.6498 | 43.01 | 0.62 |
| 4.1D3 | 1.054 | 2.345 | 0.95 |

### TIGIT/CD155 Blockade Reporter Assay

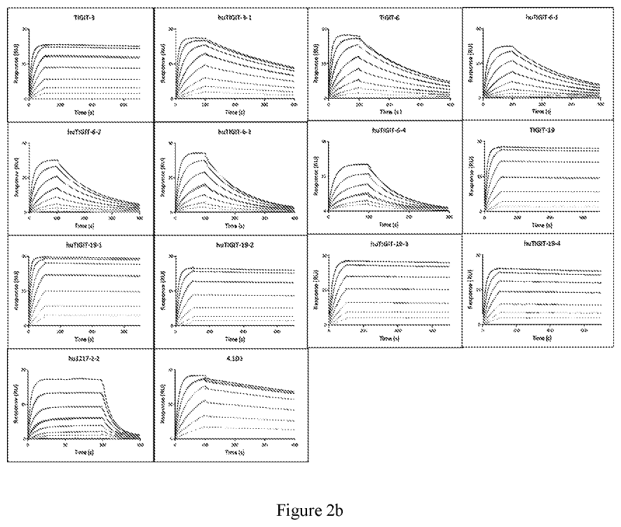

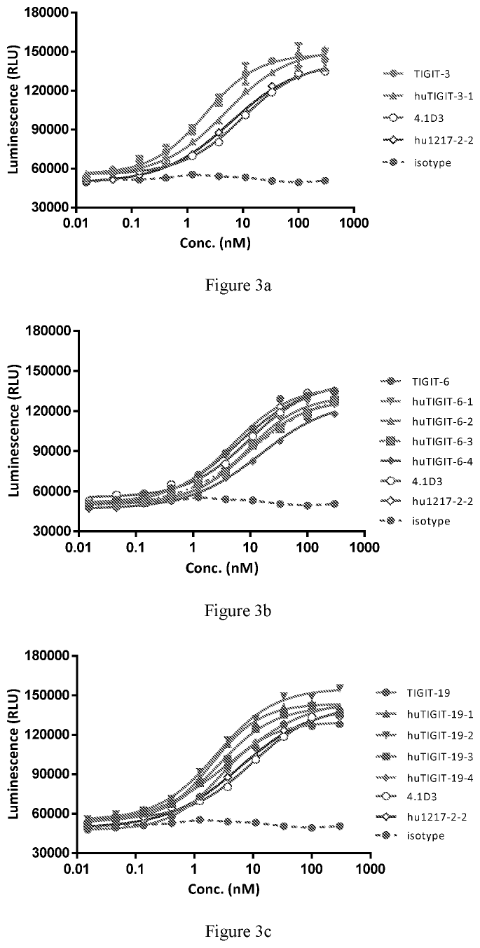

TIGIT/CD155 blockade reporter assay was carried out using chimeric and humanized antibodies as described in EXAMPLE 3. As can be seen from FIG. 3 and Table 11, humanized antibodies showed similar functional activity to that of chimeric antibodies. Humanized TIGIT-3, TIGIT-6 and TIGIT-19 antibodies all showed significant potency, especially humanized TIGIT-19 antibodies which showed slightly better potency compared to that of Hu1217-2-2 and 4.1D3, which is consistence with the result of CD155 blocking assay shown above.

TABLE 11
TIGIT/CD155 blockade reporter assay result
of chimeric and humanized antibodies.

| mAb | Bottom (RLU) | Top (RLU) | EC50 (nM) |
|---|---|---|---|
| TIGIT-3 | 55769 | 148160 | 2.10 |
| huTIGIT-3-1 | 54393 | 152061 | 4.92 |
| TIGIT-6 | 51389 | 138747 | 4.90 |
| huTIGIT-6-1 | 51180 | 131330 | 9.10 |
| huTIGIT-6-2 | 49833 | 128550 | 9.03 |
| huTIGIT-6-3 | 49371 | 135116 | 11.10 |
| huTIGIT-6-4 | 46792 | 128189 | 14.84 |
| TIGIT-19 | 47576 | 129732 | 2.75 |
| huTIGIT-19-1 | 53916 | 143724 | 2.05 |
| huTIGIT-19-2 | 55244 | 155389 | 2.39 |
| huTIGIT-19-3 | 53730 | 141701 | 2.65 |
| huTIGIT-19-4 | 51260 | 148216 | 4.28 |
| Hu1217-2-2 | 52926 | 163113 | 11.10 |
| 4.1D3 | 55332 | 142067 | 9.69 |

### Activation of CMV-Specific Human T Cells by Chimeric and Humanized Anti-TIGIT Antibodies

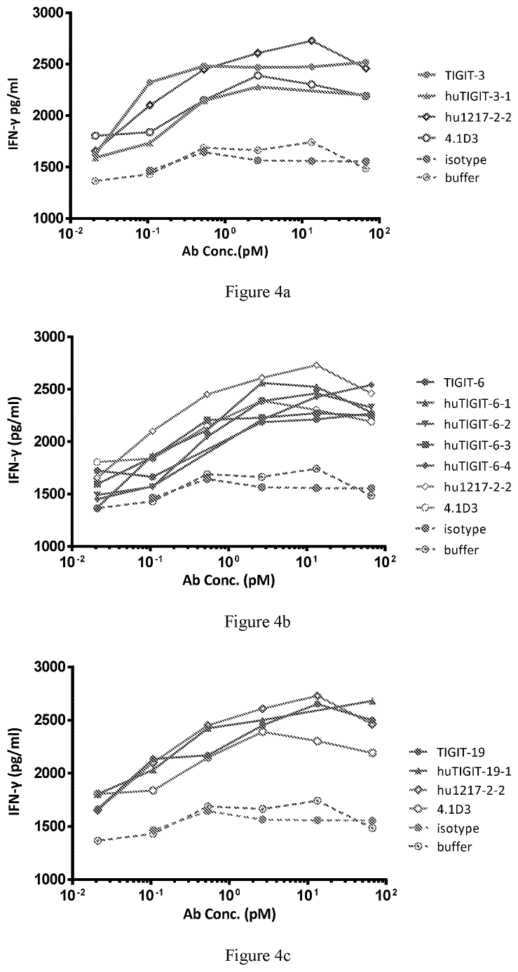

The functional activity of anti-TIGIT antibodies was further assessed using naturally derived human cytomegalovirus (CMV) specific T cells. Briefly, PBMCs from HLA-A*02:01 positive healthy donors were stimulated with 2 μg/mL nonamer peptide ₄₉₅NLVPMVATV₅₀₃ (residues 495-503 of CMV matrix protein pp65; hereafter referred to as CMVpp65₄₉₅₋₅₀₃ peptide) synthesized by GenScript in the AIM-V with 10% FBS for a week. After stimulation, cytotoxic T lymphocytes were enriched using CD8+ T Cell Isolation Kit (Miltenyi Biotech, Cat. #: 130-096-495). The CMV-specific T cells proliferated, and accounted for >20% of CD8+ T cells. More than 50% of CD8+ T Cell expressed TIGIT. TIGIT expression level was also elevated by 10-fold compared to unstimulated T cells. The purified T cells were used as effector cells, and incubated overnight in the AIM-V with 10% FBS with HLA-A*02:01 positive HepG2 cells pulsed with pp65 peptide (5 μg/mL, >4 hours) at roughly an effector-to-target ratio of 1:4 in the presence or absence (medium only) of anti-TIGIT antibodies or an isotype control. As shown in FIG. 4, all anti-TIGIT antibodies promoted IFN-γ secretion in a dose-dependent manner, suggesting all these antibodies are functional.

### ADCC Effector Function of Chimeric and Humanized Anti-TIGIT Antibodies

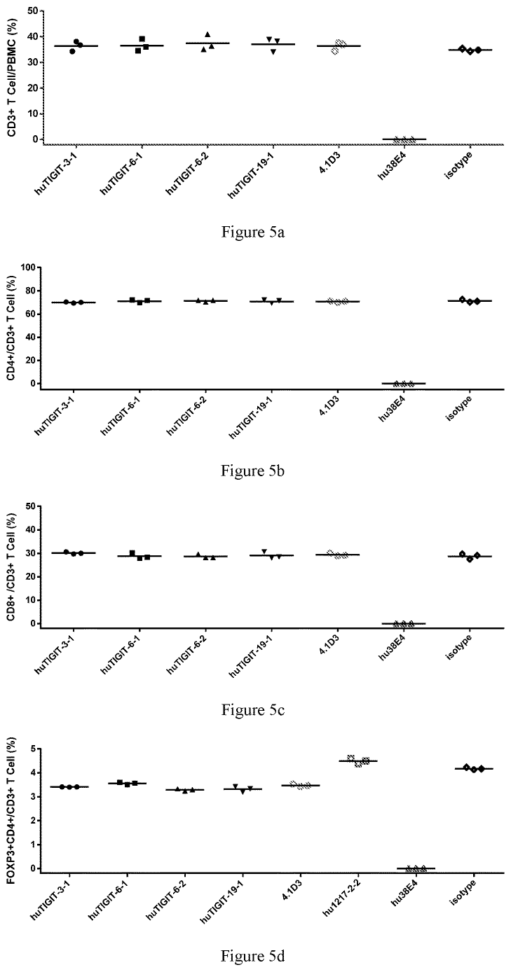

TIGIT is constitutively expressed by regulatory T cells (Treg). Tregs showed a higher proportion of TIGIT+ cells and higher number of TIGIT receptors per cell than other immune populations (Preillon J. et al. Restoration of T-cell effector function, eepletion of Tregs, and direct killing of tumor cells: the multiple mechanisms of action of a-TIGIT antagonist antibodies. Mol Cancer Ther. 2021: 20 (1): 121-131), so we measured the ability of anti-TIGIT mAbs to induce direct killing of Tregs through a flow cytometry-based antibody dependent cellular cytotoxicity (ADCC) assay. Briefly, human PBMCs from a healthy donor were stimulated with PHL-A (1 μg/mL, 72 hours) to upregulate TIGIT expression. The activated PBMCs were used as target cells. The effector cell line, NK92-CD16aVV cells, was generated in house by transfecting NK92 cells (ATCC) with CD16V158 (V158 allele) expression plasmid. The effector cells were co-cultured with the aforementioned PBMC target cells at an effector-to-target ratio of 1:1 for 48 hours in the absence (medium only) or presence of anti-TIGIT antibodies (30 μg/mL) or Hu1217-2-2/IgG1mf (Hu1217-2-2 antibody with an engineered Fc with no effector function, pat. No. WO2019129261A1) or an isotype control (30 μg/mL) or a positive control anti-CD3 antibody Hu38E4 (5 μg/mL, Biolegend). Compared to Hu1217-2-2/IgG1mf and human IgG1 isotype control, all anti-TIGIT antibodies could cause moderate reduction of Treg cells via ADCC. However, no significant killing of total T cells or CD8+ T cells or FoxP3-CD4+ T cells was observed (FIG. 5).

### Example 6: In Vivo Efficacy of Humanized Anti-TIGIT Antibodies

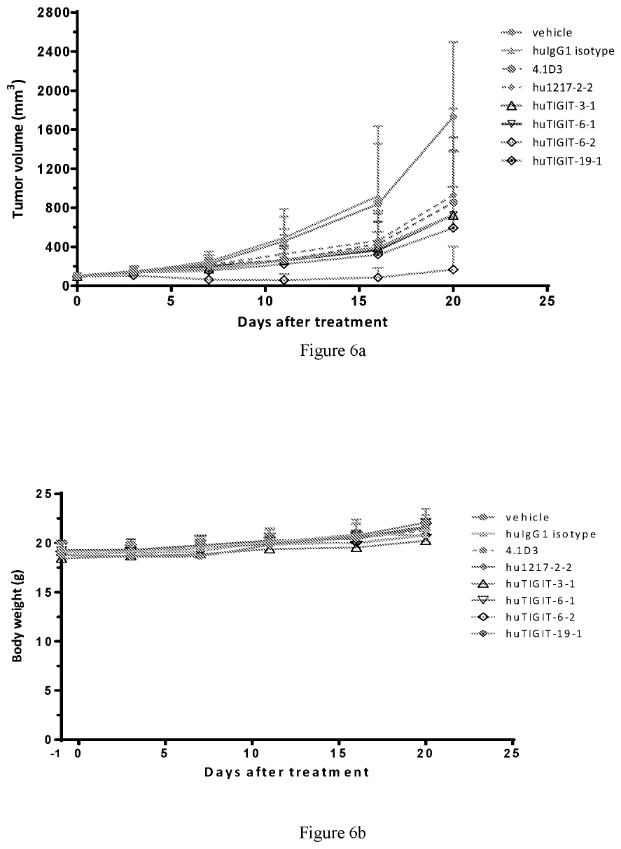

The in vivo efficacy of anti-TIGIT antibodies was studied using a syngeneic CT26 tumor model in Balb/c mice with human TIGIT gene knocked in. Briefly, CT26 tumor cells were cultured, and 5×10⁵ cells were injected subcutaneously at the flank of female human TIGIT knock-in Balb/c (Gempharmatech) mice (6-7 weeks of age). The sizes of tumors were measured using a caliper and tumor volumes were calculated as Length×Width×Width/2. When the average tumor volume reached ˜100 mm³, mice were randomized into groups of 5 or 6 mice and were treated with antibodies. Test articles were dosed once every 4 days at 0.4 mg/kg intraperitoneally. Body weights were measured throughout the study. The animals were sacrificed when the tumor volume reached 2000 mm³ or when the study ended (20 days post-treatment). As shown in FIG. 6a, even at suboptimal dose, anti-TIGIT antibodies to some extent inhibited tumor growth compared to isotype and vehicle controls, especially huTIGIT-6-2 which showed the highest tumor growth inhibition. The other 3 humanized antibodies also showed significant tumor growth inhibition activity. There was no sign of loss of body weight throughout the study (FIG. 6b).

### Sequences

TABLE 12

| SEQ | | |
|---|---|---|
| ID | | |
| NO: | Description | SEQUENCE |
| 1 | human TIGIT | MMTGTIETTGNISAEKGGSIILQCHLSSTTAQVTQVNWEQQ |
| | | DQLLAICNADLGWHISPSFKDRVAPGPGLGLTLQSLTVNDT |
| | | GEYFCIYHTYPDGTYTGRIFLEVLESSVAEHGARFQIP |
| 2 | TIGIT-3 VH | EVQLVESGGGLVQPGGSLKLSCAASGFTFSSYGMSWVRQT |
| | | PDKRLELVATIKSDGGSTNLPDSVKGRFTISRDNAKNTLHL |
| | | QMSSLKSEDTAMYYCVRGYFDYWGQGTSVTVSS |
| 3 | TIGIT-3 VL | DIVMTQSHKFMSTSVGDRVSITCKASQDVKTAVAWYQQK |
| | | PGQSPKLLIYWSSTRHTGVPDRFTGSRSLTDHTLTISSVQAE |
| | | DLALYYCQQYYSTPYTFGGGTKLELK |
| 4 | TIGIT-6 VH | QVQLKQSGPELVKPGASVKISCKTSGYTITENTMHWVKQS |
| | | HGKSLEWIGGINPNNGGTSYNQQFKGKATLTVDKSSSTAY |
| | | MELRSLTSEDSAVYYCTRSSYYRYDLDYWGQGTTVTVSS |
| 5 | TIGIT-6 VL | DIQMTQSHKFMSTSVGDRVSITCKASQDVKTALAWYQQK |
| | | PGQSPKLLIYSASYRYTGVPDRFTGSGSGTDFTFTISSVQAE |
| | | DLAVYYCHQHYTTPWTFGGGTKLEIK |
| 6 | TIGIT-15 VH | DVQLVESGGGLVQPGGSLKLSCAASGFTFSSYGMSWVRQ |
| | | TPDRRLELVATINNNGGSTYYPDSVKGRFTISRDNARNTLY |
| | | LQMSSLKSEDTAMYYCATRTGTEILGFDFWGQGTTLTVSS |
| 7 | TIGIT-15 VL | DIVLTQSHKFMSTSLGDRVSITCKASQDVSTAVAWYQQKP |
| | | GQSPKLLIYWASSRHTGVPDRFTGSRSGPDYTLTISSVQAE |
| | | DLALYYCQQQYSTPWTFGGGTKLEIK |
| 8 | TIGIT-18 VH | QVQLQQPGAELVRPGASVKLSCKASGYSFTSYWMNWVK |
| | | QRPGQGHEWIGMIHLSNSETRLNQNFLDKATLTVDKSSST |
| | | VYLQLSSPTSEDSAVYYCARERDYYGSSYFDYWGQGTTLT |
| | | VSS |
| 9 | TIGIT-18 VL | DIVMTQSPASLVVSLGQRATISCRTSQSVSTSSYSYMHWY |
| | | QQKPGQPPKLLIKNASNLESGVPARFSASGSGTDFTLNIHP |
| | | VEEEDAATYYCQHSWEIPRTFGGGTKLEIK |
| 10 | TIGIT-19 VH | QVQLQQPGPELVKPGTSVMLSCKASGYAFSRYSMYWVKQ |
| | | SHGKSLEWIAFIDPYNGGSTYNQRFRGKATLTVDKSSSTAY |
| | | MHLNTLTSDDSAVYYCARGGDYGWYLGVWGAGTTVTVS |
| | | S |
| 11 | TIGIT-19 VL | DIQMTQSPASLSASVGETVTITCRPSENIYTYLSWYQQKRG |
| | | KSPQLLVYNAKTLPENVPSRFSGSGSGTQFSLKIDSLQPEDF |
| | | GNYYCQHHYGNPLTFGAGTKLELK |
| 12 | TIGIT-52 VH | EVQLVESGGGLVEPGGSLTLSCVASGFTFSSFAMSWVRQT |
| | | PEKRLEWVATISNGGSYTYYPDTVKGRFTISRDNAKNTLFL |
| | | QMSSLRSEDTAMYYCARPYYGTTYGYYFDFWGQGTTLTV |
| | | SS |
| 13 | TIGIT-52 VL | QIVLTQSPASLAVSLGQRATISCRASESVDRNGNSFMHWY |
| | | QQKPGQPPKLLIYLASNLESGVPARFSGSGSRTDFTLTIDPV |
| | | EADDAATYYCQQNNEDPLTFGAGTKLELK |
| 14 | TIGIT-63 VH | DVQLVESGGGLVQPGGSLSLSCAASGFTFTDYYMNWVRQ |
| | | PPGKALEWLGFIRNKANGYTTEYSASVKGRFTISRDNSQSI |
| | | LYLQMNALRAEDSATYYCARSTAWYFDVWGTGTTLTVSS |
| 15 | TIGIT-63 VL | DIQMTQSPASLSASVGGTVTITCRASEHIYSYLAWYQQKQ |
| | | GKSPQLLVYNAKTLAEGVPSRFSGSGSGTQFSLKINSLQPK |
| | | DFGSYYCQHHYGNPLTFGAGTKLELK |
| 16 | TIGIT-87 VH | EVKLVESGPELVKPGASMKISCKASGYSFTGYTMNWVKQ |
| | | SHGKNLEWIGLINPYNGGTSYNQKFKGKATLTVDKSSSTA |
| | | YMELLSLTSEDSAVYYCARYANRFENWFAYWGQGTLVTV |
| | | SS |
| 17 | TIGIT-87 VL | DIQMTQSPASLSASVGETVTITCRASENIYSYLAWYQQKQG |
| | | KSPQLLVYNAKTLAEGVPSRFSGSGSGTQFSLKINSLQPED |
| | | FGSYYCQHHYGTPWTFGGGTKLEIK |
| 18 | TIGIT-94 VH | EVKLVESGGGLVKPGGSLKLSCAASGFTFSTYTMSWVRQT |
| | | PEKRLEWVATISSGGTYTHYPDSVKGRFTISRDNAKNTLYL |
| | | QMSSLKSEDTAMYYCTRWFLSLYAMDYWGQGTSVTVSS |
| 19 | TIGIT-94 VL | DIVMTQSTSSLSASLGDRVTISCRASQDINNFLNWYQQKPA |
| | | GTVDLLIYYTSRLHSGVPSRFSGSGSGTDYSLTISNLDQEDI |
| | | ATYFCQQGNTFPYTFGGGTRLEIK |
| 20 | TIGIT-100 VH | QVQLQQPGPELVKPGASVKISCKASGYSFTDYNMNWVKQ |
| | | SNGKSLEWIGVINPNSGTDNYNQKFKGKATLTVDQSSSTA |
| | | YMQLNSLTSEDSAVYFCARGVYFGNYFDSWGQGTTLTVS |
| | | S |
| 21 | TIGIT-100 VL | DIVMTQSPASLSASVGETVTITCRASENIYSSLAWYQQKQG |
| | | KSPQLLVFNAKTLTEGVPSRFSGSGSGTQFSLKINSLQPEDF |
| | | GSYYCQHHYGAPWTFGGGTKLEIK |
| 22 | 3VH3 | EVQLVESGGGLVQPGGSLRLSCAASGFTFSSYGMSWVRQA |
| | | PGKGLELVATIKSDGGSTNLPDSVKGRFTISRDNSKNTLYL |
| | | QMNSLRAEDTAVYYCVRGYFDYWGQGTTVTVSS |
| 23 | 3VH3.M4 | EVQLVESGGGLVQPGGSLRLSCAASGFTFSSYGMSWVRQA |
| | | PGKGLELVATIKSSGGSTNLADSVKGRFTISRDNSKNTLYL |
| | | QMNSLRAEDTAVYYCVRGYFDYWGQGTTVTVSS |
| 24 | 3VL1 | DIQMTQSPSSLSASVGDRVTITCKASQDVKTAVAWYQQKP |
| | | GKVPKLLIYWSSTRHTGVPSRFSGSGSGTDFTLTISSLQPED |
| | | VATYYCQQYYSTPYTFGGGTKVEIK |
| 25 | 3VL1.M1 | DIQMTQSPSSLSASVGDRVTITCRASQDIKTALAWYQQKPG |
| | | KVPKLLIYWSSTRHTGVPSRFSGSGSGTDFTLTISSLQPEDV |
| | | ATYYCQQYYSTPYTFGGGTKVEIK |
| 26 | 3VL1.M3 | DIQMTQSPSSLSASVGDRVTITCKASQDVKTAVAWYQQKP |
| | | GKVPKLLIYWASTRHSGVPSRFSGSGSGTDFTLTISSLQPED |
| | | VATYYCQQYYSTPYTFGGGTKVEIK |
| 27 | 3VL1.M4 | DIQMTQSPSSLSASVGDRVTITCKASQDVKTAVAWYQQKP |
| | | GKVPKLLIYWASTRQSGVPSRFSGSGSGTDFTLTISSLQPED |
| | | VATYYCQQYYSTPYTFGGGTKVEIK |
| 28 | 3VL4.M6 | DIQMTQSPSSLSASVGDRVTITCRASQDIKTALAWYQQKPG |
| | | KSPKLLIYWASTRQSGVPSRFSGSGSGTDFTLTISSLQPEDV |
| | | ATYYCQQYYSTPYTFGGGTKVEIK |
| 29 | 3VL5.M6 | DIQMTQSPSSLSASVGDRVTITCRASQDIKTALAWYQQKPG |
| | | QSPKLLIYWASTRQSGVPSRFSGSGSGTDFTLTISSLQPEDV |
| | | ATYYCQQYYSTPYTFGGGTKVEIK |
| 30 | 6VH5 | QVQLVQSGAEVKKPGASVKVSCKASGYTITENTMHWVRQ |
| | | APGQRLEWIGGINPNNGGTSYNQQFKGRATLTRDTSASTA |
| | | YMELSSLRSEDTAVYYCTRSSYYRYDLDYWGQGTTVTVS |
| | | S |
| 31 | 6VH6 | QVQLVQSGAEVKKPGASVKVSCKASGYTITENTMHWVRQ |
| | | APGQRLEWIGGINPNNGGTSYNQQFKGRATLTVDTSASTA |
| | | YMELSSLRSEDTAVYYCTRSSYYRYDLDYWGQGTTVTVS |
| | | S |
| 32 | 6VL1 | DIQMTQSPSSLSASVGDRVTITCKASQDVKTALAWYQQKP |
| | | GKAPKLLIYSASYRYTGVPSRFSGSGSGTDFTFTISSLQPEDI |
| | | ATYYCHQHYTTPWTFGGGTKLEIK |
| 33 | 6VH6.M1 | QVQLVQSGAEVKKPGASVKVSCKASGYTFTENAMHWVR |
| | | QAPGQRLEWIGGINPNNGGTSYNQQFKGRATLTVDTSAST |
| | | AYMELSSLRSEDTAVYYCTRSSYYRYDLDYWGQGTTVTV |
| | | SS |
| 34 | 6VH6.M3 | QVQLVQSGAEVKKPGASVKVSCKASGYTITENTMHWVRQ |
| | | APGQRLEWIGGINPNNGGTSYSQKFQGRATLTVDTSASTA |
| | | YMELSSLRSEDTAVYYCTRSSYYRYDLDYWGQGTTVTVS |
| | | S |
| 35 | 6VL1.M1 | DIQMTQSPSSLSASVGDRVTITCQASQDIKTALAWYQQKP |
| | | GKAPKLLIYSASYRYTGVPSRFSGSGSGTDFTFTISSLQPEDI |
| | | ATYYCHQHYTTPWTFGGGTKLEIK |
| 36 | 6VH6.M4 | QVQLVQSGAEVKKPGASVKVSCKASGYTITENTMHWVRQ |
| | | APGQRLEWIGGINPNQGGTSYSQKFQGRATLTVDTSASTA |
| | | YMELSSLRSEDTAVYYCTRSSYYRYDLDYWGQGTTVTVS |
| | | S |
| 37 | 6VH6.M5 | QVQLVQSGAEVKKPGASVKVSCKASGYTITENTMHWVRQ |
| | | APGQRLEWIGGINPNNAGTSYSQKFQGRATLTVDTSASTA |
| | | YMELSSLRSEDTAVYYCTRSSYYRYDLDYWGQGTTVTVS |
| | | S |
| 38 | 6VH6.M6 | QVQLVQSGAEVKKPGASVKVSCKASGYTFTENAMHWVR |
| | | QAPGQRLEWIGGINPNQGGTSYSQKFQGRATLTVDTSAST |
| | | AYMELSSLRSEDTAVYYCTRSSYYRYDLDYWGQGTTVTV |
| | | SS |
| 39 | 6VH6.M7 | QVQLVQSGAEVKKPGASVKVSCKASGYTFTENAMHWVR |
| | | QAPGQRLEWIGGINPNNAGTSYSQKFQGRATLTVDTSAST |
| | | AYMELSSLRSEDTAVYYCTRSSYYRYDLDYWGQGTTVTV |
| | | SS |
| 40 | 19VH4 | QVQLVQSGAEVKKPGASVKVSCKASGYAFSRYSMYWVR |
| | | QAPGQGLEWIAFIDPYNGGSTYNQRFRGKATLTVDTSISTA |
| | | YMELSRLRSDDTAVYYCARGGDYGWYLGVWGQGTLVTV |
| | | SS |
| 41 | 19VL1 | DIQMTQSPSSLSASVGDRVTITCRPSENIYTYLSWYQQKPG |
| | | KAPKLLIYNAKTLPEGVPSRFSGSGSGTDFTLTISSLQPEDF |
| | | ATYYCQHHYGNPLTFGQGTKVEIK |
| 42 | 19VL1.M4 | DIQMTQSPSSLSASVGDRVTITCRPSENIYTYLSWYQQKPG |
| | | KAPKLLIYNAKSLPSGVPSRFSGSGSGTDFTLTISSLQPEDF |
| | | ATYYCQHHYGNPLTFGQGTKVEIK |
| 43 | 19VL1.M5 | DIQMTQSPSSLSASVGDRVTITCRASQNIYTYLSWYQQKPG |
| | | KAPKLLIYNAKSLPSGVPSRFSGSGSGTDFTLTISSLQPEDF |
| | | ATYYCQHHYGNPLTFGQGTKVEIK |
| 44 | 19VL1.M6 | DIQMTQSPSSLSASVGDRVTITCRASQNIYSYLSWYQQKPG |
| | | KAPKLLIYNAKSLPSGVPSRFSGSGSGTDFTLTISSLQPEDF |
| | | ATYYCQHHYGNPLTFGQGTKVEIK |
| 45 | 19VL1.M7 | DIQMTQSPSSLSASVGDRVTITCRASQSIYSYLSWYQQKPG |
| | | KAPKLLIYNAKSLPSGVPSRFSGSGSGTDFTLTISSLQPEDF |
| | | ATYYCQHHYGNPLTFGQGTKVEIK |
| 46 | 19VH4.M4 | QVQLVQSGAEVKKPGASVKVSCKASGYAFSRYSMYWVR |
| | | QAPGQGLEWIAFIDPYSGGSTYNQRFRGKATLTVDTSISTA |
| | | YMELSRLRSDDTAVYYCARGGDYGWYLGVWGQGTLVTV |
| | | SS |
| 47 | 19VH4.M5 | QVQLVQSGAEVKKPGASVKVSCKASGYAFSRYSMYWVR |
| | | QAPGQGLEWIAFIDPYNGGSTYAQKFQGKATLTVDTSISTA |
| | | YMELSRLRSDDTAVYYCARGGDYGWYLGVWGQGTLVTV |
| | | SS |
| 48 | 19VH4.M6 | QVQLVQSGAEVKKPGASVKVSCKASGYTFTRYSMYWVR |
| | | QAPGQGLEWIAFIDPYSGGSTYNQRFRGKATLTVDTSISTA |
| | | YMELSRLRSDDTAVYYCARGGDYGWYLGVWGQGTLVTV |
| | | SS |
| 49 | 19VH4.M7 | QVQLVQSGAEVKKPGASVKVSCKASGYTFTRYSMYWVR |
| | | QAPGQGLEWIAFIDPYSGGSTYAQKFQGKATLTVDTSISTA |
| | | YMELSRLRSDDTAVYYCARGGDYGWYLGVWGQGTLVTV |
| | | SS |
| 50 | 19VH4.M9 | QVQLVQSGAEVKKPGASVKVSCKASGYTFTRYSMYWVR |
| | | QAPGQGLEWIAFIDAYSGGSTYNQRFRGKATLTVDTSISTA |
| | | YMELSRLRSDDTAVYYCARGGDYGWYLGVWGQGTLVTV |
| | | SS |
| 51 | 19VH4.M10 | QVQLVQSGAEVKKPGASVKVSCKASGYTFTRYSMYWVR |
| | | QAPGQGLEWIAFIDAYSGGSTYAQKFQGKATLTVDTSISTA |
| | | YMELSRLRSDDTAVYYCARGGDYGWYLGVWGQGTLVTV |
| | | SS |
| 52 | TIGIT-3 chimeric | EVQLVESGGGLVQPGGSLKLSCAASGFTFSSYGMSWVRQT |
| | heavy chain | PDKRLELVATIKSDGGSTNLPDSVKGRFTISRDNAKNTLHL |
| | | QMSSLKSEDTAMYYCVRGYFDYWGQGTSVTVSSASTKGP |
| | | SVFPLAPSSKSTSGGTAALGCLVKDYFPEPVTVSWNSGALT |
| | | SGVHTFPAVLQSSGLYSLSSVVTVPSSSLGTQTYICNVNHK |
| | | PSNTKVDKRVEPKSCDKTHTCPPCPAPEAEGAPSVFLFPPK |
| | | PKDTLMISRTPEVTCVVVDVSHEDPEVKFNWYVDGVEVH |
| | | NAKTKPREEQYNSTYRVVSVLTVLHQDWLNGKEYKCKVS |
| | | NKALPSSIEKTISKAKGQPREPQVYTLPPSREEMTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSP |
| | | GK |
| 53 | TIGIT-3 chimeric | DIVMTQSHKFMSTSVGDRVSITCKASQDVKTAVAWYQQK |
| | light chain | PGQSPKLLIYWSSTRHTGVPDRFTGSRSLTDHTLTISSVQAE |
| | | DLALYYCQQYYSTPYTFGGGTKLELKRTVAAPSVFIFPPSD |
| | | EQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |
| 54 | 3VH3.M4 heavy chain | EVQLVESGGGLVQPGGSLRLSCAASGFTFSSYGMSWVRQA |
| | | PGKGLELVATIKSSGGSTNLADSVKGRFTISRDNSKNTLYL |
| | | QMNSLRAEDTAVYYCVRGYFDYWGQGTTVTVSSASTKGP |
| | | SVFPLAPSSKSTSGGTAALGCLVKDYFPEPVTVSWNSGALT |
| | | SGVHTFPAVLQSSGLYSLSSVVTVPSSSLGTQTYICNVNHK |
| | | PSNTKVDKRVEPKSCDKTHTCPPCPAPEAEGAPSVFLFPPK |
| | | PKDTLMISRTPEVTCVVVDVSHEDPEVKFNWYVDGVEVH |
| | | NAKTKPREEQYNSTYRVVSVLTVLHQDWLNGKEYKCKVS |
| | | NKALPSSIEKTISKAKGQPREPQVYTLPPSREEMTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSP |
| | | GK |
| 55 | 3VL4.M6 light chain | DIQMTQSPSSLSASVGDRVTITCRASQDIKTALAWYQQKPG |
| | | KSPKLLIYWASTRQSGVPSRFSGSGSGTDFTLTISSLQPEDV |
| | | ATYYCQQYYSTPYTFGGGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 56 | TIGIT-6 chimeric | QVQLKQSGPELVKPGASVKISCKTSGYTITENTMHWVKQS |
| | heavy chain | HGKSLEWIGGINPNNGGTSYNQQFKGKATLTVDKSSSTAY |
| | | MELRSLTSEDSAVYYCTRSSYYRYDLDYWGQGTTVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKRVEPKSCDKTHTCPPCPAPEAEGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPSSIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| 57 | TIGIT-6 chimeric | DIQMTQSHKFMSTSVGDRVSITCKASQDVKTALAWYQQK |
| | light chain | PGQSPKLLIYSASYRYTGVPDRFTGSGSGTDFTFTISSVQAE |
| | | DLAVYYCHQHYTTPWTFGGGTKLEIKRTVAAPSVFIFPPSD |
| | | EQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |
| 58 | 6VH6.M4 heavy chain | QVQLVQSGAEVKKPGASVKVSCKASGYTITENTMHWVRQ |
| | | APGQRLEWIGGINPNQGGTSYSQKFQGRATLTVDTSASTA |
| | | YMELSSLRSEDTAVYYCTRSSYYRYDLDYWGQGTTVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQTY |
| | | ICNVNHKPSNTKVDKRVEPKSCDKTHTCPPCPAPEAEGAPS |
| | | VFLFPPKPKDTLMISRTPEVTCVVVDVSHEDPEVKFNWYV |
| | | DGVEVHNAKTKPREEQYNSTYRVVSVLTVLHQDWLNGKE |
| | | YKCKVSNKALPSSIEKTISKAKGQPREPQVYTLPPSREEMT |
| | | KNQVSLTCLVKGFYPSDIAVEWESNGQPENNYKTTPPVLD |
| | | SDGSFFLYSKLTVDKSRWQQGNVFSCSVMHEALHNHYTQ |
| | | KSLSLSPGK |
| 59 | 6VH6.M5 heavy chain | QVQLVQSGAEVKKPGASVKVSCKASGYTITENTMHWVRQ |
| | | APGQRLEWIGGINPNNAGTSYSQKFQGRATLTVDTSASTA |
| | | YMELSSLRSEDTAVYYCTRSSYYRYDLDYWGQGTTVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQTY |
| | | ICNVNHKPSNTKVDKRVEPKSCDKTHTCPPCPAPEAEGAPS |
| | | VFLFPPKPKDTLMISRTPEVTCVVVDVSHEDPEVKFNWYV |
| | | DGVEVHNAKTKPREEQYNSTYRVVSVLTVLHQDWLNGKE |
| | | YKCKVSNKALPSSIEKTISKAKGQPREPQVYTLPPSREEMT |
| | | KNQVSLTCLVKGFYPSDIAVEWESNGQPENNYKTTPPVLD |
| | | SDGSFFLYSKLTVDKSRWQQGNVFSCSVMHEALHNHYTQ |
| | | KSLSLSPGK |
| 60 | 6VH6.M6 heavy chain | QVQLVQSGAEVKKPGASVKVSCKASGYTFTENAMHWVR |
| | | QAPGQRLEWIGGINPNQGGTSYSQKFQGRATLTVDTSAST |
| | | AYMELSSLRSEDTAVYYCTRSSYYRYDLDYWGQGTTVTV |
| | | SSASTKGPSVFPLAPSSKSTSGGTAALGCLVKDYFPEPVTV |
| | | SWNSGALTSGVHTFPAVLQSSGLYSLSSVVTVPSSSLGTQT |
| | | YICNVNHKPSNTKVDKRVEPKSCDKTHTCPPCPAPEAEGA |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALPSSIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGK |
| 61 | 6VH6.M7 heavy chain | QVQLVQSGAEVKKPGASVKVSCKASGYTFTENAMHWVR |
| | | QAPGQRLEWIGGINPNNAGTSYSQKFQGRATLTVDTSAST |
| | | AYMELSSLRSEDTAVYYCTRSSYYRYDLDYWGQGTTVTV |
| | | SSASTKGPSVFPLAPSSKSTSGGTAALGCLVKDYFPEPVTV |
| | | SWNSGALTSGVHTFPAVLQSSGLYSLSSVVTVPSSSLGTQT |
| | | YICNVNHKPSNTKVDKRVEPKSCDKTHTCPPCPAPEAEGA |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALPSSIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGK |
| 62 | 6VL1.M1 light chain | DIQMTQSPSSLSASVGDRVTITCQASQDIKTALAWYQQKP |
| | | GKAPKLLIYSASYRYTGVPSRFSGSGSGTDFTFTISSLQPEDI |
| | | ATYYCHQHYTTPWTFGGGTKLEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 63 | TIGIT-19 chimeric | QVQLQQPGPELVKPGTSVMLSCKASGYAFSRYSMYWVKQ |
| | heavy chain | SHGKSLEWIAFIDPYNGGSTYNQRFRGKATLTVDKSSSTAY |
| | | MHLNTLTSDDSAVYYCARGGDYGWYLGVWGAGTTVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQTY |
| | | ICNVNHKPSNTKVDKRVEPKSCDKTHTCPPCPAPEAEGAPS |
| | | VFLFPPKPKDTLMISRTPEVTCVVVDVSHEDPEVKFNWYV |
| | | DGVEVHNAKTKPREEQYNSTYRVVSVLTVLHQDWLNGKE |
| | | YKCKVSNKALPSSIEKTISKAKGQPREPQVYTLPPSREEMT |
| | | KNQVSLTCLVKGFYPSDIAVEWESNGQPENNYKTTPPVLD |
| | | SDGSFFLYSKLTVDKSRWQQGNVFSCSVMHEALHNHYTQ |
| | | KSLSLSPGK |
| 64 | TIGIT-19 chimeric | DIQMTQSPASLSASVGETVTITCRPSENIYTYLSWYQQKRG |
| | light chain | KSPQLLVYNAKTLPENVPSRFSGSGSGTQFSLKIDSLQPEDF |
| | | GNYYCQHHYGNPLTFGAGTKLELKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| 65 | 19VH4.M9 heavy | QVQLVQSGAEVKKPGASVKVSCKASGYTFTRYSMYWVR |
| | chain | QAPGQGLEWIAFIDAYSGGSTYNQRFRGKATLTVDTSISTA |
| | | YMELSRLRSDDTAVYYCARGGDYGWYLGVWGQGTLVTV |
| | | SSASTKGPSVFPLAPSSKSTSGGTAALGCLVKDYFPEPVTV |
| | | SWNSGALTSGVHTFPAVLQSSGLYSLSSVVTVPSSSLGTQT |
| | | YICNVNHKPSNTKVDKRVEPKSCDKTHTCPPCPAPEAEGA |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALPSSIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGK |
| 66 | 19VH4.M10 heavy | QVQLVQSGAEVKKPGASVKVSCKASGYTFTRYSMYWVR |
| | chain | QAPGQGLEWIAFIDAYSGGSTYAQKFQGKATLTVDTSISTA |
| | | YMELSRLRSDDTAVYYCARGGDYGWYLGVWGQGTLVTV |
| | | SSASTKGPSVFPLAPSSKSTSGGTAALGCLVKDYFPEPVTV |
| | | SWNSGALTSGVHTFPAVLQSSGLYSLSSVVTVPSSSLGTQT |
| | | YICNVNHKPSNTKVDKRVEPKSCDKTHTCPPCPAPEAEGA |
| | | PSVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALPSSIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGK |
| 67 | 19VL1.M4 light | DIQMTQSPSSLSASVGDRVTITCRPSENIYTYLSWYQQKPG |
| | chain | KAPKLLIYNAKSLPSGVPSRFSGSGSGTDFTLTISSLQPEDF |
| | | ATYYCQHHYGNPLTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 68 | 19VL1.M7 light | DIQMTQSPSSLSASVGDRVTITCRASQSIYSYLSWYQQKPG |
| | chain | KAPKLLIYNAKSLPSGVPSRFSGSGSGTDFTLTISSLQPEDF |
| | | ATYYCQHHYGNPLTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| 69 | human IgGI heavy | ASTKGPSVFPLAPSSKSTSGGTAALGCLVKDYFPEPVTVSW |
| | chain constant | NSGALTSGVHTFPAVLQSSGLYSLSSVVTVPSSSLGTQTYIC |
| | region | NVNHKPSNTKVDKRVEPKSCDKTHTCPPCPAPEAEGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPSSIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| 70 | human κ chain | RTVAAPSVFIFPPSDEQLKSGTASVVCLLNNFYPREAKVQW |
| | constant region | KVDNALQSGNSQESVTEQDSKDSTYSLSSTLTLSKADYEK |
| | | HKVYACEVTHQGLSSPVTKSFNRGEC |
| 71 | TIGIT-3 | GFTFSSYGMS |
| | HCDR1 | |
| | (3VH/3VH3/3VH3.M4) | |
| 72 | TIGIT-3 | TIKSDGGSTNLPDSVKG |
| | HCDR2-1 | |
| | (3VH/3VH3) | |
| 73 | TIGIT-3 | TIKSSGGSTNLADSVKG |
| | HCDR2-2 | |
| | (3VH3.M4) | |
| 74 | TIGIT-3 | TIKSX₁GGSTNLX₂DSVKG |
| | HCDR2 Formula | |
| 75 | TIGIT-3 | GYFDY |
| | HCDR3 | |
| | (3VH/3VH3/3VH3.M4) | |
| 76 | TIGIT-3 | KASQDVKTAVA |
| | LCDR1-1 | |
| | (3VL/3VL1/3VL1.M3/ | |
| | 3VL1.M4) | |
| 77 | TIGIT-3 | RASQDIKTALA |
| | LCDR1-2 | |
| | (3VL1.M1/3VL4.M6/ | |
| | 3VL5.M6) | |
| 78 | TIGIT-3 | X₃ASQDX₄KTAX₅A |
| | LCDR1 Formula | |
| 79 | TIGIT-3 | WSSTRHT |
| | LCDR2-1 | |
| | (3VL/3VL1/3VL1.M1) | |
| 80 | TIGIT-3 | WASTRHS |
| | LCDR2-2 | |
| | (3VL1.M3) | |
| 81 | TIGIT-3 | WASTRQS |
| | LCDR2-3 | |
| | (3VL1.M4/3VL4.M6/ | |
| | 3VL5.M6) | |
| 82 | TIGIT-3 | WX₆STRX₇X₈ |
| | LCDR2 Formula | |
| 83 | TIGIT-3 | QQYYSTPYT |
| | LCDR3 | |
| | (3VL/3VL1/3VL1.M1/ | |
| | 3VL1.M3/3VL1.M4/ | |
| | 3VL4.M6/3VL5.M6) | |
| 84 | TIGIT-6 | GYTITENTMH |
| | HCDR1-1 | |
| | (6VH/6VH5/6VH6/ | |
| | 6VH6.M3/6VH6.M4/ | |
| | 6VH6.M5) | |
| 85 | TIGIT-6 | GYTFTENAMH |
| | HCDR1-2 | |
| | (6VH6.M1/6VH6.M6/ | |
| | 6VH6.M7) | |
| 86 | TIGIT-6 | GYTX₁TENX₂MH |
| | HCDR1 Formula | |
| 87 | TIGIT-6 | GINPNNGGTSYNQQFKG |
| | HCDR2-1 | |
| | (6VH/6VH5/6VH6/ | |
| | 6VH6.M1) | |
| 88 | TIGIT-6 | GINPNNGGTSYSQKFQG |
| | HCDR2-2 | |
| | (6VH6.M3) | |
| 89 | TIGIT-6 | GINPNQGGTSYSQKFQG |
| | HCDR2-3 | |
| | (6VH6.M4/6VH6.M6) | |
| 90 | TIGIT-6 | GINPNNAGTSYSQKFQG |
| | HCDR2-4 | |
| | (6VH6.M5/6VH6.M7) | |
| 91 | TIGIT-6 | GINPNX₃X₄GTSYX₅QX₆FX₇G |
| | HCDR2 Formula | |
| 92 | TIGIT-6 | SSYYRYDLDY |
| | HCDR3 | |
| | (6VH/6VH5/6VH6/ | |
| | 6VH6.M1/6VH6.M3/ | |
| | 6VH6.M4/6VH6.M5/ | |
| | 6VH6.M6/6VH6.M7) | |
| 93 | TIGIT-6 | KASQDVKTALA |
| | LCDR1-1 | |
| | (6VL/6VL1) | |
| 94 | TIGIT-6 | QASQDIKTALA |
| | LCDR1-2 | |
| | (6VL1.M1) | |
| 95 | TIGIT-6 | X₈ASQDX₉KTALA |
| | LCDR1 Formula | |
| 96 | TIGIT-6 | SASYRYT |
| | LCDR2 | |
| | (6VL/6VL1/6VL1.M1) | |
| 97 | TIGIT-6 | HQHYTTPWT |
| | LCDR3 | |
| | (6VL/6VL1/6VL1.M1) | |
| 98 | TIGIT-19 | GYAFSRYSMY |
| | HCDR1-1 | |
| | (19VH/19VH4/ | |
| | 19VH4.M4/19VH4.M5) | |
| 99 | TIGIT-19 | GYTFTRYSMY |
| | HCDR1-2 | |
| | (19VH4.M6/19VH4.M7/ | |
| | 19VH4.M9/19VH4.M10) | |
| 100 | TIGIT-19 | GYX₁FX₂RYSMY |
| | HCDR1 Formula | |
| 101 | TIGIT-19 | FIDPYNGGSTYNQRFRG |
| | HCDR2-1 | |
| | (19VH/19VH4) | |
| 102 | TIGIT-19 | FIDPYSGGSTYNQRFRG |
| | HCDR2-2 | |
| | (19VH4.M4/19VH4.M6) | |
| 103 | TIGIT-19 | FIDPYNGGSTYAQKFQG |
| | HCDR2-3 | |
| | (19VH4.M5) | |
| 104 | TIGIT-19 | FIDPYSGGSTYAQKFQG |
| | HCDR2-4 | |
| | (19VH4.M7) | |
| 105 | TIGIT-19 | FIDAYSGGSTYNQRFRG |
| | HCDR2-5 | |
| | (19VH4.M9) | |
| 106 | TIGIT-19 | FIDAYSGGSTYAQKFQG |
| | HCDR2-6 | |
| | (19VH4.M10) | |
| 107 | TIGIT-19 | FIDX₃YX₄GGSTYX₅QX₆FX₇G |
| | HCDR2 Formula | |
| 108 | TIGIT-19 | GGDYGWYLGV |
| | HCDR3 | |
| | (19VH/19VH4/ | |
| | 19VH4.M4/19VH4.M5/ | |
| | 19VH4.M6/19VH4.M7/ | |
| | 19VH4.M9/19VH4.M10) | |
| 109 | TIGIT-19 | RPSENIYTYLS |
| | LCDR1-1 | |
| | (19VL/19VL1/ | |
| | 19VL1.M4) | |
| 110 | TIGIT-19 | RASQNIYTYLS |
| | LCDR1-2 | |
| | (19VL1.M5) | |
| 111 | TIGIT-19 | RASQNIYSYLS |
| | LCDR1-3 | |
| | (19VL1.M6) | |
| 112 | TIGIT-19 | RASQSIYSYLS |
| | LCDR1-4 | |
| | (19VL1.M7) | |
| 113 | TIGIT-19 | RX₈SX₉X₁₀IYX₁₁YLS |
| | LCDR1 Formula | |
| 114 | TIGIT-19 | NAKTLPE |
| | LCDR2-1 | |
| | (19VL/19VL1) | |
| 115 | TIGIT-19 | NAKSLPS |
| | LCDR2-2 | |
| | (19VL1.M4/19VL1.M5/ | |
| | 19VL1.M6/19VL1.M7) | |
| 116 | TIGIT-19 | NAKX₁₂LPX₁₃ |
| | LCDR2 Formula | |
| 117 | TIGIT-19 | QHHYGNPLT |
| | LCDR3 | |
| | (19VL/19VL1/ | |
| | 19VL1.M4/19VL1.M5/ | |
| | 19VL1.M6/19VL1.M7) | |
| 118 | TIGIT-15 | GFTFSSYGMS |
| | HCDR1 | |
| 119 | TIGIT-15 | TINNNGGSTYYPDSVKG |
| | HCDR2 | |
| 120 | TIGIT-15 | RTGTEILGFDF |
| | HCDR3 | |
| 121 | TIGIT-15 | KASQDVSTAVA |
| | LCDR1 | |
| 122 | TIGIT-15 | WASSRHT |
| | LCDR2 | |
| 123 | TIGIT-15 | QQQYSTPWT |
| | LCDR3 | |
| 124 | TIGIT-18 | GYSFTSYWMN |
| | HCDR1 | |
| 125 | TIGIT-18 | MIHLSNSETRLNQNFLD |
| | HCDR2 | |
| 126 | TIGIT-18 | ERDYYGSSYFDY |
| | HCDR3 | |
| 127 | TIGIT-18 | RTSQSVSTSSYSYMH |
| | LCDR1 | |
| 128 | TIGIT-18 | NASNLES |
| | LCDR2 | |
| 129 | TIGIT-18 | QHSWEIPRT |
| | LCDR3 | |
| 130 | TIGIT-52 | GFTFSSFAMS |
| | HCDR1 | |
| 131 | TIGIT-52 | TISNGGSYTYYPDTVKG |
| | HCDR2 | |
| 132 | TIGIT-52 | PYYGTTYGYYFDF |
| | HCDR3 | |
| 133 | TIGIT-52 | RASESVDRNGNSFMH |
| | LCDR1 | |
| 134 | TIGIT-52 | LASNLES |
| | LCDR2 | |
| 135 | TIGIT-52 | QQNNEDPLT |
| | LCDR3 | |
| 136 | TIGIT-63 | GFTFTDYYMN |
| | HCDR1 | |
| 137 | TIGIT-63 | FIRNKANGYTTEYSASVKG |
| | HCDR2 | |
| 138 | TIGIT-63 | STAWYFDV |
| | HCDR3 | |
| 139 | TIGIT-63 | RASEHIYSYLA |
| | LCDR1 | |
| 140 | TIGIT-63 | NAKTLAE |
| | LCDR2 | |
| 141 | TIGIT-63 | QHHYGNPLT |
| | LCDR3 | |
| 142 | TIGIT-87 | GYSFTGYTMN |
| | HCDR1 | |
| 143 | TIGIT-87 | LINPYNGGTSYNQKFKG |
| | HCDR2 | |
| 144 | TIGIT-87 | YANRFENWFAY |
| | HCDR3 | |
| 145 | TIGIT-87 | RASENIYSYLA |
| | LCDR1 | |
| 146 | TIGIT-87 | NAKTLAE |
| | LCDR2 | |
| 147 | TIGIT-87 | QHHYGTPWT |
| | LCDR3 | |
| 148 | TIGIT-94 | GFTFSTYTMS |
| | HCDR1 | |
| 149 | TIGIT-94 | TISSGGTYTHYPDSVKG |
| | HCDR2 | |
| 150 | TIGIT-94 | WFLSLYAMDY |
| | HCDR3 | |
| 151 | TIGIT-94 | RASQDINNFLN |
| | LCDR1 | |
| 152 | TIGIT-94 | YTSRLHS |
| | LCDR2 | |
| 153 | TIGIT-94 | QQGNTFPYT |
| | LCDR3 | |
| 154 | TIGIT-100 | GYSFTDYNMN |
| | HCDR1 | |
| 155 | TIGIT-100 | VINPNSGTDNYNQKFKG |
| | HCDR2 | |
| 156 | TIGIT-100 | GVYFGNYFDS |
| | HCDR3 | |
| 157 | TIGIT-100 | RASENIYSSLA |
| | LCDR1 | |
| 158 | TIGIT-100 | NAKTLTE |
| | LCDR2 | |
| 159 | TIGIT-100 | QHHYGAPWT |
| | LCDR3 | |

1. An antibody or antigen-binding fragment thereof, that binds to TIGIT, comprising:
(i) HCDR1 comprising the sequence of SEQ ID NO: 84-86, 98-100, 71, 118, 124, 130, 136, 142, 148, or 154;
(ii) HCDR2 comprising the sequence of SEQ ID NO: 87-91, 101-107, 72-74, 119, 125, 131, 137, 143, 149, or 155;
(iii) HCDR3 comprising the sequence of SEQ ID NO: 92, 108, 75, 120, 126, 132, 138, 144, 150, or 156;
(iv) LCDR1 comprising the sequence of SEQ ID NO: 93-95, 109-113, 76-78, 121, 127, 133, 139, 145, 151, or 157;
(v) LCDR2 comprising the sequence of SEQ ID NO: 96, 114-116, 79-82, 122, 128, 134, 140, 146, 152, or 158; and
(vi) LCDR3 comprising the sequence of SEQ ID NO: 97, 117, 83, 123, 129, 135, 141, 147, 153, or 159.

2. The antibody or antigen-binding fragment thereof of claim 1, comprising:
(a) (i) HCDR1 comprising the sequence of GYTX₁TENX₂MH (SEQ ID NO: 86), wherein X₁ is I or F, X₂ is T or A;
(ii) HCDR2 comprising the sequence of GINPNX₃X₄GTSYX₅QX₆FX₇G (SEQ ID NO: 91), wherein X₃ is N or Q, X₄ is G or A, X₅ is N or S, X₆ is Q or K, X₇ is K or Q;
(iii) HCDR3 comprising the sequence of SEQ ID NO: 92;
(iv) LCDR1 comprising the sequence of X₈ASQDX₉KTALA (SEQ ID NO: 95), wherein X₈ is K or Q, X₉ is V or I;
(v) LCDR2 comprising the sequence of SEQ ID NO: 96; and
(vi) LCDR3 comprising the sequence of SEQ ID NO: 97; or
(b) (i) HCDR1 comprising the sequence of GYX₁FX₂RYSMY (SEQ ID NO: 100), wherein X₁ is A or T, X₂ is S or T;
(ii) HCDR2 comprising the sequence of FIDX₃YX₄GGSTYX₅QX₆FX-G (SEQ ID NO: 107), wherein X₃ is P or A, X₄ is N or S, X₅ is N or A, X₆ is R or K, X₇ is R or Q;
(iii) HCDR3 comprising the sequence of SEQ ID NO: 108;
(iv) LCDR1 comprising the sequence of RX₈SX₉X₁₀IYX₁₁YLS (SEQ ID NO: 113), wherein X₈ is P or A, X₉ is E or Q, X₁₀ is N or S, X₁₁ is T or S;
(v) LCDR2 comprising the sequence of NAKX₁₂LPX₁₃ (SEQ ID NO: 116), wherein X₁₂ is T or S, X₁₃ is E or S; and
(vi) LCDR3 comprising the sequence of SEQ ID NO: 117; or
(c) (i) HCDR1 comprising the sequence of SEQ ID NO: 71;
(ii) HCDR2 comprising the sequence of TIKSX₁GGSTNLX₂DSVKG (SEQ ID NO: 74), wherein X₁ is D or S, X₂ is P or A;
(iii) HCDR3 comprising the sequence of SEQ ID NO: 75;
(iv) LCDR1 comprising the sequence of X₃ASQDX₄KTAX₅A (SEQ ID NO: 78), wherein X₃ is K or R, X₄ is V or I, X₅ is V or L;
(v) LCDR2 comprising the sequence of WX₆STRX₇X₈ (SEQ ID NO: 82), wherein X₆ is S or A, X₇ is H or Q, X₈ is T or S; and
(vi) LCDR3 comprising the sequence of SEQ ID NO: 83.

3. The antibody or antigen-binding fragment thereof of claim 1, comprising:
(a1) HCDR1, HCDR2 and HCDR3 comprising the amino acid sequences of SEQ ID NOs: 84, 87, and 92, respectively; and LCDR1, LCDR2 and LCDR3 comprising the amino acid sequences of SEQ ID NOs: 93, 96, and 97, respectively;
(a2) HCDR1, HCDR2 and HCDR3 comprising the amino acid sequences of: (a) SEQ ID NOs: 85, 87, and 92, respectively; or (b) SEQ ID NOs: 84, 88, and 92, respectively; or (c) SEQ ID NOs: 84, 89, and 92, respectively; and LCDR1, LCDR2 and LCDR3 comprising the amino acid sequences of SEQ ID NOs: 93, 96, and 97, respectively; or,
(a3) HCDR1, HCDR2 and HCDR3 comprising the amino acid sequences of: (a) SEQ ID NOs: 84, 87, and 92, respectively; or (b) SEQ ID NOs: 84, 88, and 92, respectively; or (c) SEQ ID NOs: 84, 89, and 92, respectively; or (d) SEQ ID NOs: 84, 90, and 92, respectively; or (e) SEQ ID NOs: 85, 89, and 92, respectively; or (f) SEQ ID NOs: 85, 90, and 92, respectively; and LCDR1, LCDR2 and LCDR3 comprising the amino acid sequences of SEQ ID NOs: 94, 96, and 97, respectively;
(b1) HCDR1, HCDR2 and HCDR3 comprising the amino acid sequences of SEQ ID NOs: 98, 101, and 108, respectively; and LCDR1, LCDR2 and LCDR3 comprising the amino acid sequences of SEQ ID NOs: 109, 114, and 117, respectively;
(b2) HCDR1, HCDR2 and HCDR3 comprising the amino acid sequences of SEQ ID NOS: 98, 101 and 108, respectively; and LCDR1, LCDR2 and LCDR3 comprising the amino acid sequences of: (a) SEQ ID NOs: 109, 115 and 117, respectively; or (b) SEQ ID NOs: 110, 115 and 117, respectively; or (c) SEQ ID NOs: 111, 115 and 117, respectively; or (d) SEQ ID NOS: 112, 115 and 117, respectively;
(b3) HCDR1, HCDR2 and HCDR3 comprising the amino acid sequences of: (a) SEQ ID NOs: 98, 102, and 108, respectively; or (b) SEQ ID NOs: 98, 103, and 108, respectively; or (c) SEQ ID NOs: 99, 102, and 108, respectively; or (d) SEQ ID NOs: 99, 104, and 108, respectively; or (e) SEQ ID NOs: 99, 105, and 108, respectively; and LCDR1, LCDR2 and LCDR3 comprising the amino acid sequences of SEQ ID NOs: 109, 114 and 117, respectively;
(b4) HCDR1, HCDR2 and HCDR3 comprising the amino acid sequences of SEQ ID NOs: 99, 105 and 108, respectively; and LCDR1, LCDR2 and LCDR3 comprising the amino acid sequences of: (a) SEQ ID NOs: 109, 115 and 117, respectively; or (b) SEQ ID NOs: 112, 115 and 117, respectively;
(b5) HCDR1, HCDR2 and HCDR3 comprising the amino acid sequences of SEQ ID NOs: 99, 106 and 108, respectively; and LCDR1, LCDR2 and LCDR3 comprising the amino acid sequences of: (a) SEQ ID NOs: 109, 115 and 117, respectively; or (b) SEQ ID NOs: 112, 115 and 117, respectively;
(c1) HCDR1, HCDR2 and HCDR3 comprising the amino acid sequences of SEQ ID NOs: 71, 72, and 75, respectively; and LCDR1, LCDR2 and LCDR3 comprising the amino acid sequences of SEQ ID NOs: 76, 79, and 83, respectively;
(c2) HCDR1, HCDR2 and HCDR3 comprising the amino acid sequences of SEQ ID NOs: 71, 73, and 75, respectively; and LCDR1, LCDR2 and LCDR3 comprising the amino acid sequences of SEQ ID NOs: 76, 79, and 83, respectively;
(c3) HCDR1, HCDR2 and HCDR3 comprising the amino acid sequences of SEQ ID NOs: 71, 72, and 75; and LCDR1, LCDR2 and LCDR3 comprising the amino acid sequences of: (a) SEQ ID NOs: 76, 79, and 83, respectively; or (b) SEQ ID NOs: 77, 79, and 83, respectively; or
(c) SEQ ID NOs: 76, 80, and 83, respectively; or (d) SEQ ID NOs: 76, 81, and 83, respectively; or
(c4) HCDR1, HCDR2 and HCDR3 comprising the amino acid sequences of SEQ ID NOs: 71, 73, and 75, respectively; and LCDR1, LCDR2 and LCDR3 comprising the amino acid sequences of SEQ ID NOs: 77, 81, and 83, respectively.

4. The antibody or antigen-binding fragment thereof of claim 1, comprising a heavy chain variable region (VH) and a light chain variable region (VL), wherein:
(1) the VH comprises the sequence of SEQ ID NO: 4 or an amino acid sequence having at least 80% sequence identity thereto; and/or, the VL comprises the sequence of SEQ ID NO: 5 or an amino acid sequence having at least 80% sequence identity thereto;
(2) the VH comprises the sequence of SEQ ID NO: 10 or an amino acid sequence having at least 80% sequence identity thereto; and/or, the VL comprises the sequence of SEQ ID NO: 11 or an amino acid sequence having at least 80% sequence identity thereto; or
(3) the VH comprises the sequence of SEQ ID NO: 2 or an amino acid sequence having at least 80% sequence identity thereto; and/or, the VL comprises the sequence of SEQ ID NO: 3 or an amino acid sequence having at least 80% sequence identity thereto.

5. The antibody or antigen-binding fragment thereof of claim 1, comprising a heavy chain variable region (VH) and a light chain variable region (VL), wherein:
(1) the VH comprises framework regions (FRs) derived from a human immunoglobulin heavy chain variable region sequence of GenBank: QVG74320; and/or, the VL comprises framework regions (FRs) derived from a human immunoglobulin light chain variable region sequence of GenBank: ACY78416;
(2) the VH comprises framework regions (FRs) derived from a human immunoglobulin heavy chain variable region sequence of GenBank: AXA20212; and/or, the VL comprises framework regions (FRs) derived from a human immunoglobulin light chain variable region sequence of GenBank: ABA70776; or
(3) the VH comprises framework regions (FRs) derived from a human immunoglobulin heavy chain variable region sequence of GenBank: BAC02278; and/or, the VL comprises framework regions (FRs) derived from a human immunoglobulin light chain variable region sequence of GenBank: QTX15665.

6. The antibody or antigen-binding fragment thereof of claim 5, wherein:
(a1) the VH comprises the sequence of SEQ ID NO: 30, 31, 33, 34, or 36, or an amino acid sequence having at least 80% sequence identity thereto; and/or, the VL comprises the sequence of SEQ ID NO: 32 or an amino acid sequence having at least 80% sequence identity thereto;
(a2) the VH comprises the sequence of SEQ ID NO: 31, 34, 36, 37, 38, or 39, or an amino acid sequence having at least 80% sequence identity thereto; and/or, the VL comprises the sequence of SEQ ID NO: 35 or an amino acid sequence having at least 80% sequence identity thereto;
(b1) the VH comprises the sequence of SEQ ID NO: 40, or an amino acid sequence having at least 80% sequence identity thereto; and/or, the VL comprises the sequence of SEQ ID NO: 41, 42, 43, 44, or 45, or an amino acid sequence having at least 80% sequence identity thereto;
(b2) the VH comprises the sequence of SEQ ID NO: 46, 47, 48, 49, or 50, or an amino acid sequence having at least 80% sequence identity thereto; and/or, the VL comprises the sequence of SEQ ID NO: 41 or an amino acid sequence having at least 80% sequence identity thereto;
(b3) the VH comprises the sequence of SEQ ID NO: 50, or an amino acid sequence having at least 80% sequence identity thereto; and/or, the VL comprises the sequence of SEQ ID NO: 42 or 45, or an amino acid sequence having at least 80% sequence identity thereto;
(b4) the VH comprises the sequence of SEQ ID NO: 51, or an amino acid sequence having at least 80% sequence identity thereto; and/or, the VL comprises the sequence of SEQ ID NO: 42 or 45, or an amino acid sequence having at least 80% sequence identity thereto;
(c1) the VH comprises the sequence of SEQ ID NO: 22 or 23, or an amino acid sequence having at least 80% sequence identity thereto; and/or, the VL comprises the sequence of SEQ ID NO: 3 or an amino acid sequence having at least 80% sequence identity thereto;
(c2) the VH comprises the sequence of SEQ ID NO: 22, or an amino acid sequence having at least 80% sequence identity thereto; and/or, the VL comprises the sequence of SEQ ID NO: 24, 25, 26, or 27, or an amino acid sequence having at least 80% sequence identity thereto; or
(c3) the VH comprises the sequence of SEQ ID NO: 23, or an amino acid sequence having at least 80% sequence identity thereto; and/or, the VL comprises the sequence of SEQ ID NO: 28 or 29, or an amino acid sequence having at least 80% sequence identity thereto.

7.-16. (canceled)

17. The antibody or antigen-binding fragment thereof of claim 1, comprising:
(1) HCDR1, HCDR2, HCDR3, LCDR1, LCDR2 and LCDR3 comprising the amino acid sequences of SEQ ID NOs: 118, 119, 120, 121, 122, and 123, respectively;
(2) HCDR1, HCDR2, HCDR3, LCDR1, LCDR2 and LCDR3 comprising the amino acid sequences of SEQ ID NOs: 124, 125, 126, 127, 128, and 129, respectively;
(3) HCDR1, HCDR2, HCDR3, LCDR1, LCDR2 and LCDR3 comprising the amino acid sequences of SEQ ID NOs: 130, 131, 132, 133, 134, and 135, respectively;
(4) HCDR1, HCDR2, HCDR3, LCDR1, LCDR2 and LCDR3 comprising the amino acid sequences of SEQ ID NOs: 136, 137, 138, 139, 140, and 141, respectively;
(5) HCDR1, HCDR2, HCDR3, LCDR1, LCDR2 and LCDR3 comprising the amino acid sequences of SEQ ID NOs: 142, 143, 144, 145, 146 and 147, respectively;
(6) HCDR1, HCDR2, HCDR3, LCDR1, LCDR2 and LCDR3 comprising the amino acid sequences of SEQ ID NOs: 148, 149, 150, 151, 152 and 153, respectively; or
(7) HCDR1, HCDR2, HCDR3, LCDR1, LCDR2 and LCDR3 comprising the amino acid sequences of SEQ ID NOs: 154, 155, 156, 157, 158 and 159, respectively.

18. The antibody or antigen-binding fragment thereof of claim 1, further comprising: a heavy chain constant region (CH) comprising an amino acid sequence derived from a human immunoglobulin heavy chain constant region, wherein
the heavy chain constant region is an IgG heavy chain constant region, such as an IgG1, IgG2, IgG3 or IgG4 heavy chain constant region;
and/or a light chain constant region (C_{L}) comprising an amino acid sequence derived from a human immunoglobulin light chain constant region, wherein
the light chain constant region is a kappa light chain constant region; preferably, the light chain constant region comprises the amino acid sequence of SEQ ID NO: 70.

19. The antibody or antigen-binding fragment thereof of claim 18, comprising:
(1) a heavy chain comprising the sequence of SEQ ID NO: 56, 58, 59, 60, or 61, or an amino acid sequence having at least 80% sequence identity thereto; and/or,
a light chain comprising the sequence of SEQ ID NO: 57 or 62, or an amino acid sequence having at least 80% sequence identity thereto;
(2) a heavy chain comprising the sequence of SEQ ID NO: 63, 65, or 66, or an amino acid sequence having at least 80% sequence identity thereto; and/or,
a light chain comprising the sequence of SEQ ID NO: 64, 67, or 68, or an amino acid sequence having at least 80% sequence identity thereto; or
(3) a heavy chain comprising the sequence of SEQ ID NO: 52 or 54, or an amino acid sequence having at least 80% sequence identity thereto; and/or,
a light chain comprising the sequence of SEQ ID NO: 53 or 55, or an amino acid sequence having at least 80% sequence identity thereto.

20. (canceled)

21. (canceled)

22. The antibody or antigen-binding fragment thereof of claim 1, wherein the antibody or antigen-binding fragment thereof is selected from the group consisting of scFv, Fab, Fab′, (Fab′)₂, a Fv fragment, a diabody, a bispecific antibody, a multispecific antibody, a chimeric antibody, and a humanized antibody.

23.-29. (canceled)

30. A pharmaceutical composition, comprising the antibody or antigen-binding fragment thereof of claim 1, and a pharmaceutically acceptable carrier and/or excipient.

31. The pharmaceutical composition of claim 30, which further comprises an additional therapeutic agent.

32. (canceled)

33. (canceled)

34. A method of (a) increasing immune cell activation in response to an antigen in a subject; (b) enhancing an immune response in a subject; (c) reducing or eliminating TIGIT-mediated immune suppression in a subject; (d) reducing or depleting regulatory T cells in a tumor of a subject; (e) treating tumor in a subject; and/or (f) treating an infectious disease in a subject; wherein the method comprising administering to a subject in need thereof a therapeutically effective amount of the antibody or antigen-binding fragment thereof of claim 1

35. The antibody or antigen-binding fragment thereof of claim 5, comprising a heavy chain variable region (VH) and a light chain variable region (VL), wherein
(1) the VH comprises the sequence of SEQ ID NO: 30, 31, 33, 34, 36, 37, 38, or 39, or an amino acid sequence having at least 80% sequence identity thereto; and/or, the VL comprises the sequence of SEQ ID NO: 32 or 35, or an amino acid sequence having at least 80% sequence identity thereto;
(2) the VH comprises the sequence of SEQ ID NO: 40, 46, 47, 48, 49, 50, or 51, or an amino acid sequence having at least 80% sequence identity thereto; and/or, the VL comprises the sequence of SEQ ID NO: 41, 42, 43, 44, or 45, or an amino acid sequence having at least 80% sequence identity thereto; or
(3) the VH comprises the sequence of SEQ ID NO: 22 or 23, or an amino acid sequence having at least 80% sequence identity thereto; and/or, the VL comprises the sequence of SEQ ID NO: 24, 25, 26, 27, 28, or 29, or an amino acid sequence having at least 80% sequence identity thereto.

36. The antibody or antigen-binding fragment thereof of claim 5, wherein
(a1) the VH comprises the sequence of SEQ ID NO: 36; and/or, the VL comprises the sequence of SEQ ID NO: 35;
(a2) the VH comprises the sequence of SEQ ID NO: 37; and/or, the VL comprises the sequence of SEQ ID NO: 35;
(a3) the VH comprises the sequence of SEQ ID NO: 38; and/or, the VL comprises the sequence of SEQ ID NO: 35;
(a4) the VH comprises the sequence of SEQ ID NO: 39; and/or, the VL comprises the sequence of SEQ ID NO: 35;
(b1) the VH comprises the sequence of SEQ ID NO: 50; and/or, the VL comprises the sequence of SEQ ID NO: 42;
(b2) the VH comprises the sequence of SEQ ID NO: 50; and/or, the VL comprises the sequence of SEQ ID NO: 45;
(b3) the VH comprises the sequence of SEQ ID NO: 51; and/or, the VL comprises the sequence of SEQ ID NO: 42;
(b4) the VH comprises the sequence of SEQ ID NO: 51; and/or, the VL comprises the sequence of SEQ ID NO: 45; or
(c) the VH comprises the sequence of SEQ ID NO: 23; and/or, the VL comprises the sequence of SEQ ID NO: 28.

37. The antibody or antigen-binding fragment thereof of claim 1, comprising:
(1) a VH comprising the sequence of SEQ ID NO: 6, or an amino acid sequence having at least 80% sequence identity thereto; and/or, a VL comprising the sequence of SEQ ID NO: 7, or an amino acid sequence having at least 80% sequence identity thereto;
(2) a VH comprising the sequence of SEQ ID NO: 8, or an amino acid sequence having at least 80% sequence identity thereto; and/or, a VL comprising the sequence of SEQ ID NO: 9, or an amino acid sequence having at least 80% sequence identity thereto;
(3) a VH comprising the sequence of SEQ ID NO: 12, or an amino acid sequence having at least 80% sequence identity thereto; and/or, a VL comprising the sequence of SEQ ID NO: 13, or an amino acid sequence having at least 80% sequence identity thereto;
(4) a VH comprising the sequence of SEQ ID NO: 14, or an amino acid sequence having at least 80% sequence identity thereto; and/or, a VL comprising the sequence of SEQ ID NO: 15, or an amino acid sequence having at least 80% sequence identity thereto;
(5) a VH comprising the sequence of SEQ ID NO: 16, or an amino acid sequence having at least 80% sequence identity thereto; and/or, a VL comprising the sequence of SEQ ID NO: 17, or an amino acid sequence having at least 80% sequence identity thereto;
(6) a VH comprising the sequence of SEQ ID NO: 18, or an amino acid sequence having at least 80% sequence identity thereto; and/or, a VL comprising the sequence of SEQ ID NO: 19, or an amino acid sequence having at least 80% sequence identity thereto; or,
(7) a VH comprising the sequence of SEQ ID NO: 20, or an amino acid sequence having at least 80% sequence identity thereto; and/or, a VL comprising the sequence of SEQ ID NO: 21, or an amino acid sequence having at least 80% sequence identity thereto.

38. The antibody or antigen-binding fragment thereof of claim 18, wherein the heavy chain constant region comprises the amino acid sequence of SEQ ID NO: 69; and/or the light chain constant region comprises the amino acid sequence of SEQ ID NO: 70.

39. The antibody or antigen-binding fragment thereof of claim 18, comprising:
(a1) a heavy chain comprising the sequence of SEQ ID NO: 56, or an amino acid sequence having at least 80% sequence identity thereto; and/or, a light chain comprising the sequence of SEQ ID NO: 57, or an amino acid sequence having at least 80% sequence identity thereto;
(a2) a heavy chain comprising the sequence of SEQ ID NO: 58, 59, 60, or 61, or an amino acid sequence having at least 80% sequence identity thereto; and/or, a light chain comprising the sequence of SEQ ID NO: 62, or an amino acid sequence having at least 80% sequence identity thereto;
(b1) a heavy chain comprising the sequence of SEQ ID NO: 63, or an amino acid sequence having at least 80% sequence identity thereto; and/or, a light chain comprising the sequence of SEQ ID NO: 64, or an amino acid sequence having at least 80% sequence identity thereto;
(b2) a heavy chain comprising the sequence of SEQ ID NO: 65, or an amino acid sequence having at least 80% sequence identity thereto; and/or, a light chain comprising the sequence of SEQ ID NO: 67 or 68, or an amino acid sequence having at least 80% sequence identity thereto;
(b3) a heavy chain comprising the sequence of SEQ ID NO: 66, or an amino acid sequence having at least 80% sequence identity thereto; and/or, a light chain comprising the sequence of SEQ ID NO: 67 or 68, or an amino acid sequence having at least 80% sequence identity thereto;
(c1) a heavy chain comprising the sequence of SEQ ID NO: 52, or an amino acid sequence having at least 80% sequence identity thereto; and/or, a light chain comprising the sequence of SEQ ID NO: 53, or an amino acid sequence having at least 80% sequence identity thereto; or,
(c2) a heavy chain comprising the sequence of SEQ ID NO: 54, or an amino acid sequence having at least 80% sequence identity thereto; and/or, a light chain comprising the sequence of SEQ ID NO: 55, or an amino acid sequence having at least 80% sequence identity thereto.

40. The pharmaceutical composition of claim 31, wherein the additional therapeutic agent is an anti-tumor agent, an additional immune checkpoint inhibitor, or a cytotoxic agent.

41. The method of claim 34, wherein the antibody or antigen-binding fragment thereof is used in combination with an additional therapeutic agent or an additional therapy.
